(19) United States
(12) Patent Application Publication
(10) Pub. No.: US 2008/0292591 A1
(43) Pub. Date: Nov. 27, 2008

(54) Tumor mouse models using lentiviral vectors

(75) Inventors: Tomotoshi Marumoto, La Jolla, CA (US); Inder Verma, La Jolla, CA (US); Yasushi Soda, La Jolla, CA (US); Yifeng Xia, San Diego, CA (US)

Correspondence Address: QUINE INTELLECTUAL PROPERTY LAW GROUP, P.C., P O BOX 458, ALAMEDA, CA, 94501, US

(73) Assignee: The Salk Institute for Biological Studies, La Jolla, CA (US)

(21) Appl. No.: 12/074,854

(22) Filed: Mar. 5, 2008

Related U.S. Application Data
(60) Provisional application No. 60/905,226, filed on Mar. 5, 2007.

(51) Int. Cl. A61K 31/7088; A61K 35/12; A01K 67/027; A61P 35/00; G01N 33/00

(52) U.S. Cl. 424/93.1; 514/44; 800/13; 800/18; 800/14; 800/3

(57) ABSTRACT

This invention provides model systems for study of tumors. Model cells and model animals are prepared to mimic characteristics of natural clinical tumors. The tumor models can be contacted with putative modulators to screen for therapeutics and to help understand tumorigenic processes. Model animals can include model cells, closely correlated to the characteristics of clinical tumors, in an environment closely mimicking that of the tumors in patients. Thus, the animal models are highly representative for use in clinical and pharmacological studies.

### CROSS-REFERENCE TO RELATED APPLICATIONS

This application claims priority to and benefit of prior U.S. Provisional Application No. 60/905,226, New Tumor Mouse Models Using CRE Inducible Lentiviral Vectors, by Tomotoshi Marumoto, et al., filed Mar. 5, 2007. The full disclosure of this prior application is incorporated herein by reference.

### FIELD OF THE INVENTION

The present invention is in the field of methods, compositions and systems for modeling tumorigenesis and tumors in model cells and model animals. Lentiviral packaging systems are used to transduce stem cells or differentiated cells at isolated foci in animal organs to generate tumor cells more representative of tumors afflicting adult animals.

### BACKGROUND OF THE INVENTION

Current tumor model technology typically employs a shotgun approach that generates random or unrepresentative tumors. In many cases cells in the tissue or organ are manipulated at suppressor or onco-genes and undergo tumorigenesis en mass. This is a poor model for in vivo tumorigenesis, because tumors more typically initiate by activating oncogenes or inactivating suppressor genes in perhaps a single cell, and then progress to a malignant phenotype by accumulation of additional mutations in a stochastic manner. Furthermore, the current technology does not take into account the role and milieu of the normal cells surrounding a mutated cell.

For example, presently available rodent tumor models rely on four general approaches:

1) External/dietary ingestion of carcinogens, with or without induction of oncogenes.

2) Xenografts, i.e. taking human tumor biopsies and transplanting them in nude mice.

3) Transgenic mice.

4) Activation of oncogenes using retroviral vectors.

Most of these model systems have been used for both drug testing and for studying molecular mechanisms of oncogensis. Unfortunately, most mouse tumor models do not faithfully mimic human tumors in adults. In order to recapitulate what actually happens in tumorigenesis in adults, it is necessary to induce oncogenic events in adult mice in a cell type and microenvironment specific manner. However, all four models shown above fail to provide these aspects.

Models using carcinogens can cause tumors in adult mice with similar histological character to human tumors. However, the carcinogens randomly cause the mutations in any genes, so the tumorigenesis is not reproducible and one cannot know the primary cause of the tumor.

Xenograft models are highly reproducible in adult mice, however the pathological findings of the tumor are often different from the original tumor. For example, glioblastoma, which is the most aggressive brain tumor, has a strongly infiltrative character, while the xenograft models of the glioblastoma do not display this property.

The transgenic/knock out models of tumorigenesis rely on either activation of oncogenes or inactivation of suppressor genes. However, these models often result in tumors that uncontrollably vary in type, location, timing and dispersion.

Activation of oncogenes using common retroviral vectors can establish somatic mutations in adult mice. However, many of these vectors can only infect dividing cells, thus they are often injected into neonatal mice and not into adult mice. Again, the resultant tumors can be unpredictable and unrepresentative.

Modeling for Glioblastoma multiforme (GBM), i.e., gliomas, has been particularly problematic. Gliomas are the most common tumors of the central nervous system in adults, whereas medulloblastomas, a form of primitive neuroectodermal tumor (PNET), is the most frequent central nervous tumor in children [6]. The most widely used current classification of human gliomas is that of the World Health Organization which divides diffuse gliomas into astrocytic tumors, oligodendrogliomas, and oligoastrocytomas. These are further graded into histological degrees of malignancy. Oligodendrogliomas and oligoastrocytomas are tiered into grade II and anaplastic, grade III lesions. The astrocytomas include grade II, grade III, and grade IV lesions, with grade IV known as glioblastoma multiforme (GBM). GBMs are the most common and the most aggressive of the central nervous tumors. They show infiltrative (diffuse) growth character, which essentially prevents surgical resection, and the majority of these GBMs are resistant to standard chemotherapeutic and radiotherapeutic approaches. Due to the resistance of GBMs to several therapeutic approaches, most of the patients with GBMs die of their disease within a median of a year from the time of diagnosis and no patients have long-term survival [7]. Therefore, there is an urgent need to develop accurate models for this disease, to facilitate development of novel therapeutic strategies.

Gliomas often produce various growth factors, such as epidermal growth factor (EGF), platelet-derived growth factor (PDGF) A and B, transforming growth factor-α (TGF-α), and insulin-like growth factor-I (IGF-I). The corresponding receptors, EGFR, PDGF-α and -β, and IGFR, are also frequently over-expressed, sometimes due to amplification, making autocrine or paracrine receptor stimulation likely [8-10]. Moreover, in a subset of GBMs, EGFR harbors an activating deletion, generating a receptor that signals in the absence of ligand binding [11]. The activity of those tyrosine kinase receptors results in the activation of several downstream signal transduction pathways, such as phophatidylinositol-3 (PI3) kinase/AKT pathway, Ras/mitogen activated protein (MAP) kinase pathway, PLC-g/PKC pathway, and c-MYC pathway [7]. These signal transduction pathways play roles in cell proliferation, differentiation, migration, apoptosis, and cell shape [12]. In addition, the tumor suppressor phosphatase with homology to tensin (PTEN) is commonly mutated or deleted in GBMs [13,14]. PTEN gene product negatively regulates the activation of AKT [15], a protein with both antiapoptotic and mitogenic functions [16]. Therefore, signaling through the PI3K/AKT pathway would be greatly facilitated by PTEN deletion. Furthermore, inactivation of cell cycle arrest pathways is thought to be involved in GBMs formation. It has been reported that INK4a-ARF is deleted or inactivated in 60% of GBMs [17,18]. The INK4A-ARF locus encodes two important tumor suppressors, p16INK4A and p14ARF. The former prevents the phosphorylation of RB by inhibiting the cyclin-dependent kinase 4 (CDK4) or 6 (CDK6)-cyclinD1 complex [19]. The phosphorylation of RB would result in the release of transcription factor E2F and subsequent transcription of proteins necessary for cell cycle progression. The other tumor suppressor, p14ARF protects the important tumor suppressor p53 from MDM2-mediated degradation [20]. p53 leads the cells to either cell cycle arrest or apoptosis depending on cell type and stimuli [21]. The remaining tumors frequently have p53 mutation and loss of the remaining p53 allele, as evidenced by heterozygosity of chromosome 17 [22]. If neither p14ARF nor p53 is directly affected, p53 may still be inactivated by MDM2 amplification and overexpression [23].

Over the past decade, our knowledge of the causative abnormal signaling pathways involved in the glioma initiation and progression has dramatically increased as described above. However, discovery of the signaling molecules leading to formation of gliomas would greatly assist in the development of new therapeutic treatments.

Many fundamental processes in cancer cells are regulated by protein phosphorylation. Protein kinases regulate the cell's response to DNA damage, control DNA replication, regulate entry into, passage through and exit from mitosis, and ensure accurate chromosome segregation. They monitor nutrient availability, control cell growth pathways, regulate apoptosis and hence govern a cell's commitment to either live or die.

In metazoans, there are three highly related Aurora kinases: Aurora-A, B and C (FIG. 1, reviewed in [24-28]). Despite their relatedness at the sequence level, the localization and functions of Aurora-A and B are largely non-overlapping. Aurora-A is localized at the centrosome from the time of its duplication to mitotic exit and regulates centrosome function. In contrast, Aurora-B is localized to inner-centromeric chromatin from prophase until the metaphase-anaphase transition, whereupon it is relocalized to both the microtubules in the spindle midzone (and the cell cortex at the cleavage furrow) during telophase, ending up in the midbody throughout cytokinesis [24-26,28] (FIG. 2). Such behavior marks out Aurora-B as a canonical “chromosome passenger” protein [29], whose function is to ensure accurate chromosome segregation and timely cytokinesis. The approximate execution points of Aurora-A and B are shown in FIG. 2. Aurora-C's regulation and localization have remained obscure until recently [30,31] and its function remains unknown.

Among Aurora kinases, Aurora-A kinase has been well known as an oncogene. Aurora-A has been found to be amplified and/or overexpressed in various human cancers including breast, colorectal and prostate cancers and is considered to be a potential oncogene [24]. However, the roles of Aurora-A in gliomagenesis are largely unknown.

Overexpression of Aurora-A is correlated with tumor progression. Furthermore, rodent fibroblasts overexpressed with Aurora-A could form colonies in soft agar and form tumor in immunodeficient animals [33]. Thus Aurora kinase can function as an oncogene, an idea further strengthened by its interaction with tumor suppressors like p53 and BRCA1. Although abnormal mitosis caused by Aurora-A overexpression may explain the chromosome instability which is often found in cancer cells, this does not explain why rodent fibroblasts with the overexpression of Aurora-A can form colonies in soft agar. Colony formation of rodent fibroblasts in soft agar is not caused by the abnormal mitosis but caused by G1 checkpoint abrogation due to the activation of an oncogene.

We have recently found that overexpression of Aurora-A results in the activation of NFκB pathway which is known to be involved in gliomagenesis [5] (FIG. 3). The mammalian NFκB family is a sequence-specific transcription factor that is known to be involved in a variety of biological functions, including innate and adaptive immunity, inflammation, and cell survival [34]. The family of NFκB proteins include RELA (p65), NFκB 1 (p50; p105), NFκB 2 (p52; p100), c-REL and RELB [35]. They form various NFκB homo- and hetero-dimers, all of which are predominantly found in the cytoplasm complexed with the IκB family of inhibitory molecules [35]. Degradation of IκB proteins in response to stimuli is an obligatory step in NFκB activation [36]. This degradation is mediated by the proteasome and requires that the IκB molecules are phosphorylated at specific residues by IκB kinases (IKK₁ and IKK2) [37][38].

Although each NFκB dimer is likely to have distinct regulatory functions, many of the target genes are common to several NFκB proteins. These genes fall into four broad functional categories: immunoregulatory and inflammatory genes; anti-apoptotic genes; genes that positively regulate cell proliferation; and genes that encode negative regulators of NFκB. It is worth noting that genes in these four categories can contribute to tumorigenesis. Moreover, NFκB activation was found to stimulate angiogenesis—a process that requires both migratory and invasive capacities of vascular epithelial cells and plays an important role in tumor progression, possibly by inducing expression of IL-8 and vascular endothelial growth factor (VEGF) [39]. In addition, κB sites were identified in the promoters of genes that encode several matrix metalloprotease (MMPs)—proteolytic enzymes that promote glioma invasion of surrounding tissue [40]. Thus, constitutive activation of NFκB has been thought to be correlated with oncogenesis (gliomagenesis) [5]. However, the mechanism of how NFκB is activated in gliomas is poorly understood.

Current models for oncogenesis and tumor studies are based on either transplantation of cultured human or rodent brain tumor cells into often immunodeficient recipient animals or. induction of de novo brain tumors in rodents with mutagens, such as nitrosourea [41,42]. Unfortunately, both models have certain drawbacks. For instance, the xenograft or allograft models are highly reproducible, but they do not recapitulate the infiltrative characteristics, especially glial tumors and remain a “metastatic” model [43]. In addition, strong selective pressure is seen in all forms of cell culturing, raising concern that cells used for transplantation experiments may not faithfully represent the original tumor. Conversely, tumors induced by mutagenic agents grow within brain tissue and histologically resemble gliomas. However, because the lesions are caused by unknown mutations, the genetic profile is unknown and differs between tumors, thus making them nonreproducible [41]. Transgenic mice that have expression of an oncogene driven by GFAP or Nestin promoter and knockout mice that have targeted deletions in tumor suppressor(s) are often used for modeling brain tumors in mice. However, tumors in these mice often arise as a result of secondary mutations, made possibly by the original alteration(s) during the development of these mice. Somatic cell gene transfer using retroviral vectors to the neonatal mouse brain is the other method to model brain tumors in mice. Especially, an avian leukosis virus-based replication-competent system can induce oncogene(s) in a cell type specific manner [44]. Avian leukosis virus-based RCAS vectors are injected into the brain of transgenic mice expressing the RCAS receptor, tv-a, on target cells. In the case of brain tumors, transgenic mouse lines expressing the tv-a receptor from GFAP or nestin promoter are generated and used for this system. An elegant study using this system demonstrated that combined activation of Ras and AKT signaling in glial progenitor cells is enough to induce glioblastoma-like lesion [44]. However, as discussed above, immature progenator cells may not be representative subjects for the study of tumors in adults. Furthermore, most preclinical studies for the new strategies of the treatment for GBMs have been utilizing xenograft model using immunodeficient recipient animals. However, the result from this model does not often correlate with what actually happens in humans.

Recently a variety of Aurora kinase inhibitors, such as VX680, hesperidin and ZM447-439 have been used in clinical trials in patients with malignant solid tumors [45], although the precise mechanism of how Aurora-A inhibitors kill tumor cells remains unknown. Among these inhibitors, VX-680 has broad and potent antiproliferative activity against a variety of cell lines in vitro and robust antitumor activity in animal models bearing xenografts of tumors [46]. However, activities of the drug in athymic mouse xenograft models do not reliably predict activities in actual human tumor [45]. Moreover, it has been shown that the inhibition of Aurora-A in human cells causes various mitotic failure which results in the chromosome instability [47]. Therefore, inhibitors of Aurora-A may cause an additional chromosome instability in cancer cells or dividing normal cells which may lead to the generation of other types of cancer. Thus, it would be valuable to examine how Aurora-A inhibitors affect tumor progression in more representative tumor models.

Another troublesome area in which new tumor models are desirable is in pancreatic cancer. Thirty thousand Americans are diagnosed every year with pancreatic cancer and 30,000 die of this disease (84-86). There are no known therapies once the tumor is not resectable and has spread-unfortunately it is usually in the advanced stage when the patient sees a physician, by which time there is no recourse. Pancreatic cancer remains one of the most lethal human cancers, and 50 years of conventional treatments like chemotherapy, surgery and radiation have had little impact on the aggressive course of the disease that is inevitably fatal. Nearly all the patients with pancreatic cancer develop metastasis and die because of the debilitating metabolic effects of their unrestrained growth. The poor prognosis is likely due to detection of the disease at an advanced stage, by which time it is highly refractory to currently available cancer therapies. Therefore it is imperative to find new therapies and drugs.

The pancreas is a common site for development of early noninvasive clonal epithelial expansions termed pancreatic intraepithelial neoplasia (Panln), extremely common in elderly persons (87). In a minority of cases Panlns serially acquire genetic changes that can lead to invasive adenocarcinoma. The malignancy is thought to arise from the pancreatic ducts (exocrine cells) on the basis of its histological and immunohistochemical relationship to this cell type (88). In keeping with the general theme of acquisition of either gain of function (oncogenes) or loss of function (tumor suppressors), there appear to be both events occurring in the progression towards end stage metastatic pancreatic ductal adenocarcinoma.

Although the there are no treatments known to delay or prevent the spread of pancreatic cancer, the genetic alterations leading to pancreatic cancer are relatively well defined in that over 90-95% of cases have activating mutation in KRAS^{G12D} and inactivating mutations in the cell cycle inhibitor Ink4/p16 locus. (See, Table 2 in ref 86) Additionally 75% of cases have inactivating p53 mutations and an additional 55% show mutations in SMAD4 that effects the TGF-β pathway and perhaps angiogenesis. Additionally it has also been shown that in most pancreatic cancers transcription factor NFkB is constitutively active. In some tumors (about 10%) mutations in BRCA2 and DNA mismatch repair genes MSH2 and MLH1 have also been reported (92-93).

Tumor models made previously for pancreatic cancer exhibit invasive macroscopic tumors with long latency (5 months) and in the second case locally invasive cancers with a short latency (10 weeks median survival) have been obtained as described below in more detail.

The genetic alterations in the pancreatic cancers have been cleverly exploited to generate mouse models of pancreatic ductal adenocarcinoma. Using cytokeratin 19 promoter to drive the expression of activated Kras in differentiated pancreatic ductal epithelium, transgenic mice were generated which did not develop overt neoplastic lesions of the ducts but did develop PanIn (94, 95). Similarly mice with constitutive deletion of both or either component of the Ink4a/Arf locus do not develop spontaneous pancreatic adenocarcinoma (96-99). Ron DePinho and colleagues designed experiments to assess the cooperative interactions of these two mutations in mice (100). They engineered mice where mutant Kras allele (Kras^{G12D}) was activated and Ink4/Arf deleted by using pancreas-specific CRE recombinase resulted in an earlier appearance of PanIn lesions. More importantly these intraepithelial neoplasams rapidly progressed to highly invasive and metastatic cancers, resulting in death in all cases by 11 weeks (100). Recently, Hingorani et al (101) have generated transgenic mice with concomitant endogenous expression of Trp53^{R172H} and KraS^{G12D} in the pancreas, which shows invasive and widely metastatic pancreatic ductal adenocarcinoma, reminiscent of the human condition. In contrast to the highly invasive local tumors seen in mice with concomitant activation of oncogenic Kras and inactivation of Ink4/Arf, these mice develop macroscopic metastasis to organs also affected in human disease. A remarkable feature of these tumors was the widespread chromosomal instability with aberrant chromosomal rearrangements and nonreciprocal translocations with apparently intact telomeres (101, 102).

Mouse models of pancreatic cancers have faithfully mimicked the human disease genetically and morphologically, a testament to the usefulness of mouse models for human cancer (103). However, it is clear that mere activation of oncogenic Kras, while sufficient to cause invasive and metastatic pancreatic adenocarcinoma is not a sufficient model because it takes a very long time (94,95). Combining Kras with deletion of Ink4/Arf speeded up the tumor incidence and median survival of only 8 weeks, but neither macroscopic metastases in other organs nor genetic instability typical of human tumors was observed (100) in these models. A combination of Kras and dominant negative TP53 (R172H) showed macroscopic metastases in many organs and chromosomal instability but the median survival was 5 months (101). Though 95% of all pancreatic cancers have Kras mutations as the initiating event, it appears that only one-third of all pancreatic tumors have mutations in all three of the suppressor genes (CDKN2/INK4/A/p16, TP53 and SMAD4), while an additional one-third cancers have mutations in two out of three tumor suppressor genes. A consensus report on the pathology of genetically engineered mouse models for pancreatic cancer has recently been compiled (104). Although the technology of generating mice with conditional lethal genes has been very valuable in studying tumorigenesis, it is not without pitfalls. The most obvious one being that crossing with mice containing tissue specific CRE forces the activation or deletion of the intended gene in every cell in that organ. Human tumors do not occur by activating oncogenes or inactivating suppressor genes in every cell, but most likely initiate in few cells, which presumably stochastically accumulate mutations to become full blown invasive and metastatic tumors. Mutated cells are usually surrounded by genotypically normal cells and not by cells mutated in Kras, p16 and/or p53. Thus the tumor microenvironment is conceivably altered in ways that do not recapitulate normal tumor progression as these key regulators undoubtedly participate in important cellular regulatory processes. This is particularly noteworthy as in recent years the tumor microenvironment has been shown to have important functions in the tumorigenic process (105).

What is still needed is a mouse model with activated Kras, and inactivated p53, p16 and Smad4. It would be additionally beneficial to have a model in which only a few cells (rather than the whole organ) in the pancreas are manipulated such that Kras is activated and p16, p53, and Smad 4 are inactivated. Such a model in which the cells progress to invasive, rapidly growing, macroscopic tumors with chromosomal instability would be extremely helpful to the study of pancreatic cancers and treatments therefore.

In view of the above, there is an urgent need to develop models of tumors that correlate better with tumors in adult animals and, particularly, human patients. There is a need for novel strategies to identify molecular targets to test treatment using tumor models that are more predictive than conventional models. There is a need to model tumors of all types under conditions that recapitulates the pathobiology of human tumorigenesis. With regard to GBMs and pancreatic cancer in particular, there is a particular need for representative models, e.g., mouse models, to screen potentially beneficial therapeutic peptides and small molecules. The present invention provides these and other features that will be apparent upon review of the following.

### SUMMARY OF THE INVENTION

The present inventions are directed to compositions and methods of generating localized foci of model tumor cells highly correlated to actual clinical tumor cells including, but not limited to, glioblastomas and pancreatic cancer tumor cells. Lentiviral vector particles are engineered and injected into a target cell animal host to transduce target cells into tumor model cells. The characteristics of the tumor cells can be influenced by the peptides to be expressed from the vector, the choice of target cell animal, the choice of target cell type, the choice of a point of vector particle injection into an organ, the incubation period of transformed cells, and selection of desired model cells in primary culture and cloning. Optionally, tumor model cells can be implanted into an animal of choice at a location of choice to provide a model animal for in vivo studies of the tumor. The choice of options at each step provides a degree of control over the characteristics of the model systems unknown in the prior art. For example, the tumors generated with the methods provided herein mimic the human course of disease, e.g., in being rapid, invasive and highly metastatic.

Unique nucleic acid expression vectors can be employed in the methods of providing highly correlated tumor models. For example, an expression vector of the invention can include a sequence comprising a first lox recombination site and a second lox recombination site bracketing a nucleic acid sequence encoding a stuffer sequence, a promoter sequence upstream from the lox recombination sites, a coding sequence of interest and a first marker nucleic acid sequence encoding a first marker, each downstream from the lox recombination sites, and a lentiviral packaging site (psi). The vector can be packaged into a lentiviral vector particle using a packaging host cell that comprises the recombinant nucleic acid, a lentiviral packaging plasmid that encodes a lentiviral gag protein and a pseudotyping plasmid that encodes an envelope protein that is heterologous to the lentiviral gag protein. It is preferred that the vector particle is a pseudotyped lentiviral vector, which packages the recombinant nucleic acid in a lentiviral capsid, e.g., including an external Env protein heterologous to other proteins of the vector particle.

In preferred embodiments the recombinant nucleic acid comprises sequences in the order of promoter, first lox recombination site, stuffer, second lox recombination site, sequence of interest and first marker. The promoter can be a eukaryotic constitutive promoter (e.g., a CMV promoter) or an inducible promoter. The stuffer sequence preferably encodes a second marker or a stop codon. The first and/or second markers are preferably fluorescent proteins, such as, e.g., RFP (red fluorescent protein) or GFP (Green fluorescent protein). The sequence of interest can preferably encode, e.g., an oncogenic polypeptide or an anti-tumor suppressor moiety. For example, the encoded oncogenic polypeptide can be a ras protein, myc, src, AKT, Aurora-A kinase, BAFF, Kras, a peptide that represses expression or activity of a tumor suppressor (e.g., p53, Smad4, Ink4/p16, or the like), a serine kinase, a G-protein, a threonine kinase, and a tyrosine kinase. Representative anti tumor suppressor moiety can include, e.g., an antisense DNA, an RNAi that inhibits expression of a tumor suppressor, an siRNA that inhibits expression of a tumor suppressor, and/or the like.

Target cells transduced with the vectors of the invention are an aspect of the invention. For example, the target cells of the invention can be cells in organs of a target cell host animal transduced with vector particles of the invention to include the recombinant expression vector nucleic acids of the invention. In an embodiment, the target cells express a GFAP+ (glial fibrillary acid protein positive), CD133−, and nestin+ phenotype before transduction. In optional embodiments, the target cell expresses Cre recombinase. The target cells can be part of an organ or tissue of a living animal, or derived from an animal, e.g., a neuronal cell, a pancreatic cell, a prostate cell, a breast cell, a liver cell, a skin cell, a differentiated cell, and a mature astrocyte or a lung cell.

The present inventions include non-human recombinant laboratory animals transduced with the vectors of the invention. For example, tumor model animals of the invention can include laboratory animals harboring model cells with a recombinant Cre recombinase and a recombinant nucleic acid that encodes a sequence comprising a first lox recombination site and a second lox recombination site bracketing a nucleic acid sequence encoding a stuffer sequence, or a recombinant nucleic acid that encodes a sequence comprising recombined lox recombination sites, a promoter sequence upstream from the recombined lox recombination sites, a coding sequence of interest and a first marker nucleic acid sequence encoding a first marker, each downstream from the recombined lox recombination sites, and a lentiviral packaging site (psi). The animal can be any appropriate choice to affect the model cell character and facilitate growth of the model tumor, e.g., a mouse, a rat, a monkey or the like.

As with the model cells, model animals can express a phenotype, such as GFAP negative, nestin positive, CD133 negative, p53 negative, p16 negative, and/or the like. Model cells in the animal can be, e.g., brain cells, pancreatic cells, prostate cells, breast cells, lung cells, liver cells, and the like. The cells can express markers, such as red fluorescent protein or green fluorescent protein to provide, e.g., information on the transduction or gene expression status of the cells. Model cells in the model animals can express a coding sequence of interest, such as, e.g., ras, myc, src, AKT, Aurora-A kinase, BAFF, Kras, a sequence coding for an agent that represses expression or activity of a tumor suppressor (such as tumor suppressors p53, Smad4, Ink4/p16, and the like), a sequence encoding a serine kinase, a sequence encoding a G-protein, a sequence encoding a threonine kinase, and a sequence encoding a tyrosine kinase; thus selectively affecting the phenotype of the model cells.

Model systems of the invention can be employed in methods of screening for modulators of tumorigenesis and modulators of model cell characteristics. The methods can include providing a cell or non-human laboratory animal expressing Cre. The cells or animals can be, e.g., transduced with a recombinant nucleic acid comprising a first lox recombination site and second lox recombination site bracketing a nucleic acid sequence encoding a first marker, a promoter upstream from the lox recombination sites, a coding sequence of interest and a nucleic acid sequence encoding a second marker each downstream from the lox recombination sites. Expression of the sequence of interest can contribute to changes in tumor-associated characteristics to influence tumorigenesis and/or a tumor phenotype in the cell or animal. In methods of screening for modulators, the cell or animal can be contacted with a putative modulator of tumorigenesis or the tumor phenotype and changes in tumor-associated parameters can be detected in the cell or animal. Modulators can be selected that modulate characteristics of the tumor model cells on contact.

The methods of screening for modulators include steps in the preparation of vectors and model cells. For example, the methods include packaging the lox sites, marker sequences and coding sequence into a vector particle in a packaging cell. The packaging cell can include the recombinant nucleic acid, a lentiviral packaging plasmid that encodes a lentiviral gag protein and a pseudotyping plasmid that encodes an envelope protein that is heterologous to the lentiviral gag protein.

Model cells used in methods of screening can be those described above. For example, the cell can be a transduced astrocyte from an adult animal, a brain cell, neuronal cell, a pancreatic cell, a prostate cell, a breast cell, a liver cell, a skin cell, a differentiated cell, a lung cell and/or the like. The transformed model cells can have a phenotype of, e.g., GFAP negative, nestin positive, CD133 negative, p53 negative, and/or p16 negative. A coding sequence of interest in the model cells can be, e.g., ras, myc, src, AKT, Aurora-A kinase, NFκB, BAFF, Kras, a sequence coding for an agent that represses expression or activity of a tumor suppressor, (e.g., p53, Smad4, Ink4/p16, or the like), a sequence encoding a serine kinase, a sequence encoding a G-protein, a sequence encoding a threonine kinase, a sequence encoding a tyrosine kinase, and/or the like.

Putative modulators of the model cell characteristics can be selected from any molecules that can be manipulated to functionally contact the model cells in their environment. For example, putative modulator can be an anti-cancer drugs, VX680, hesperidin, ZM447-439, a molecule from a small molecule library, and the like. Putative modulators can be selected as modulators if they detectably change a tumor associated parameter of the contacted model cells (e.g., as compared to appropriate contact controls). For example, a modulator can be selected on detection of increased cellular density in the model cells on contact, necrosis, invasion of surrounding tissue by the cells, nuclear pleomorphism, giant cell formation, increased mitotic cells, lack of contact inhibition, increased nucleus to cytoplasm ratio, and/or other tumor-associated characteristics relevant to the tumor being modeled.

The methods of the invention include treatment of a disease state in the animal by transducing cells of the animal with vector particles comprising coding sequences of interest that result in a diminution of tumor-associated parameters in tumor cells of a clinical patient receiving the vector particles. For example, the tumor of a patient can be injected with lentiviral vector particles comprising one or more sequence encoding a suppressor of an oncogene or one or more sequences encoding a therapeutic polypeptide.

An important aspect of the present invention is methods providing the ability to generate transduced cells and/or model tumor cells at a localized foci. The methods allow specific transduction of particular cells and/or their growth in a specific selected environment. The methods allow the foci tumors to grow without interference from effects of independently generated tumors that often occur in tumors generated using old art techniques.

Method of generating localized foci of transduced cells in vivo can include providing a vector system, e.g., a lentiviral vector system, comprising one or more recombinant nucleic acid expression vector as described above, e.g., including an encoded oncogenic sequence of interest. The methods further include providing an animal and injecting the vector into an organ of the animal at a desired location. Target cells at the location can be transduced with the recombinant nucleic acid by the vector system to generate a localized foci of transduced target cells in the organ.

The vector system can be placed in contact with target cells using any appropriate method or devices. Typically, the vector system particles are injected to proximity of the desired target cells by inserting the tip of a needle to the desired location and depositing the lentiviral vector from the needle tip. High efficiency has been obtained in many of these injections and injection of 10⁸ IU or less of the lentiviral vector can locally transduce or transform 100 or less cells at a foci. Methods of the invention include generation of transduced cell foci at a desired location without generation of tumors in other organs or tissues of the animal.

Optimal cells for production of model cells highly correlated to a clinical tumor of interest can be empirically discovered by testing transductions at various organ and tissue locations. Optionally, knowledge about desirable cell types can direct the choice of vector particle injection for target cell contact. For example, locations for transduction and resultant transformations can be screened by injecting the vector at a plurality of test locations in an organ or tissue, and selecting a location resulting the highest transduction efficiency or selecting a location providing a desired change in transduced cell phenotype.

In summary, the mouse models and methods for producing them provided herein have genetic alterations associated with human cancers, including, but not limited to, GBM, pancreatic and lung cancer. In addition, tumors generated with these methods mimic the human disease course, e.g., in being rapid, invasive, and highly metastatic in the case of pancreatic cancer. Such mice are highly valuable for testing cellular therapies and novel drugs, for instance avastin, which blocks angiogenesis and may be able to slow the progression of the disease.

### DEFINITIONS

Unless otherwise defined herein or below in the remainder of the specification, all technical and scientific terms used herein have meanings commonly understood by those of ordinary skill in the art to which the present invention belongs.

Before describing the present invention in detail, it is to be understood that this invention is not limited to particular methods, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a component” can include a combination of two or more components; reference to “a sequence” can include combinations of sequences, and the like.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “about” as used herein indicates the value of a given quantity varies by a non-critical amount in the context of the parameters at issue, e.g., +/−10% of the value, or optionally +/−5% of the value, or in some embodiments, by +/−1% of the value so described.

The term “polynucleotide” (and the equivalent term “nucleic acid”) encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or polymers of nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded. A recombinant nucleic acid is a nucleic acid sequence that includes sequences combined by the hand of man in the laboratory, e.g., using standard genetic engineering techniques known in the art.

The phrase “non-dividing” cell refers to a cell that does not go through mitosis. Non-dividing cells may be blocked at any point in the cell cycle, (e.g., G₀/G₁, G₁/S, G₂/M), as long as the cell is not actively dividing. For ex vivo infection, a dividing cell can be treated to block cell division by standard techniques used by those of skill in the art, including, irradiation, aphidocolin treatment, serum starvation, and contact inhibition. However, it should be understood that ex vivo infection is often performed without blocking the cells since many cells are already arrested (e.g., stem cells). The recombinant lentivirus vectors used in methods and compositions of the invention are typically capable of infecting non-dividing cells, regardless of the mechanism used to block cell division or the point in the cell cycle at which the cell is blocked. Examples of pre-existing non-dividing cells in the body include mature differentiated neuronal, muscle, liver, skin, heart, lung, and bone marrow cells, and their derivatives.

A “polynucleotide sequence” or “nucleotide sequence” is a polymer of nucleotides (an oligonucleotide, a DNA, an RNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

The terms “cell proliferative disorder” or “cellular proliferative disorder” refer to any disorder in which the proliferative capabilities of the affected cells is different from the normal proliferative capabilities of unaffected cells. An example of a cell proliferative disorder is neoplasia. Malignant cells (e.g., cancer cells) develop as a result of a multistep process. The term “cancer” as used herein, includes any malignant tumor including, but not limited to, carcinoma and sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the spread of a malignant tumor from its sight of origin. Cancer cells may metastasize through the bloodstream, through the lymphatic system, across body cavities, or any combination thereof. The term “cancerous cell” as provided herein, includes a cell afflicted by any one of the cancerous conditions provided herein. The term “carcinoma” refers to a malignant new growth made up of epithelial cells tending to infiltrate surrounding tissues, and to give rise to metastases. The term “clinical” with regard to tumors and tumor cells refers to tumors that were not generated by the hand of man in the laboratory, but are naturally occurring in humans or animals.

A promoter is a DNA sequence that contains information, in the form of DNA sequences, that permits the proper activation or repression of the gene which it controls, i.e. whether RNA is synthesized or not. The promoter contains specific sequences that are recognized by proteins known as transcription factors, such as RNA polymerase. A promoter controls transcription of sequences “downstream” from the promoter, i.e., in the direction of transcription by RNA polymerase.

A coding sequence of interest is a sequence encoding a polypeptide that is intended to be translated to affect the phenotype of a transduced target cell. Typically, a marker polypeptide sequence is not considered to be the coding sequence of interest in vectors of the present invention.

A packaging cell is a host cell for the vector system, e.g., a lentiviral or other viral vector system. The packaging cell is typically transfected with nucleic acids (e.g., plasmids) encoding the components comprised in the vector particle (e.g., recombinant nucleic acid of interest, a reverse transcriptase, a lentiviral packaging plasmid that encodes a lentiviral gag protein and a pseudotyping plasmid that encodes a envelope protein that is heterologous to the lentiviral gag protein).

A protein heterologous to another protein, in the context of the present inventions, refers to the fact that the two proteins have sequences normally found in different species of life form, such as a first protein from HIV and a second protein from RSV.

A stuffer sequence is a nucleic acid sequence between 2 lox recombination sites, which is excised due to Cre recombinase activity. The stuffer sequence may encode a polypeptide, or not. In various preferred embodiments herein, the stuffer sequence encodes a marker polypeptide that can easily be detected or screened for, such as a fluorescent protein.

The term “neoplasm” refers to a new, abnormal growth of cells or a growth of abnormal cells that reproduce faster than normal. A neoplasm creates an unstructured mass (a tumor) which can be either benign or malignant. For example, the neoplasm may be a head, neck, lung, esophageal, stomach, small bowel, colon, bladder, kidney, or cervical neoplasm. The term “benign” refers to a tumor that is noncancerous, e.g. its cells do not proliferate or invade surrounding tissues. The term “malignant” refers to a tumor that is metastatic or no longer under normal cellular growth control. Examples of cancers include colon cancer, lung cancer, breast cancer, prostate cancer, and melanoma, amongst others.

Tumorigenesis, as used herein, refers to the steps in generation of a tumor from transformation of a non-tumor cell to a tumor cell, to growth of a tumor, metastasis and growth of two or more tumors at new locations in an animal. The model cells of the invention provide models at each step of tumorigenesis that can correlate to tumorigenesis in the clinical progress of a tumor in a human or animal individual.

The term “patient” or “subject”, as used herein, refers to any individual to which the subject methods are performed. Generally the subject is human clinical patient, although as will be appreciated by those in the art, the subject may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. Non Human “laboratory animals” are those that are typically used in cancer models, including rodents (mice, rats, rabbits) and primates such as macaques.

With regard to preparing animal models for tumors, the term “seeding” refers to implantation of one or more model tumor cells into a host animal.

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician.

The term “pharmaceutically acceptable”, when used in reference to a carrier, is meant that the carrier, diluent or excipient must be compatible with the other ingredients of the formulation and not significantly deleterious to the recipient thereof.

The terms “administration” or “administering” is defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration. Oral administration is taken by mouth into the digestive tract. Administration by inhalation is can include inhalation of a mist or powder into the nasal passages, upper respiratory tract or lungs.

As used herein “corresponding normal cells” means cells that are from the same organ and of the same type as the cells being examined. In one aspect, the corresponding normal cells comprise a sample of cells obtained from a healthy individual. Such corresponding normal cells can, but need not be, from an individual that is age-matched and/or of the same sex as the individual providing the cells being examined.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, CSF, semen, and urine.

### BRIEF DESCRIPTION OF THE DRAWINGS

FIG. 1 is a schematic diagram depicting the structure of human Aurora kinases. The catalytic domain of Auroras-A, B and C is highly conserved. Autophosphorylation of Thr288 in the activation loop of Aurora-A is required for the activation of its kinase activity. A short amino acid peptide motif called the ‘destruction box (D-box)’ is present in the carboxy-terminal region of Auroras-A, B and C. The D-box is recognized by adaptors of the anaphase-promoting-complex/cyclosome (APC/C) and thereby targets these proteins for degradation via the ubiquitin/proteasome dependent pathway.

FIG. 2 shows diagrams of subcellular localization of Aurora-A and Aurora-B during the cell cycle: Aurora-A (dots) is first detected at the centrosome during G2 phase. The activation of a small proportion of Aurora-A at centrosomes was first evident before chromatin condensation at late G2 phase. Both the amount and activity of Aurora-A rapidly increase in the centrosome, and a fraction of active Aurora-A subsequently translocates into nucleus coincident with chromatin condensation at prophase. After nuclear envelope breakdown (NEBD), activated Aurora-A is observed at the spindle poles and bipolar spindles during prometaphase and metaphase. The amount of Aurora-A starts to decrease at the metaphase-anaphase transition, but a small fraction of Aurora-A remains on the centrosomes and the spindles at the onset of anaphase and localizes on central spindle and centrosomes at late anaphase. Aurora-A is degraded by the Cdhl/Fizzy-related form of the anaphase-promoting complex/cyclosome (APC/C). At the final stage of cytokinesis, most of the Aurora-A protein becomes undetectable. Aurora-B (squares) is a chromosome passenger protein, localizing on chromosome arms during prophase and the interface between sister centromeres (inner centromere region) during prometaphase and metaphase. It then relocalizes to the spindle midzone and the cell cortex, at the site of cleavage-furrow ingression in late mitotic phase. During cytokinesis, Aurora-B localizes at midbody. It has been recently shown that Aurora-C is also a chromosome passenger protein that co-localizes with Aurora-B.

FIG. 3 shows a chart showing a relationship between Aurora-A expression and IkBaM. 293T cells were transfected with Aurora-A expressing plasmids and kB reporter plasmids with or without IkBaM expressing plasmids. At 24 hrs after the transfection, equal amounts of cell lysates were used to detect the luciferase activity.

FIG. 4 shows an example of a transduction according to methods of the invention. The expression of Flag H-RasV12 was clearly induced in the presence of Cre recombinase in vitro. FIG. 4A is a diagram of pTomo-H-RasV12 lentiviral vector. The stuffer fragment, red fluorescent protein (RFP), keeps the translation of Flag H-RasV12 (activated form of H-Ras) in an OFF state. The excision of the stuffer sequence by Cre recombinase results in the expression of Flag-tagged H-RasV12. Expression of RFP was dramatically reduced in the presence of Cre recombinase when HeLa cells were infected with pTomo H-RasV12 lentiviral vectors (MOI: 10) with or without pBOB CRE lentiviral vectors (MOI: 10) that can express Cre recombinase (data not shown). FIG. 4B shows Flag H-RasV12 expression was successfully induced by Cre recombinase. HeLa cells were infected with pTomo mock lentiviral vectors (lanes 1 and 2, MOI: 10) or pTomo H-RasV12 virus (lanes 3 and 4, MOI: 10) with (lanes 2 and 4) or without (lanes 1 and 3) pBOB CRE lentiviral vectors (MOI: 10) that can express Cre recombinase. Two days after the infection, total cell lysates were prepared and subjected to western blotting using anti-Flag antibodies and anti-Cre antibodies. β-actin detection was used as a loading control. In FIG. 4A, CMV represents a human cytomegalovirus promoter, IRES an internal ribosomal entry site and GFP represents a green fluorescent protein sequence.

FIG. 5 is a schematic diagram of the locations injected with lentiviral vectors. A sagittal view of an adult mouse brain is shown in FIG. 5A. Arrows show the locations (i: cortex, ii: subventricular zone, iii: hippocampus subgranular zone) where the injections of lentiviral vectors were performed. pTomo H-Ras V12 lentiviral vectors were successfully injected into the locations injected. Five days after the stereotactic injection of 0.8 μl of pTomo H-Ras V12 lentiviral vectors (1×10⁸ IU) into cortex, hippocampus, and subventricular zone in GFAP Cre mice, brains were perfused, fixed and sectioned (data not shown). The sections were observed by confocal microscopy. GFP signals indicates the cells infected with pTomo H-RasV12 lentiviral vectors. Merged images with nuclear staining with DAPI are shown in panels b, d and f. In FIG. 5B, numbers of the cells infected with pTomo H-Ras V12 lentiviral vectors in each location (black bars) are shown. Numbers of GFAP positive cells infected with the vectors are shown in lighter bars.

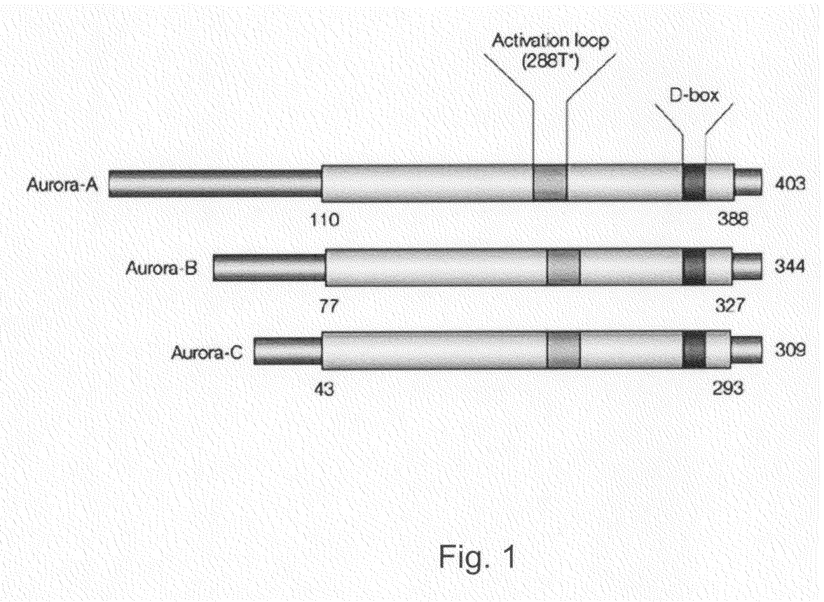

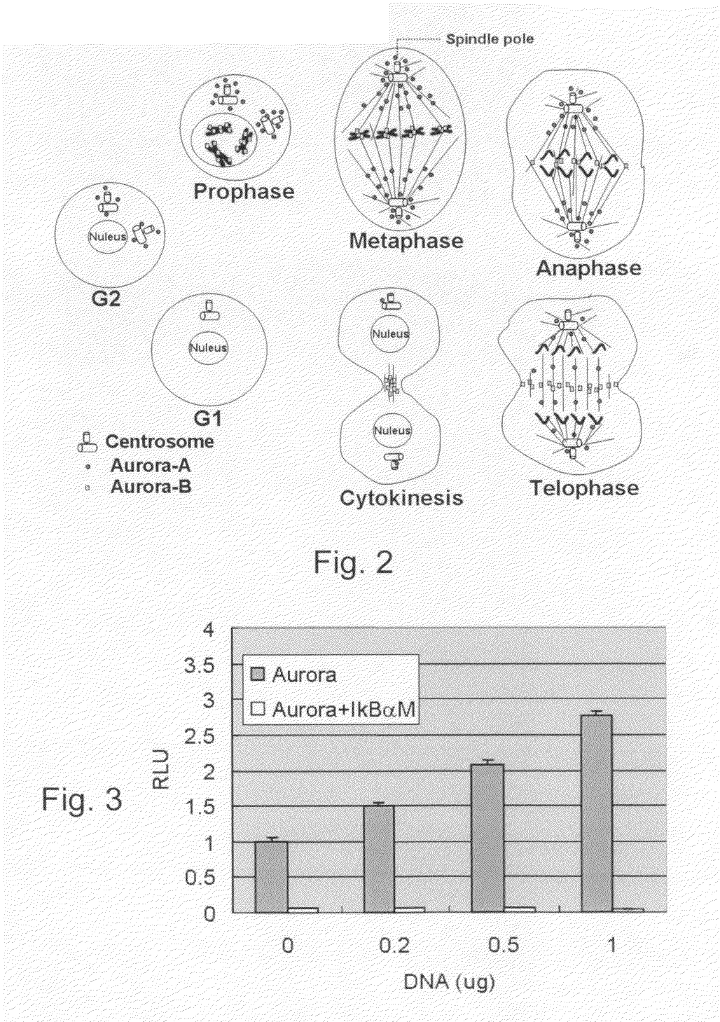

In FIG. 6, Flag H-RasV12 is induced specifically in GFAP positive cells of adult GFAP Cre mice. Cre recombinase is specifically expressed in GFAP positive cells, as shown in FIG. 6A. Brain sections from adult GFAP Cre mice at the age of 8 weeks were stained with Cre (panels a and d), GFAP (panel b) and NeuN (panel e). Merged images are shown in panels c and f. DAPI (panel c) was used to indicate nucleus. In FIG. 6B, Flag RasV12 was successfully induced in the subgranular zone of the hippocampus in GFAP-Cre mice but not in wild type mice. Seven days after the injection of pTomo H-RasV12 vectors into hippocampus in wild type (panels a-d) or GFAP Cre mice (panels e-h), the mice brains were removed, fixed and sectioned and stained with anti-Flag antibodies (panels c and g; magenta) and DAPI (panels d and h) to visualize DNA, and analyzed by confocal microscopy. GFP signals are shown in panels a and e, and RFP signals are shown in panels b and f. Merged images are shown in panels d and h. In FIG. 6C, Flag H-RasV12 was expressed specifically in GFAP positive cells. Five days after the injection of pTomo RasV12 into cortex, the GFAP Cre mice were euthanized and tissues fixed. Sections of the brain were stained with anti-Flag antibodies (panel a) and anti-GFAP antibodies (panel b). Merged images are shown in panel c.

FIG. 7 shows representative images of the mouse showing tumor formation. In FIG. 7A, a GFAP Cre mouse injected with pTomo H-RasV12 and pTomo AKT into right hippocampus showed enlarged head 93 days after the injection. Gross appearance of the brain suggests massive lesion in the cerebrum, as shown in FIG. 7B. The sections (40 μm) were processed for H-E staining in FIG. 7C. The dark area shows the tumor which has increased cellular density. In FIG. 7D, the sections (40 μm) were analyzed by confocal microscopy. GFP positive cells spread over the tumor.

FIG. 8 shows histological characterizations of tumors induced by the combined activation of H-RasV12 and AKT in the hippocampus of GFAP-Cre mouse. Tumor tissues were processed for H-E staining (panels A-D). Increased cell density (panel A), pseudopalisading (panel B, white arrow), necrosis within the dense cellular region (panel C, arrow head) and perivascular invasion (dotted arrow in panel D) which were human glioblastoma characters were found in this tumor, but nuclear pleomorphism was not apparent. Representative images of GFP-positive tumor tissues are shown in panels E-J. The tumor sections were analyzed by confocal microscopy. Most of the tumor cells (more than 90% in every region examined) were GFP positive (panels E-J; lighter cells). White arrows in panel F show the invasion of the GFP-positive tumor cells. The tumor contained heterogeneous area (panels G and J). DAPI staining was performed to visualize DNA in merged images (panels H-J). Immunohistochemistry of the tumors induced by H-RasV12 and AKT in GFAP-Cre mice (panels K-O). Tumor sections were stained with the antibodies against GFAP (panel K), Nestin (panel L), Flag (panel M), HA (panel N) and Ki67 (panel 0). T and N in panel K indicate regions of tumor and normal brain tissue respectively. White arrows in panel O show the positive cells for Ki67. Hematoxylin staining was also performed to visualize nucleus in panels K-O. The tumor contained trapped normal astrocytes which are positive for GFAP but negative for GFP (panels P-R). An image in panel R shows a merged image with TOTO3 staining to visualize DNA.

FIG. 9A shows the effects of p53 loss on the model system. Loss of p53 shortened the tumor latency and induced nuclear pleomorphism and high mitotic activity in the tumor induced by combined activation of H-Ras and AKT. Kaplan-Meier graph showing glioma free survival in GFAP-Cre mice (panel A) and in GFAP-Cre/p53^{+/-} mice injected with pTomo H-Ras and pTomo AKT. H-E staining of the tumor induced by combined activation of H-RasV12, AKT and loss of p53 (panels B-D). GFP positive tumor cells spread over the tumor area (panel E). T and N in panel B indicate the tumor area and normal tissue area respectively. Black arrow in panel B indicates hemorrhage found in the tumor. The black arrowhead in panel C indicates the area of necrosis. White arrows in panel D indicate giant cell formation in the tumor. FIG. 9B, panels A to E, shows tumors induced by combined activation of H-Ras and AKT in GFAP-Cre/p53+/− mice were positive for nestin but negative for GFAP. Sections from the tumor induced by combined activation of H-Ras and AKT in GFAP-Cre/p53+/− mice were stained with the antibodies against Flag (panel A), HA (panel B), GFAP (panel C), nestin (panel D), and Ki67 (panel E). White arrows in E indicate the cells positive for Ki67, which is a marker for proliferating cells.

FIG. 10 demonstrates that Aurora-A is overexpressed in glioma cell lines and tissues. Equal amounts of cell lysates from human glioma cell lines (A172, DBTRG-05MG, M059J, M059K, LN229, T98G) and mouse glioma cell lines (005 tumor and 006 tumor) were subjected to western blotting using anti-Aurora-A antibodies. Loading control was shown by α-tubulin detection. Representative images of Human glioma tissues that were stained with anti-Aurora-A antibodies and anti-p65 antibodies show that p65 is found in the nucleus suggesting that NFκB is activated in glioma tissues.

FIG. 11 is a representative image of mouse showing tumor enlarged head injected with pTomo Aurora-A. pTomo Aurora-A was injected into right hippocampus in GFAP-Cre/P53−/− mice. Three months after the injection, one out of 5 mice showed gait disturbance with enlarged head, whereas no control mice injected with pTomo mock vectors did. Black arrow indicates enlarged head.

FIG. 12 is a diagram of pTomo-BAFF. The stuffer fragment mRFP keeps the translation of BAFF in an OFF state. The excision of the stuffer sequence by Cre results in the expression of BAFF.

FIG. 13 shows immunofluorescence confocal microscopy of GFAP-Cre transgenic mouse brain. In FIG. 13A, GFP expression is shown in the hippocampus of Tomo-BAFF LV-injected mice. In FIG. 13B BAFF expression is shown in GFP- and GFAP-positive cells. Magnification: (A), ×10; (B), ×63.

FIG. 14 shows immunofluorescence confocal microscopy of Tomo-BAFF LV injected site of mouse brain. Accumulation of B220-positive B cells is seen in the brain of GFAP-Cre/p53+/− (A) or GFAP-Cre/p16+/−(B) mice. Magnification: (A), ×40; (B), ×63.

FIGS. 15A to 15F show a diagrams of plasmids useful in production of vector nucleic acids for transfection of host cells in production of lentivirus vector particles, including a PTomo mock vector.

FIGS. 16A and B show how tissue zones and cell types can be selected for transformation to provide a desired tumor model cell. FIG. 16A is a schematic diagram of regions and cell types within a mouse brain. FIG. 16B is a schematic diagram of a controlled process for production of a specific glioblastoma multiformes model tumor.

FIG. 17, panels F-H, shows that GFP positive tumor cells were GFAP negative and invaded into the normal brain. Tumor tissues were stained with anti-GFAP antibodies (panels G and H) and observed by confocal microscopy. GFP positive tumor cells invaded into normal brain tissues are indicated by white arrows in panel H.

FIG. 18 shows MRI images of a tumor induced by combined activation of H-Ras and AKT in GFAP-Cre/p53+/− mice. 73 days after the injection of H-Ras and AKT in the right hippocampus, mice were processed for MRI imaging. T1 weighted 3D FSPGR images acquired pre (panel A) and post (panel B) injection of intraperitoneal gadolinium contrast agent. Areas of abnormal contrast agent uptake are enhanced and appear bright on the image. The intensity of the tissue enhancement varies within the brain tissue (panel C); the brightest area representing the most abnormal tissue and areas with less enhancement have less tumor extensive infiltrates (panels D-F). Areas of relative intensity can be color coded to produce a 3D model of the extent and distribution of the tumor cells in the brain tissue that can be viewed from multiple aspects, from front (panel D), from below (panel E) and from side (panel F). After the MRI imaging, the mice was euthanized and processed for H-E staining of the brain, see panel G. The dark area shows the tumor area.

FIG. 19A shows the self-renewal capacity of GFP+ tumorsphere-forming cells after dissociation of primary tumorspheres and replated single-cell suspensions of these cells at serial dilutions down to one cell per well. In FIG. 19B, tumors formed in NOD-SCID mice showed histopathology of glioma-like phenotypes including the increased cellular density, necrosis within the dense cellular lesion, intratumoral hemorrhage.

FIG. 20 shows pTomo lentiviral vectors transduced in both GFAP+/nestin-cells and GFAP+/nestin+ cells. Seven days after the injection of pTomo H-Ras and pTomo AKT into the right hippocampus in GFAP-Cre/p53+/− mice, mouse brains were fixed, sectioned and stained with anti-GFAP antibodies and anti-nestin antibodies. A white arrow indicates GFAP+/nestin+ cell and a white arrow head indicates GFAP+/nestin− cell.

In FIG. 21, injection of pTomo H-RasV12 into the hippocampus in nestin-Cre mice induced the expression of H-RasV12 specifically in nestin positive cells. Panel A shows Cre was expressed specifically in nestin positive cells in nestin-Cre mice. 8 weeks old nestin-Cre mice were euthanized, and the brains were removed. The brains were fixed, sectioned and stained with anti-nestin antibodies and anti-Cre antibodies. GZ: granular zone of the dentate gyrus. A representative image of a nestin positive cell showing the expression of H-RasV12 is shown in panels B-D. 7 days after the injection of pTomo Flag H-RasV12 into the right hippocampus in 8 weeks old nestin-Cre/p53+/− mice, the brains were fixed, sectioned and stained with anti-Flag antibodies (panel B) and anti-nestin antibodies (panel C). Merged image is shown in panel C. GZ: granular zone of the dentate gyrus.

FIG. 22 shows that 005 tumor cells keep undifferentiated state even in the presence of various stimuli. Tumor cells were stimulated by forskolin and retinoic acid (panel A) to induce neuronal differentiation, BMP2 and LIF (panel C) to induce astrocytic differentiation, IGF1 to induce oligodendrocytic differentiation (panel E). After 6 days, cells were fixed and stained with anti-Map2 antibodies (panel A), anti-s100b antibodies (panel C) or anti-RIP antibodies (panel E). Merged images with DAPI staining are shown in panels B, D and F. Cells shown in panel C were rare populations which became positive for s100b in response to BMP2 and LIF.

FIG. 23 shows that tumorspheres formed by 005 tumor cells were positive for nestin and BLBP but negative for CD133. 005 tumor cells (panels A-C) and mouse normal stem cells (panels D-F) were stained with anti-nestin antibodies (panels A and D), anti-CD133 antibodies (panels B and E) and DAPI. Merged images are shown in panels C and F.

FIG. 24 shows lentiviral constructs tested in cell culture for production of pancreatic tumors. Kras G12D expression is tightly controlled by Doxycycline. The p53 siRNA cassette efficiently knocks down endogenous p53 expression.

FIG. 25 shows various virus combinations tested in cell culture. The pBob-1kBaM and 1KK2 viruses did not interfere with the expression of Kras, while they did efficiently inhibit/activate NFkB signal pathway (data not shown).

FIG. 26 shows injection in the pancreas parenchyma. The circled region is the best site for injection—the head of the pancreas, e.g., near the opening of the bile duct into the duodenum. Injection at this point produces an efficient infection after 2 microliters (titer of 10E9) CAG-GFP virus directly to the parenchyma.

FIG. 27 illustrates a single vector that activates Kras G12D and inactivates p53, Ink4/p16 and Smad4 in a cell type specific manner (Elastase promoter for acinar cells, carbonic anhydrase II promoter for ductal cells or nestin promoter for pancreatic progenitor cells).

FIG. 28 illustrates a vector that does not use the Cre/lox system but is also useful for activating Kras G12D and inactivating p53, Ink4/p16 and Smad4 in a cell type specific manner by using a tissue specific promoter, such as an Elastase promoter for acinar cells, a carbonic anhydrase II promoter for ductal cells, or a nestin promoter for pancreatic progenitor cells.

### DETAILED DESCRIPTION

The present invention relates to compositions, methods and systems for generating model tumors useful, e.g., for modeling treatments or screening putative therapeutics. The compositions include, but are not limited to, recombinant nucleic acids for expression of tumor associated phenotypes and viral packaging systems, e.g., lentiviral packaging systems, capable of transducing the nucleic acids into target cells transformable into tumors mimicking clinical tumors. Also included in the invention are recombinant animals with tumors that mimic those found in human patients. Such animals can be used as model systems for the study of tumor formation, prevention, treatment, and the like. The methods typically use viral vectors, such as lentiviral vectors to transduce tissues in vivo. See, e.g., reference 106. The amount of recombinant virus used and an appropriate choice of promoter, e.g., a tissue specific promoter, can be used to limit the cell types transduced to produce localized foci of transduced cells, thereby generating model tumor cells more representative of tumors in human patients.

A typical tumor model includes a tumor generated by transduction of a differentiated cell in vivo to produce neoplastic cells with characteristics highly correlated to those of tumor cells in common animal tumors, e.g., glioblastomas, pancreatic, or lung tumors. For example, a model tumor cell of the invention can be a cell in the tissue of a living animal, which has been transduced using a lentiviral vector particle of the invention to express oncogenes leading to neoplastic transformation of the cell. The lentiviral vector particles allow transformation of more representative adult differentiated cells in the more representative environment of the animal tissue, as compared to old art methods of generating tumor models.

In a typical method of generating a tumor model, a nucleic acid sequence of interest (e.g., an oncogene sequence or a tumor suppressor sequence) is incorporated into a transfer plasmid and packaged into a lentiviral particle along with other plasmids, such as, e.g., a transfer vector and a pseudotyping plasmid. The packaged particle is then introduced into a tissue of interest at a desired location where local cells can be transduced to express the sequence of interest. Alternately, the other plasmids are previously established in a packaging cell line. In many embodiments, the particles are directly introduced into the tissue using a fine syringe needle at a location known to be, or suspected of being, populated with cells that can transform into a cancer of interest. Alternately, particles are introduced into a variety of different tissue environments (e.g., at different tissue locations in an organ) and resultant tumors evaluated to identify cells with characteristics most similar to a cancer to be modeled. Optionally, the sequence of interest is selected, e.g., to encode oncogenes known to be, or suspected of being, associated with genesis of a clinical tumor of interest. Optionally, the vector system can be designed to interact with a desired target cell in such a way that the cell can be specifically transformed over other cells, identifiable as a cell transduced by the vector, and/or identifiable as a cell having a desired phenotype, as will be discussed below. The ability to transform cells at a specific location, transform specific cell types, identify types of transformed cells in a population of cells, and/or to transform with genes influencing the character of transformation, allows one to effectively select model cells that correlate highly to clinical tumors of interest.

### Methods of Generating Tumor Models

Tumor models generated according to the methods of the invention, e.g., in laboratory animals, can be quite similar in characteristics and responses to corresponding tumors in human patients. The present methods provide control and conditions more likely to produce model tumor cells and animals that are more similar to actual spontaneous human tumor cells and tumors than the more random prior art techniques. Control by an investigator over the selection of host animal, selection of a location in the animal to create a focus of transduction, selection of tumorigenic sequences for transduction, and selection of unique transformed cells from the site of transduction provides a certain ability to tailor model cells to closely mimic clinical tumors.

Generally, the methods of the invention include preparation of nucleic acid (e.g., plasmid) expression vectors, transfection of a packaging host cell to produce lentiviral transduction vector particles, injection of the particles to contact target cells of interest at a precise location in an organ or animal, growth of transformed cells, and identification of appropriate tumor model cells. Often, it is desirable to clone the identified tumor model cells for in vitro studies or seeding into tissues of a host animal for in vivo studies of the tumor.

### Vector Construction

The vectors of the methods, e.g., lentiviral vectors, typically include a transfer plasmid containing a coding sequence of interest, a plasmid encoding structural proteins and a reverse polymerase, and a plasmid encoding an envelope protein heterologous to the structural proteins. Such vector systems are previously described, e.g., in U.S. Pat. No. 6,013,516 to Verma; also, see reference 50. Alternately the necessary sequences and structural proteins can be present in host cells or packaging cells.

In preferred embodiments of the transfer plasmid, the sequence includes a constitutive promoter functionally associated to direct transcription and expression of the encoding sequence of interest, e.g., under defined conditions. It can be useful to include sequences encoding one or more detectable markers in the construct, e.g., signaling the presence of the construct in a transduced cell. For example, the transfer plasmid can include a sequence encoding a fluorescent protein detectable as a specific emission when the cell is interrogated with a specific excitation light wavelength. If the coding sequence of interest is located between the promoter and the marker sequence, the emission signal indicates both that the construct has been transduced into the target cell and that the promoter is likely promoting expression of the coding sequence of interest.

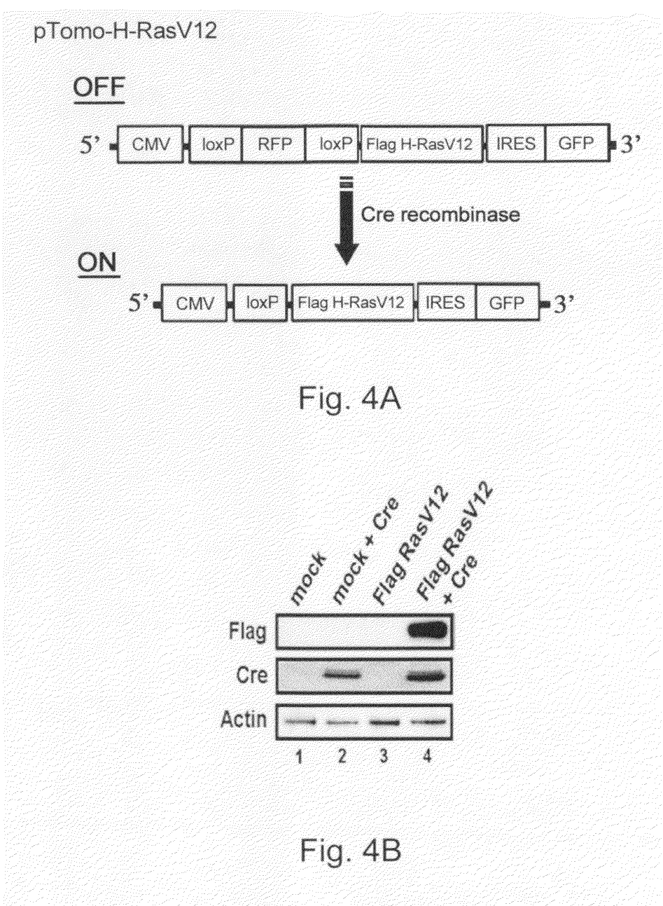

In another embodiment, the transfer plasmid encodes a detectable marker sequence immediately downstream from the promoter and upstream from a stop codon. Further, the marker sequence and stop codon can be bracketed by lox recombination sites. In the presence of Cre recombinase activity, the sequence between the lox cites is excised. Thus, if a transduced target cell does not include Cre activity, the marker will be expressed, but no sequences beyond the stop codon will be expressed. However, in the presence of Cre activity, the marker will not be expressed, but downstream sequences will be expressed. In a preferred embodiment, the transfer plasmid includes sequences in the order: promoter/lox/marker 1/stop/lox/sequence of interest/marker 2. When transduced into a cell without Cre activity, marker 1 will be expressed and detectable (as shown, e.g., in FIG. 4). When transduced into a cell with Cre activity, marker 1 and the stop will be excised so that the sequence of interest and marker 2 can be expressed. In a tissue containing both Cre positive and Cre negative cells, random transduction can result in marker 1 signal from the Cre negative cells and expression of the sequence of interest and a marker 2 signal in the Cre positive cells. If Cre positive cells are desirable target cells in the methods of generating tumor models, this scheme can selectively transform the desired target without transforming other cells at the location. What's more, the transduced cells could be readily identified as desired or undesired cells depending on whether they elicit either the signal of marker 1 or marker 2.

In other embodiments, selective transformation of desired target cells without transformation of nearby cells is accomplished without the Cre/lox system by using tissue specific promoters and injection of the viruses directly into the site at which a tumor is desired. These direct injections provide localized foci at desired locations, e.g., in the brain, pancreas, lung, or the like.

Construction of the recombinant nucleic acid constructs employed in the present invention can proceed according to genetic engineering techniques well known in the art. An introduction to such methods is found in available standard texts, including Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). Examples of techniques sufficient to direct persons of skill through in vitro amplification methods, useful in identifying, isolating and cloning nucleic acid diversity targets, including the polymerase chain reaction (PCR) the ligase chain reaction (LCR), Qβ-replicase amplification and other RNA polymerase mediated techniques (e.g., NASBA), are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem. 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

Host cells can be transduced with nucleic acids of interest, e.g., cloned into vectors, for production of nucleic acids and expression of encoded molecules, as described herein. In addition to Berger, Sambrook and Ausubel, a variety of references, including, e.g., Freshney (1994) Culture of Animal Cells, a Manual of Basic Technique, third edition, Wiley-Liss, New York and the references cited therein, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla. provide additional details on cell culture, cloning and expression of nucleic acids in cells.

### Production of Transducing Particles

RNA copies of transfer plasmids can be packaged into transducing particles by cotransforming all the necessary constituents into a packaging host cell. Packaging of such particles has been described, e.g., in U.S. Pat. No. 6,013,516 to Verma. For example, a lentivirus vector particle which infects non-dividing cells can be prepared by transfecting a suitable packaging host cell with appropriate nucleic acid vectors. A first vector nucleic acid (e.g., an expression or “packaging” plasmid) can encode a lentiviral gag and a lentiviral pol, wherein the gag and pol nucleic acid sequences are operably linked to a heterologous regulatory nucleic acid sequence. The first vector is typically defective for the nucleic acid sequence encoding functional ENV protein and devoid of lentiviral packaging sequences, e.g., upstream and downstream from a splice donor site to a gag initiation site of a lentiviral genome. A second pseudotyping vector can be provided with a nucleic acid sequence encoding a non-lentiviral ENV protein. A third transfer vector can be provided with a nucleic acid sequence containing a lentiviral packaging signal flanked by lentiviral cis-acting nucleic acid sequences for reverse transcription, packaging and integration. The transfer vector can include a heterologous nucleic acid sequence of interest, operably linked to a regulatory nucleic acid sequence and can include a less than full length gag structural gene (as a safety measure to prevent self-packaging). Cotransfection of the packaging host cell with the three vector plasmids can provide a system of nucleic acids functionally interacting to produce lentiviral transfer vector particles capable of transducing and expressing desired nucleic acid sequences into undifferentiated, or even differentiated non-dividing target cells.

The coding sequence of interest typically encodes a peptide sequence for expression intended to affect the phenotype of the target cells in order to force or increase the likelihood the target cells will be transformed to a tumor or to become malignant. Some extent of control over the tumor character can be exercised by the choice of coding sequences of interest. This choice can be influenced by, e.g., the characteristics of the target cell, cancer to be modeled, and oncogenes and/or suppressors present in the tumor to be modeled. In many cases, it is desirable that the vector include two or more coding sequences of interest. Typical coding sequences of interest useful in the methods of generating model tumors include, e.g., ras, myc, src, AKT, Aurora-A kinase, BAFF, Kras, a sequence coding for an agent that represses expression or activity of a tumor suppressor, a sequence encoding a serine kinase, a sequence encoding a G-protein, a sequence encoding a threonine kinase, a sequence encoding a tyrosine kinase and/or the like. For example, tumor suppressors of interest in pancreatic cancer include p53, Ink4/p16, and Smad4.

The methods of the invention can include combination of the three nucleic acid vectors in order to produce a recombinant virion or recombinant retrovirus construct. As discussed above, a first vector provides a nucleic acid encoding a viral gag and a viral pol (alternately, these can be separately encoded on separate plasmids). These sequences can encode, e.g., reverse transcriptase, integrase and protease-enzymes necessary for maturation and reverse transcription. Preferably, the viral gag and pol are derived from a lentivirus, e.g., a murine retrovirus or possibly HIV. Most preferably, the nucleic acids of the invention use one or more of the vectors shown in FIGS. 15A to 15F, or use these plasmids as starting material to prepare the vectors.

A second vector can provide a nucleic acid encoding a viral envelope (env). The env gene can be derived from any virus, including retroviruses. The env can be an amphotropic envelope protein which allows transduction of cells of human and other species, or may be ecotropic envelope protein, which is able to transduce, e.g., only mouse and rat cells. Further, it can be desirable to make the recombinant virus specific for a particular target cell by linkage of the envelope protein with a cell-specific antibody or a particular ligand. Retroviral vectors can be made target specific by inserting, for example, a glycolipid, or a protein. Targeting is often accomplished by using an antibody to target the retroviral vector. Those of skill in the art will know of, or can readily ascertain without undue experimentation, specific methods to achieve delivery of a retroviral vector to a specific target.

Examples of retroviral-derived env genes include, but are not limited to: Moloney murine leukemia virus (MoMuLV), Harvey murine sarcoma virus (HaMuSV), murine mammary tumor virus (MuMTV), gibbon ape leukemia virus (GaLV), human immunodeficiency virus (HIV) and Rous Sarcoma Virus (RSV). Other env genes such as Vesicular stomatitis virus (VSV) (Protein G) can also be used.

The vector providing the viral env nucleic acid sequence is typically operably associated with a regulatory sequence, e.g., a promoter or enhancer. Preferably, the regulatory sequence is a viral promoter functional in eukaryotic systems. The regulatory sequence can be any eukaryotic promoter or enhancer, including for example, the Moloney murine leukemia virus promoter-enhancer element, the human cytomegalovirus (CMV) promoter or the vaccinia P7.5 promoter. In some cases, such as the Moloney murine leukemia virus promoter-enhancer element, these promoter-enhancer elements are located within or adjacent to the LTR sequences.

The third vector nucleic acid provides the cis-acting viral sequences that provide for packaging, integration, etc. (the transfer vector). Such sequences can include the psi packaging sequence, reverse transcription signals, integration signals, viral promoter, enhancer, and polyadenylation sequences. The third vector also contains a cloning site for a heterologous nucleic acid sequence of interest to be transferred to a non-dividing cell. Schematic illustration of nucleic acid vectors suitable for recombination and transfection of packaging host cells for production of vector particles are provided in FIGS. 15A to 15F.

The method of producing the recombinant retrovirus of the invention is different from most standard helper virus/packaging cell line methods old in the art. The three or more individual vectors used to co-transfect a suitable packaging cell line optionally collectively contain all of the required genes for production of a recombinant virus for infection and transfer of nucleic acid to a non-dividing cell. Consequently, there is typically no need for a helper virus to prepare the vector particles, though one can be used in alternate embodiments.

### Injecting Lentiviral Vector Particles to Produce Local Foci of Model Cells

Many human cancers initiate as a singly transformed cell at a locus and can progress to an enlarged tumor and possible metastatic dispersion. The present methods are able to reproduce this pattern in a way that mimics natural processes better than any tumor model presently available. The vector particles, e.g., lentiviral vector particles, can be injected into specific localized cites to produce foci of transformed cells. The foci produced mimic characteristics of spontaneous tumors at early clinical stages of natural progression, e.g., as normally found in human patients with an early cancer disease state. These vector particles optionally use a Cre/lox system as described above to target specific cells or they rely on tissue specific promoters and direct injection of the viral particles to produce localized tumor models.

The vector particles can be injected to controllably contact a specific organ, tissue, or cell type. It is an aspect of the invention that desired target cells can be transformed without random or generalized transformation of other cells. Because the vector particles can transduce differentiated cells, the virus does not have to disperse far before binding a target cell. This also allows productive injection of small volumes, further reducing the size and dispersion of transductions and target cell transformations. Injection, herein, can refer, e.g., to injection through a conduit (e.g., hollow needle) or insertion of particles from an intrusive appliance, such as, e.g., needle surface or a needle tip.

In many embodiments, model cells can be specifically generated at a particular location in an organ or tissue of interest, e.g., in an appropriate laboratory animal. For example, very fine needles can be directed to minute locations within an organ to regions known to be, or suspected of being, populated with desired target cells. Different regions of various organs have long been known in the art to have different proportions of various cell types. In methods of the invention, a technician, or robotically actuated device, can insert the tip of a needle to a location containing desired cells for injection of a number of vector particles. In this way, desired cells (e.g., cells originating from a desired germ layer, cells with a desired phenotype, cells most competent for the desired transformation, etc.) can be specifically targeted geographically. In certain embodiments of the invention, vector particles are injected to contact a specific portion of an organ, specific tissue of an organ, a specific cell type of a tissue, or to contact a specific cell. For example, to induce a pancreatic cancer tumor, e.g., a pancreatic intraepithelial neoplasia (PanIN), injection into the parenchyma is preferred.

In preferred embodiments of the methods, vector particles are injected to a precise location in a low controlled volume. For example, particles can be injected to a desired location in an animal within a precision of 10 mm, 1 mm, 0.1 mm, 10 μm, or 1 μm, or better. In methods of injecting the vector particles, the injection volume can be about 1 ml, 0.1 ml, 10 μl, 1 μl, 0.1 μl, or less. In order to further reduce dispersion, it can be beneficial to complete an injection over the course of about 10 minutes, 1 minute, 20 seconds, 10 seconds or about 5 seconds. Dispersion of transducing particles can further be physically reduced by providing the vector particles in a high titer. For example, the vector particles can be injected at a concentration of about 10⁴, 10⁵, 10⁶, 10⁷, 10⁸, 10⁹, 10¹⁰, 10¹¹, 10¹², or more functional particles per milliliter, thus allowing the particles to transduce cells in a smaller tissue foci. In a preferred embodiment, transducing vector particles are injected to contact tissue containing target cells at a location with a volume less than 1 cubic centimeter (cc), less than 0.1 cc, less than 0.01 cc, less than 1 cubic millimeter (mm³), less than 0.1 mm³, less than 0.1 mm³, less than 0.01 mm³, than 0.001 mm³, than 0.001 mm³, or less. Optionally, vector particles of the invention can contact target cells in liquid suspensions, e.g., in body fluids or in vitro culture media. Optionally, a given target cell host animal experiences only a single injection.

In many cases, there are advantages to using genetically well-characterized laboratory animals as the subject of vector particle injections in the methods of generating model tumor cells. For example, transgenic animals can be useful platforms for the cancer models of the invention, e.g., for studying oncogene and tumor suppressor function and testing modulators thereof.

In one aspect, the invention includes knock out and/or transgenic target host cell animals. For example, non-human laboratory animals that comprise a knock out in an endogenous oncogene or tumor suppressor gene can be made and can additionally include a heterologous oncogene or tumor suppressor gene (e.g., from a human source) corresponding to the knock out. This type of knock out animal, which can be, e.g., a mouse with a human oncogene or tumor suppressor can help more accurately mimic the underlying biology for the clinical tumors to be modeled.

Transgenic animals can also include those that endogenously express, e.g., CRE recombinase, e.g., in one or more specific cell or tissue types of interest (e.g., through use of a heterologous promoter linked to a CRE coding sequence, or by homologous recombination into a promoter region that displays tissue specific expression).

A transgenic animal is typically an animal that has had DNA introduced artificially into one or more of its cells. This is most commonly done in one of two ways. First, DNA can be integrated randomly by injecting it into the pronucleus of a fertilized ovum. In this case, the DNA can integrate anywhere in the genome. With this approach, there is no need for homology between the injected DNA and the host genome. Second, targeted insertion can be accomplished by introducing heterologous DNA into embryonic stem (ES) cells and selecting for cells in which the heterologous DNA has undergone homologous recombination with homologous sequences of the cellular genome. Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (i.e., random insertion).

One common use of targeted DNA insertion is to make knock-out mice. Mice provide a very useful laboratory animal, due to the ease with which the animals can be bred, made recombinant, etc. Typically, when making knock outs in mice (or other laboratory animals), homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

In preferred embodiments of the methods, substantially all the transductions or all target cell transductions take place at the location of the vector particle injection. For example, it is preferred that all the resultant cell transformations to tumor cells take place in a contiguous region, e.g., within the preferred volumes of injected contact, discussed above. In methods of the invention, tumors are induced only within a single organ, only within a contiguous tissue of an organ, or within a group of cells sharing the same arteriole blood supply or venule drainage. It is preferred that tumors are not induced in two or more different locations within the body of the injected animal. It is preferred that tumors are not induced at a location separated significantly from the location of the vector particle injection.

### Identifying Model Tumor Cells

After target cells have been transduced, e.g., into a the brain or pancreas of a mouse, desired model tumor cells can be identified. Identification can be based on one or more characteristics of interest in the transduced cells. In many cases, the characteristics of interest are visible characteristics, such as signals form a detectable marker, or cell morphology. In other cases, the characteristics can require analyses, e.g., for genetic changes or enzymatic activity.

In many cases, the vector systems of the methods include marker sequences that allow identification of transduced cells by the presence of a transduction marker signal (e.g., a fluorescent emission). Further, the vector systems often include marker sequences arranged to only provide a signal if an associated sequence of interest (e.g., an oncogene) has been expressed in the cell.

Typically, model tumor cells or model tumors in recombinant animal models can be identified by the same detection methods, or combination of methods, known in the art for identification of the tumor intended to be modeled. For example, model tumor cells can be identified and characterized by parameters, such as, e.g., increased density of a cell type at the injection location, necrosis of contiguous cells, overall cell morphology and staining characteristics, invasion of surrounding tissue by the cells (metastasis), nuclear pleomorphism (many characteristic polymorphisms are known and detectable with specific staining and microscopy), degree of differentiation, giant cell formation (e.g., syncytia), increased mitotic cells (e.g., often as a result of uncontrolled growth), lack of contact inhibition, and increased nucleus to cytoplasm ratio, and/or the like. In some cases, specific staining techniques can employ particular stains, detectable antibodies or chromogenic enzyme substrates to help characterize and identify a tumor. In many cases, the cell karyotype or expression of certain genes or can help identify a tumor. In most cases, identification of a clinical tumor is not simply the matter of a single observation, but a reasonably confident identification based on evaluation of multiple tumor characteristic parameters. Therefore, it is reasonable and preferred that model tumor cells of the invention be identified and selected subject to the same type of standard evaluation.

Transduced cells remaining in situ at the location where target cells were contacted and transformed can be model tumor cells useful as tools to investigate potential therapeutics and treatment methods. This can be considered one type of animal model of the invention. A unique advantage of this technique is that it provides a course of disease that can closely mimic the clinical course of the related disease state in a human or other animal. This technique allows study of treatments for a tumor at a desired location and at any stage of growth. For example, this technique allows investigation of single tumors in a single body along any point in the time line from undetectable tumor foci, to initial growth, to palpable tumor, through metastatic phases. Thus, investigation can proceed without interference from the effects found in old art models, such as problematic interactions between multiple tumors, disseminated tumors, tumors of unknown location, tumors in the wrong organs, and the like.

Alternately, the model cells can be harvested, e.g., using known methods of primary cell culture and cloning, for use in methods of in vitro tumor studies. In vitro studies can include, e.g., screening of appropriate anticancer therapeutics, testing therapeutic combinations, etc.

Seeding Model Cells into Animal Tissues

Cultured and/or cloned model cells of the invention can be used in methods of creating animal models of tumorigenesis, pharmacology and cancer treatment. Model cells, generated as described above, can be implanted into a host animal at an appropriate location to create an animal tumor model that can be highly representative of the clinical tumors of interest.

Compatibility between the host animal and the tumor model cells can facilitate transplant efficiency and reduce tumor study variables associated with immune rejection phenomenon. In one embodiment of methods for preparing animal models of tumors, model cells of a particular type of clinical tumor are generated as described above, then they are seeded to appropriate locations in a host animal. In preferred embodiments, the model tumor cells are seeded into the same strain of animal as the target cell host animal from which the model tumors were transformed. In preferred embodiments, the model tumor cells are seeded into the same organ or tissue as the target cells from which the model cells were derived. Optionally, the host animal is a species different from the model source or the cells are seeded to a different organ. Optionally, the tumor model animal host is immunologically incompetent in such a way as not to reject the implanted model cells.

In a typical embodiment, model cells are cloned and cultured before being seeded into a host animal. For example, the model cells can be identified and separated from surrounding tissues according to well-established primary culture techniques. The model cells could have been engineered to have a phenotype allowing them to grow in a media toxic or otherwise unsuitable for other cells of the harvested tissue. Cloning of the model cells, e.g., by dilution of cell sorter, can help ensure a pure and well defined seeding material. An adequate number of model cells can be grown, e.g., in suspension or surface culture, then suspended in a volume of appropriate fluid for seeding into the host animal by injection. As with the transduction methods discussed above, the location of the injection can be precisely controlled, as required for a particular animal system and associated studies.

The present methods have demonstrated an ability to efficiently establish a tumor using a relatively small number of seeded cells. This provides many advantages, including those associated with precisely located small early phase tumor foci, as discussed above. In preferred embodiments, the host animal is seeded with less than 10,000, less than 1000, less than about 100, less than about 50, about 10, or less model cells, to establish a viable tumor foci in the host animal.

### Methods of Screening for Modulators of Tumorigenesis

Modulators of cell transformation, progressive tumor growth and tumor cell metastasis can be screened using the model cell and model animal systems generated using the methods of the invention. For example, researchers can model cells or animals with small molecule compounds or biomolecules and evaluate the modulatory effects on tumorigenesis in the model system, e.g., pancreatic intraepithelial neoplasia or glioblastoma multiforme tumor models. Putative modulators that appear to provide significant modulation of tumorigenesis in the model systems can be selected for preclinical clinical and clinical studies for safety and efficacy in treatment of the clinical tumor being modeled by the systems of the invention. The modulators are also optionally tested for efficacy and safety, etc, in animal models as provided herein.

Desirably, a modulator compound is, e.g., an inhibitor of the oncogene or a potentiator of the tumor suppressor.

Essentially any available compound library can be screened in using the tumor models of the invention and activity of the library members in modulating tumor characteristics can be assessed, optionally in a high-throughput fashion.

Many libraries of compounds are commercially available for tumor modulation in the screening methods of the invention, e.g., from the Sigma Chemical Company (Saint Louis, Mo.), Aldrich chemical company (St. Louis Mo.), and many can be custom synthesized by a wide range of biotech and chemical companies. Current compound library providers include Actimol (Newark Del.), providing e.g., the Actiprobe 10 and Actiprobe 25 libraries of 10,000 and 25,000 compounds, respectively; BioMol (Philadelphia, Pa.), providing a variety of libraries, including natural compound libraries and the Screen-Well™ Ion Channel ligand library which are usefully screened against oncogenic receptors, as well as several other application specific libraries; Enamine (Kiev, Ukranie) which produces custom libraries of billions of compounds from thousands of different building blocks, TimTec (Newark Deleware), which produces general screening stock compound libraries containing >100,000 compounds, as well as template-based libraries with common heterocyclic lattices, libraries for targeted mechanism based selections, including kinase modulators, GPCR Ligands, channel modulators, etc., privileged structure libraries that include compounds containing chemical motifs that are more frequently associated with higher biological activity than other structures, diversity libraries that include compounds pre-selected from available stocks of compounds with maximum chemical diversity, plant extract libraries, natural products and natural product-derived libraries, etc; AnalytiCon Discovery (Germany) including NatDiverse (natural product analogue screening compounds) and MEGAbolite (natural product screening compounds); Chembridge (San Diego, Calif.) including a wide array of targeted or general and custom or stock libraries; ChemDiv (San Diego, Calif.) providing a variety of compound diversity libraries including CombiLab and the International Diversity Collection; Comgenix (Hungary) including ActiVerse™ libraries; MicroSource (Gaylordsville, CT) including natural libraries, agro libraries, the NINDS custom library, the genesis plus library and others; Polyphor (Switzerland) including privileged core structures as well as novel scaffolds; Prestwick Chemical (Washington D.C.), including the Prestwick chemical collection and others that are pre-screened for biotolerance; Tripos (St. Louis, Mo.), including large lead screening libraries; and many others. Academic institutions such as the Zelinsky Institute of Organic Chemistry (Russian Federation) also provide libraries of considerable structural diversity that can be screened in the methods of the invention. Pyrazole libraries are one useful library for screening in the present invention (see, Mol. Pharmacol. 2006 April; 69(4):1413-20).

In one aspect, the present inventions include methods of screening for compounds that modulate tumorigenesis or cancer progression. A model cell, or animal comprising a tumor model of the invention, can be contacted with a test compound and a resulting effect of the test compound on the model cells or animal can be monitored. Compounds that display modulation of one or more model cell phenotype (e.g., tumor cell or model animal characteristic parameters: tumorigenicity, development of cancer markers, cellular density, necrosis, tumor invasion into the surrounding normal tissues, prominent nuclear pleomorphism, giant cell formation, lack of contact inhibition, increased nucleus to cytoplasm ratio and increased mitotic figures, etc.) can be considered modulators of the oncogene or tumor suppressor. Compounds identified by these methods are also a feature of the invention.

Putative modulators can contact model systems to determine levels of modulation at every stage of tumorigenesis. Modulators can contact, e.g., target cells in the presence of vector particles, model cells selected as correlated to clinical tumor cells, and/or model animals with model cells at loci in one or more organ or tissue.

For example, animals or target cells shown to generate model tumor cells consistently in response to transduction with lentiviral vector particles of the methods above can be contacted with putative modulators to see the effect they have on transformation of target cells. Modulators that reduce the transformations may be, e.g., putative prophylactic pharmaceuticals against carcinogenesis.

In another example, model cells in vitro can contact putative modulators for evaluation of changes in tumor associated characteristics (parameters). Putative modulators that appear to influence one or more parameters of interest can be selected for further study, e.g., as pharmaceuticals or foundations for molecular engineering of related more potent modulators.

In another example, putative modulators can be administered to animal models of the invention for evaluation of modulatory effects. Administration can be, e.g., oral, intravenous or direct to the model tumor. The animal can be evaluated for modulation of clinical signs associated with a particular cancer, such as the presence of certain antigens, tumor associated enzymes, tumor growth, tumor invasiveness, general animal health, and the like. The imbedded model cells can be evaluated by invasive (e.g., biopsy, catheter, endoscopic techniques) or not-invasive techniques (e.g., x-ray, CAT scan, NMR) to monitor parameters of the model tumor in response to the administration.

### Administering Modulators/Dosages of Modulators

Putative modulators, having been found to be effective modulators, can be administered clinically in the treatment and prevention of cancer. For example, a test molecule found to statistically affect a tumor associated parameter (e.g., as compared to an appropriate control), is optionally evaluated in preclinical and clinical studies to determine effectiveness as a drug for reducing morbidity or mortality from an associated cancer (e.g., the cancer being modeled by the model system of the invention). In many cases, a modulator found effective in modulating model cells is next administered to a model animal with the same model cells or to an animal with the associated naturally occurring cancer. The animal model, e.g., one generated using the methods provided herein, can be useful in determining the proper route of administration, an appropriate dosage form or dose regimen, etc. The animal model is also optionally used to test modulators for effects on tumor invasiveness. Any techniques described herein or known to those of skill in the art to be useful with respect to model cells or animals are optionally used with the model animals provided herein, e.g., for testing drugs and/or other treatments.

The modulators of the invention are administered in any suitable manner, optionally with one or more pharmaceutically acceptable carriers. Suitable methods of administering such nucleic acids in the context of the present invention to a patient or model animal as provided herein are available, and, although more than one route can be used to administer a particular composition, a particular route can often provide a more immediate and more effective action or reaction than another route. The efficacy of these routes is optionally tested using model animals generated with the techniques provided herein.

Pharmaceutically acceptable carriers are determined in part by the particular composition being administered, as well as by the particular method used to administer the composition. Accordingly, there are a wide variety of suitable formulations of pharmaceutical compositions of the present invention. Compositions are optionally administered by a number of routes including, but not limited to: oral, intravenous, intraperitoneal, intramuscular, transdermal, subcutaneous, topical, sublingual, spinal, or rectal administration. Compositions can be administered via liposomes (e.g., topically), or via topical delivery of naked DNA or viral vectors. Such administration routes and appropriate formulations are generally known to those of skill in the art and can be tested using model animals of the present invention.

The compositions, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged modulators can be presented in unit-dose or multi-dose sealed containers, such as ampules and vials.

The dose administered to a lab animal, e.g., one with a tumor generated by the methods provided herein, or patient, in the context of the present invention, is sufficient to affect a beneficial therapeutic response in the patient over time. The dose is determined by the efficacy of the particular vector, or other formulation, and the activity, stability or serum half-life of the polypeptide which is expressed, and the condition of the patient, as well as the body weight or surface area of the patient to be treated. The size of the dose is also determined by the existence, nature, and extent of any adverse side-effects that accompany the administration of a particular vector, formulation, or the like in a particular patient. In determining the effective amount of the vector or formulation to be administered in the treatment of disease, the physician evaluates local expression in the tissue or cell of interest, or circulating plasma levels, formulation toxicities, progression of the relevant disease, and/or where relevant, the production of antibodies to proteins encoded by the polynucleotides. These parameters are also optionally optimized using model animals of the invention. The dose administered, e.g., to a lab animal or patient are typically in the range equivalent to dosages of currently-used therapeutic proteins, adjusted for the altered activity or serum half-life of the relevant composition. The vectors of this invention can supplement treatment conditions by any known conventional therapy (e.g., diet restriction, etc.).

For administration, formulations of the present invention can be administered at a rate determined by the LD-50 of the relevant formulation, and/or observation of any side-effects of the vectors of the invention at various concentrations, e.g., as applied to the mass or topical delivery area and overall health of the patient. Administration can be accomplished via single or divided doses and tested, e.g., using animals with tumor models generated as provided herein.

In another aspect of the methods of the invention, the vector particles, including therapeutic coding sequences of interest can be specifically injected into clinical tumor tissue, e.g., to terminate the transformation of tumors or neoplastic cells. For example, the vectors, encoding, e.g., tumor suppressors, differentiating genes, RNA, antisense DNA, or the like, can be precisely injected to locally convert tumor cells back to normal differentiated cells.

### Vectors

Vectors of the invention include recombinant nucleic acids useful in transformation of target cells to provide model tumors that correlate well to clinical tumors, e.g., glioblastomas, pancreatic, or lung tumors. Vector particles include vector nucleic acids packaged, e.g., in a lentiviral capsid.

One recombinant nucleic acid composition of the invention has a sequence with a promoter upstream from a first lox recombination site and a second lox recombination site bracketing a nucleic acid sequence encoding a stuffer sequence (such as an optional marker encoding sequence). Downstream from the lox sites is one or more coding sequences of interest and nucleic acid sequence encoding a detectable first marker. In a preferred embodiment, the nucleic acid includes sequences in the order: promoter, first lox, stuffer, second lox, coding sequence of interest, and first marker sequence. It is preferred that the promoter be a constitutive promoter, such as a CMV promoter, and the marker be a fluorescent protein. To facilitate packaging into a lentiviral vector particle, the nucleic acid includes a lentiviral packaging sequence site, such as a psi site.

In many embodiments, the coding sequence of interest can have an effect on a tumor associated characteristic of the target cell. The choice of one, two, or more coding sequences of interest can induce transformation of the target cell and influence the character of the transformed target cell. The choice of target cell location, target cell, and sequences of interest can combine to control the type of model cells produced. Exemplary target cells can include, e.g., a neuronal cell, a pancreatic cell, a prostate cell, a breast cell, a liver cell, a skin cell, a differentiated cell, a mature astrocyte, a lung cell or the like. Exemplary sequences of interest include, e.g., ras, myc, src, AKT, Aurora-A kinase, BAFF, Kras, a sequence coding for an agent that represses expression or activity of a tumor suppressor, a sequence encoding a serine kinase, a sequence encoding a G-protein, a sequence encoding a threonine kinase, a sequence encoding a tyrosine kinase, a signal transduction protein, an RNAi that inhibits expression of a tumor suppressor, an siRNA that inhibits expression of a tumor suppressor, and/or the like. Tumor suppressors of interest include, but are not limited to, p53, Ink4/p16, Smad4, and the like. For example, a single vector comprising a coding sequence for Kras and sequences to inhibit multiple tumor suppressors, such as p53, Ink4/p16, and Snad4 is optionally used to transduce a pancreatic cell.

The recombinant nucleic acid can be present in a competent packaging host cell along with a lentiviral packaging plasmid that encodes a lentiviral gag protein. The packaging cell can also include a pseudotyping plasmid that encodes an envelope protein heterologous to the gag protein. The packaging cell can process the recombinant nucleic acid into a lentiviral particle capable of transducing the nucleic acid into a target cell of interest.

### Tumor Models

Tumor cell models and animal models comprising the model cells generated by the methods of the invention are additional aspects of the invention. Cells transformed according to the methods of the invention described above can provide models for investigation of corresponding human and animal clinical tumors and malignancies. The cells can be cloned and cultured to provide in vitro models, e.g., for discovery therapeutic agents by mass screening. The cells can be transduced directly into a laboratory animal, or seeded into a laboratory animal for in vivo testing of therapeutics or medical devices.

### Tumor Induction Models

An aspect of the invention is the transduced target cells at a localized focus in a tissue of a host target cell animal after contact with the lentiviral vector particles of the invention. Experience with the system can show consistent local transformation of identifiable transduced target cells. These transduced cells are useful, e.g., in screening for modulators of transformation, as described above in the methods section.

In one embodiment, the transduced target cell is a cell in a host animal at a location of interest to an investigator. The cell can be positive or negative for Cre recombinase. The cell can be from any desired organ or tissue, but preferably the cell is the only transduced cell in the animal, or a member of a substantially contiguous group of similarly transduced cells. The transduced cell can be, e.g., a neuronal cell, a pancreatic cell, a prostate cell, a breast cell, a liver cell, a skin cell, a differentiated cell, a mature astrocyte, a lung cell, or the like. The transduced cell can be derived from embryonic ectoderm, mesoderm or endoderm.

### Model Tumor Cells

Compositions of the invention include model tumor cells generated by the methods of the invention and, e.g., shown to include tumor associated parameters similar to the clinical tumors they are intended to model, e.g., GBM or PanIn. Preferred model tumor cells respond to modulating moieties in the same way as the associated clinical tumors. These cells can be useful in research for cancer cures and methods of cancer prevention.

Typical model cells of the invention will comprise a recombinant nucleic acid, as described above, having a sequence with a promoter upstream from a first lox recombination site and a second lox recombination site bracketing a nucleic acid sequence encoding a stuffer sequence, a promoter sequence upstream from the lox recombination sites, a coding sequence of interest, and nucleic acid sequence encoding a first marker. Preferably, both the coding sequence of interest and the first marker are down stream from the lox recombination sites. Preferably, the sequence bracketed by the lox sites include a stop codon in functional register. Preferably the nucleic acid includes a lentiviral packaging site. In model cells expressing Cre recombinase, the stuffer sequence will no longer exist in the recombinant nucleic acid.

Model cells, as described, typically express characteristics of the tumors being modeled and are readily identified by a marker signal. In preferred embodiments, the model cells, e.g., cells that are susceptible to tumor transformation, are transformed into tumors or actively malignant tumor cells. Some cells, e.g., certain types of stem cells, are more susceptible than others to tumor transformation, e.g., because of a differentiated state. In making tumor models in animals, areas that have these types of cells are preferred injection sites. The model cells can be in situ at the location where parental target cells were originally transduced. The model cells can be malignantly dispersed to other locations within the target cell host animal. The model cells can be cloned into cell lines capable of culture in laboratory cell media. The model cells can be present at one or more locations (e.g., seeded) in a tumor model animal different from the original target cell animal.

### In Vivo Models

In vivo models of the invention can provide representative tumors in living animal tissues, thus replicating the environment and challenges facing investigators of tumor modulators and therapy courses.

The in vivo models can include a non-human animal, e.g., a laboratory research animal, comprising the recombinant nucleic acids discussed above. The model animals can comprise the model cells described above. For example, a model animal can comprise a recombinant Cre recombinase, expressed in one or more cell or tissue of the animal. The animal models can comprise a recombinant nucleic acid that encodes the sequence of a first lox recombination site and a second lox recombination site bracketing a nucleic acid sequence encoding a stuffer sequence, or a recombinant nucleic acid that encodes a sequence comprising recombined lox recombination sites. The model animal can further include a promoter sequence upstream from the recombined lox recombination sites. The animal can include a coding sequence of interest and a first marker nucleic acid sequence encoding a first marker, each downstream from the recombined lox recombination sites. In many embodiments, the recombinant nucleic acid will include a lentiviral packaging site (psi) remnant from systems used to transduce the recombinant nucleic acid into the animal cells.

In other embodiments, the recombinant nucleic acid does not contain a lox recombination site and the animal does not necessarily comprise Cre recombinase. These alternative animal models are directly injected with viral vector particles that include coding sequences, e.g., for oncogenes such as Kras, under the control of a tissue specific promoter. For example, carbonic anhydrase II, elastase I, or the like is optionally used to promote expression in a particular type of tissue to produce a localized tumor. Other tissue specific promoters include, but are not limited to, CAII for ductal cell in pancreatic tissue or an alveolar cell in lung tissue, E1a1 for acinar cells in pancreatic tissue, and the like.

The animal can be any suitable to provide cells transducible or transformable to mimic a clinical tumor if interest, provide an environment for growth of transduced or transformed cells into model cells of the invention, and/or provide an location for model cells similar to locations typical of clinical tumors. Preferred animals are well-characterized animals, such as laboratory animals. For example, the model animals can be derived from mice, rats, rabbits, monkeys, and the like. The site of model cells in the animals can be any suitable organ or tissue, as described above. The coding sequence of interest in the recombinant nucleic acid can preferably be sequences that influence the tumor specific characteristics of the model cells, such as oncogenes, oncogene suppressors or suppressors of oncogene suppressors, as discussed above.

### EXAMPLES

The following examples are offered to illustrate, but not to limit the claimed invention.

The present invention provides a new way to induce oncogenic events in adult mice in a cell type specific manner using lentiviral vectors. The vectors are able to infect non-dividing cells in most organs of adult mice. Cre inducible vectors were generated in a CMV-loxP-RFP-loxP-interest gene-IRES-GFP construct. We chose Cre-loxP systems to establish cell type specific inducible oncogenes. Because this vector uses red fluorescent protein (RFP) as a stuffer, it is extremely easy to examine the Cre activity in vitro and in vivo. The vector also includes green fluorescent protein (GFP) so cells can be detected after exercising the RFP and gene of interest expression. Alternatively, a non-Cre system with a tissue specific promoter is used for direct injection of the viral packaging particle into a desired tissue. Examples include, but are not limited to, CAII promoter for direct injection into ductal cell pancreatic tissue or alveolar cells in lung tissue, Ela1 for acinar cells in pancreatic tissue. Others choices will be well known to those of skill in the art.

Injection of the vector construct into adult mice having the cell type specific Cre recombinase establishes a new way to induce oncogenic events in a cell type specific manner in adult mice. This technique provides information on the origin of tumors in the cells. As few as 40 to 50 cells in the brain transduced with a lentivector comprising activated H Ras and Akt can make lead glioblastomas in 60 to 90 days. The resulting tumor cells can show histological character, and other tumor-associated characteristics, very similar to those of human glioblastomas. Moreover, the tumor cells are able to survive in culture and form neurosphere-like structures. Further, As few as 100 cells, or less, can regenerate a robust glioma; these tumor cells can therefore be tumor stem/initiating cells that can provide relevant therapy target models. Thus, the vectors can be useful tumor models, e.g., for drug screening. Similar technology can be applied to any animals and any organs, e.g., pancreas, breast, prostate, and the like.

In the present studies, model cells were locally transduced in specific regions of a mouse brain. FIG. 16 schematically shows cells in adult dentate gyrus and potential mechanism of the formation of GBMs. FIG. 16A is a schematic diagram of cells in the dentate gyrus: neural stem cells located in the subgranular zone of the dentate gyrus are GFAP+/nestin+. Mature astrocytes which predominantly exist in the hilus are GFAP+/nestin−. GZ: granular zone, SGZ: subgranular zone, HL: hilus. FIG. 16B shows a schematic diagram of potential steps during the tumor formation of GBMs. Combined activation of H-Ras and AKT with loss of p53 in GFAP+/nestin− cells causes the generation of GFAP−/nestin+ brain tumor stem/initiating cells. Oncogenic stimuli in the nestin positive cells did not frequently cause the tumor formation, indicating that GFAP+/nestin− mature astrocytes around the dentate gyrus are most likely to be the cell of origin of the tumor. Unknown factors in the hippocampus area may play role(s) in this transformation of the cells, because GFAP+/nestin-mature astrocytes in the cerebral cortex were rarely transformed by the same oncogenic stimuli. Normal cells such as neurons, oligodendrocytes and astrocytes may be trapped by the tumor during its development resulting in the formation of tumor containing multilineage cells.

### Example 1

### Generation of Cre-Inducible Lentiviral Vectors

Brain tumors including GBMs in adults are thought to arise from a series of somatic mutations leading to the activation of oncogene(s) or inactivation of tumor suppressor(s) that occurs in a few or even single founder cells [48][49]. To recapitulate this initiation and progression of the GBMs, it can be important to induce these oncogenic event(s) leading to the formation of glioblastomas in a small population of a specific type of cells in an adult mouse brain. To achieve this, we utilized lentiviral vectors that can transduce both the dividing (such as neural stem cells and neural progenitor cells) and the rarely-dividing cells (such as terminally differentiated astrocytes) [50]. To target specific cell types, we chose Cre-loxP system which is thought to be a very reliable method to express or delete the target gene specifically in the Cre expressing cells [51]. We have generated Cre-inducible lentiviral vectors capable of inducing the expression of oncogene(s) in an adult mouse brain in a cell type specific manner.

Since Ras pathway has been shown to be involved in the formation of gliomas [52][53], we first chose to express oncogenic p21-Ha-RasV12 (H-RasV12) in a cell type specific manner in an adult mouse brain. We constructed Cre-inducible H-RasV12 lentiviral vector, pTomo-H-RasV12, which contains a human cytomegalovirus immediate-early promoter (CMV)-loxP-red fluorescent protein (RFP)-loxP-Flag-H-RasV12 region followed by IRES-GFP (internal ribosomal entry site-green fluorescent protein; FIG. 4A). RFP is used as a “stuffer” sequence between the CMV promoter and H-RasV12. Flag tag was added in the N-terminal portion of H-RasV12, which is known to have no effect on Ras function [54]. The stuffer was flanked by a pair of loxP sites, allowing its excision by Cre recombinase followed by expression of H-RasV12. The “stuffer” sequence produces RFP protein and the cells infected with this vector also express GFP. Theoretically, this vector can express GFP and RFP in the absence of Cre, whereas it can express GFP and H-RasV12 in the presence of Cre. Thus, this vector is able to express Flag H-RasV12 specifically in the Cre expressing cells, and those cells can be detected as the cells expressing GFP but not RFP.

As expected, immunocytochemical analysis showed that the expression of RFP was dramatically reduced in the presence of Cre recombinase (data not shown), indicating that the “stuffer” RFP had been successfully deleted by Cre recombinase. Furthermore, Flag H-RasV12 expression was induced only in the presence of Cre recombinase (FIG. 4B). These results indicate that pTomo lentiviral vectors are successfully able to induce the expression of H-RasV12 in the presence of Cre recombinase in vitro.

### Example 2

### Assessment of Cre-Inducible Lentiviral Vectors, pTomo In Vivo

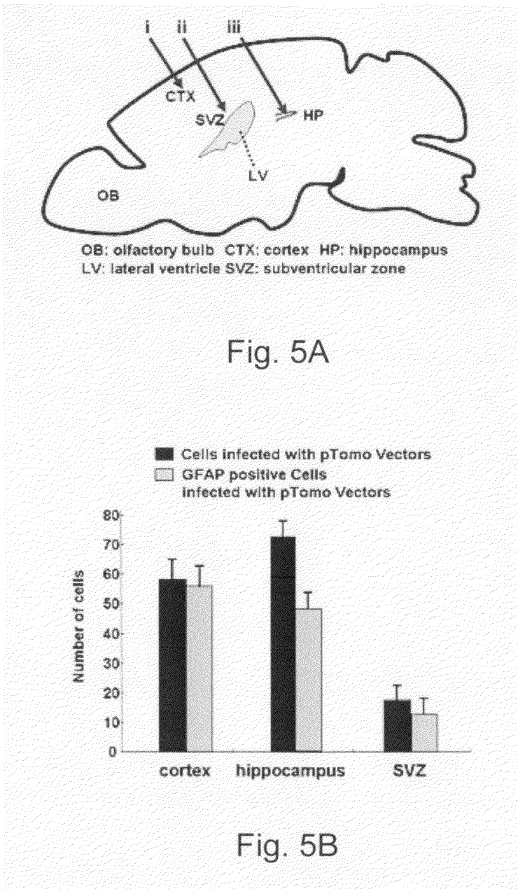

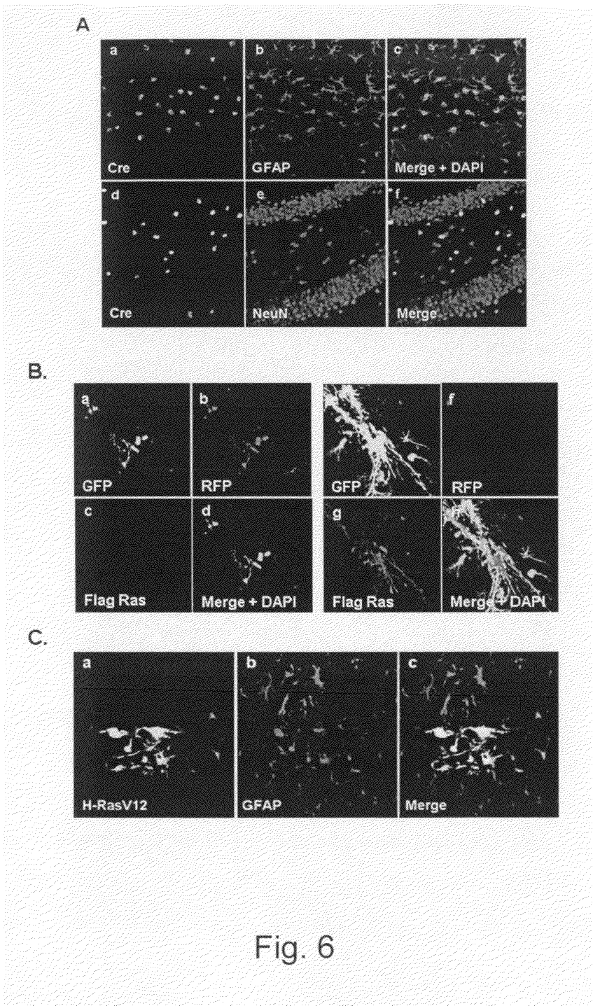

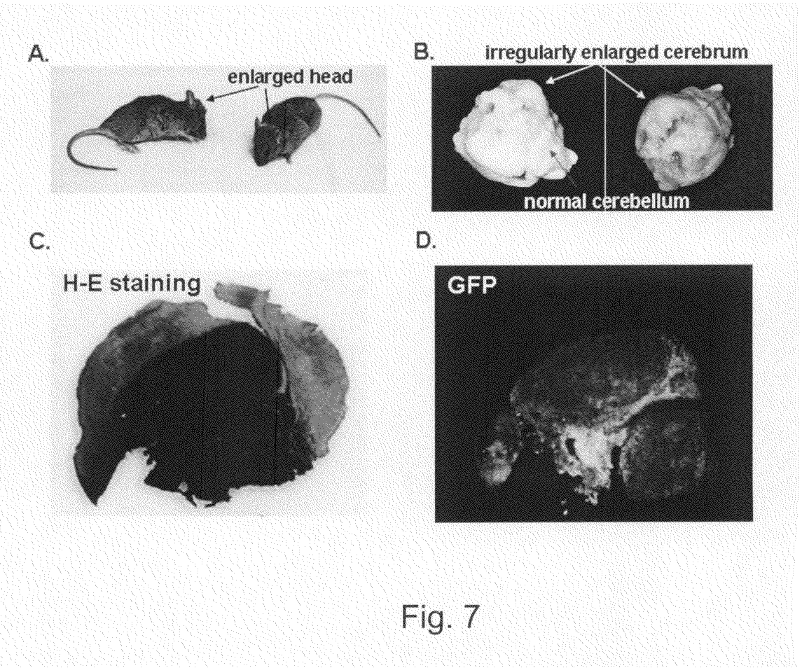

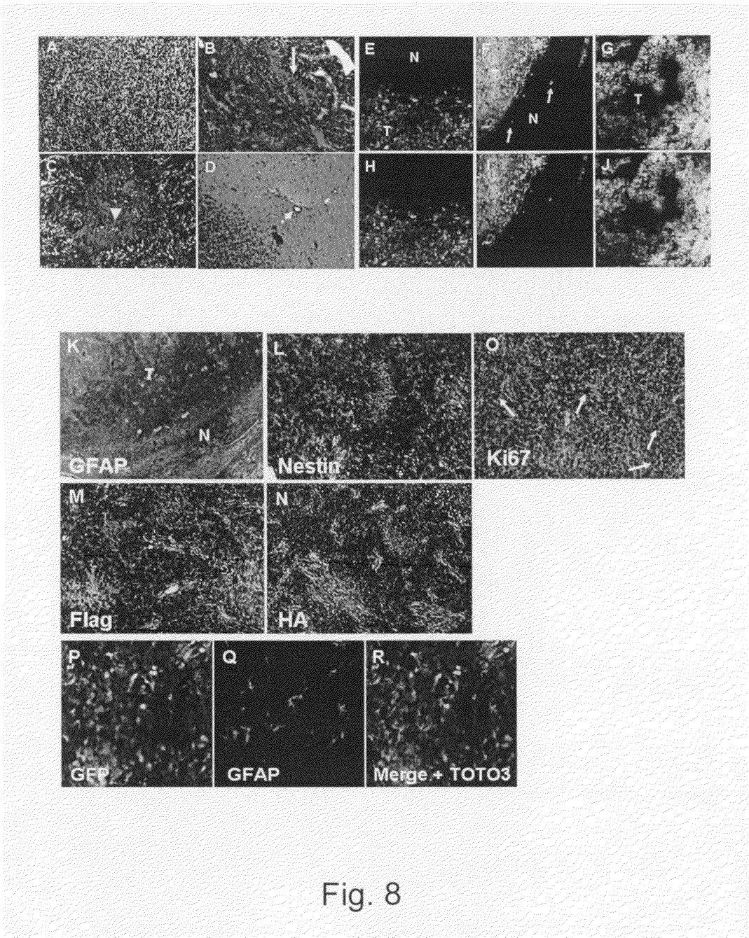

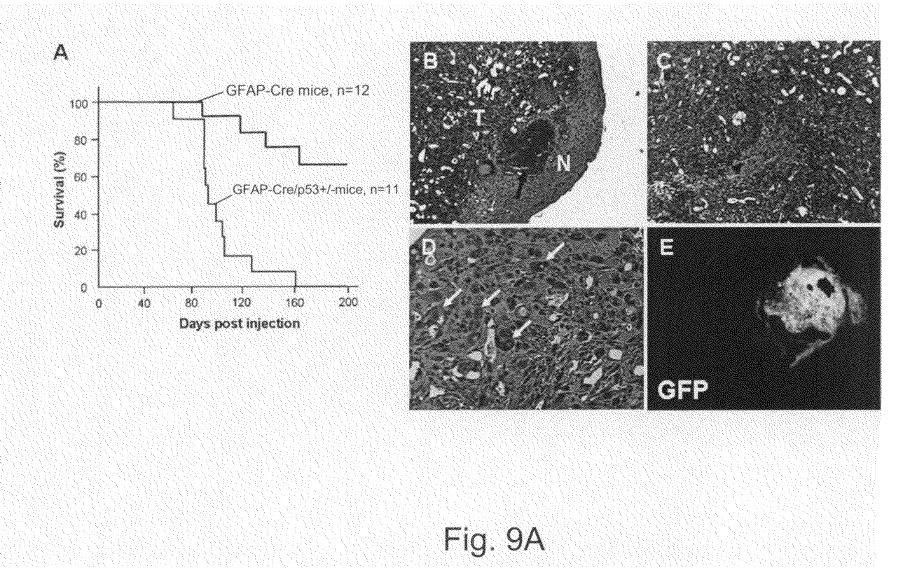

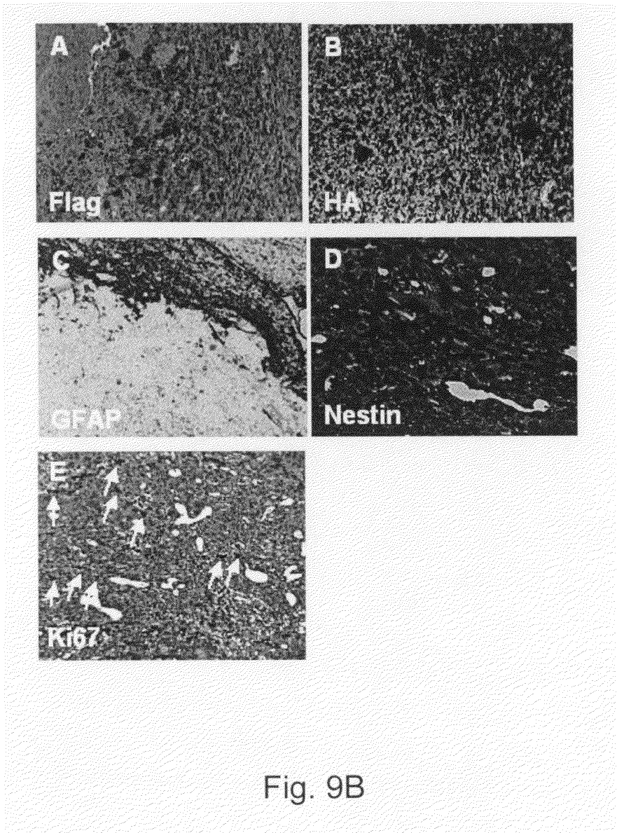

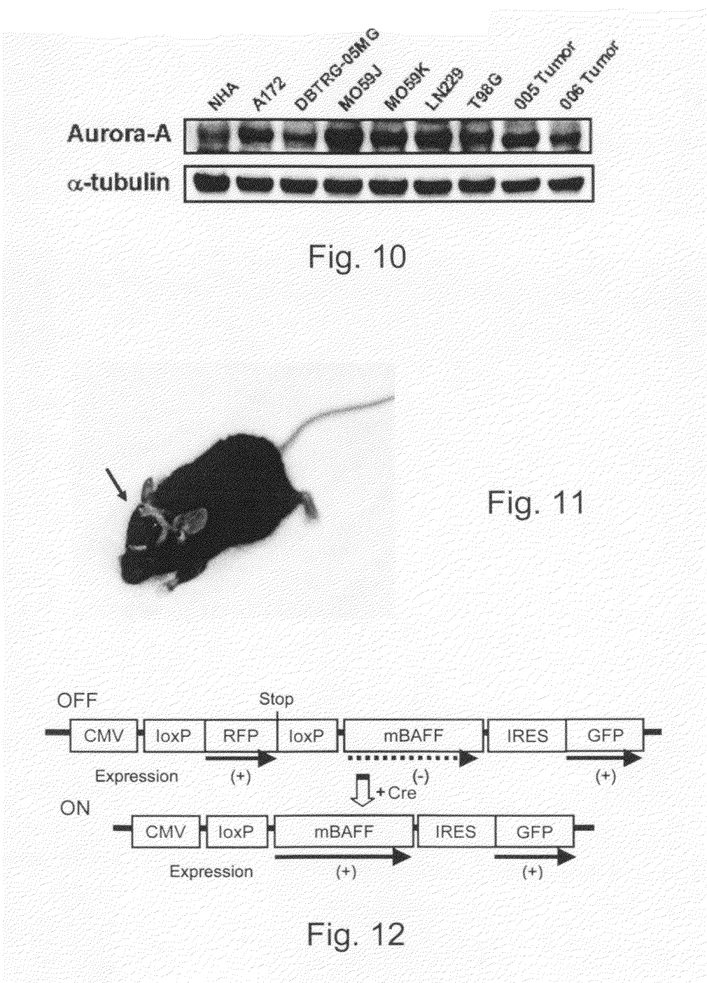

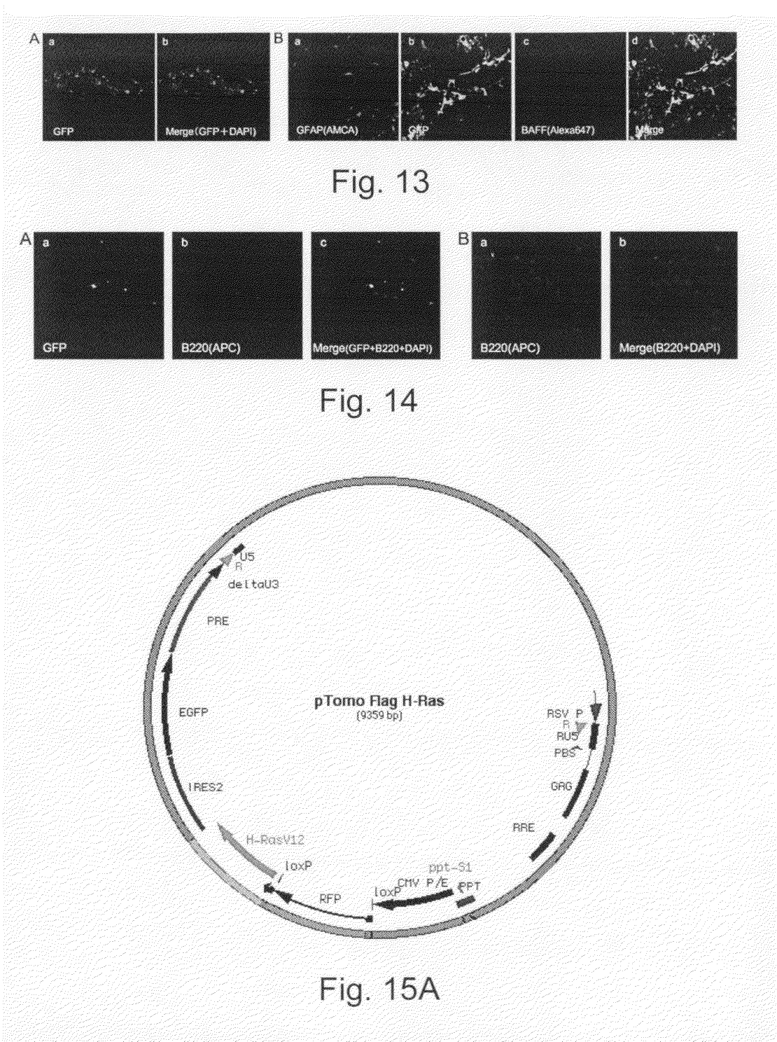

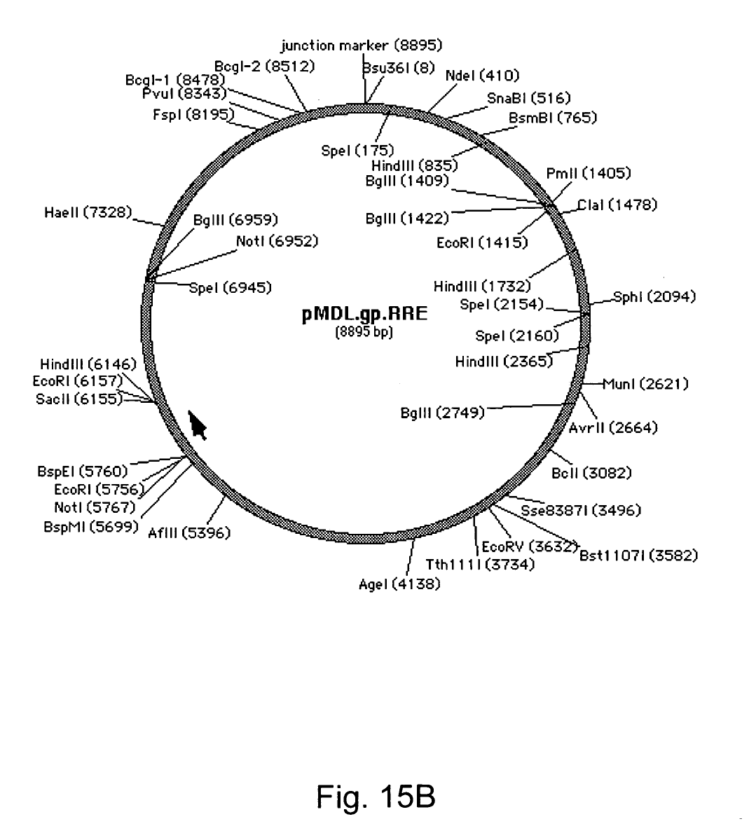

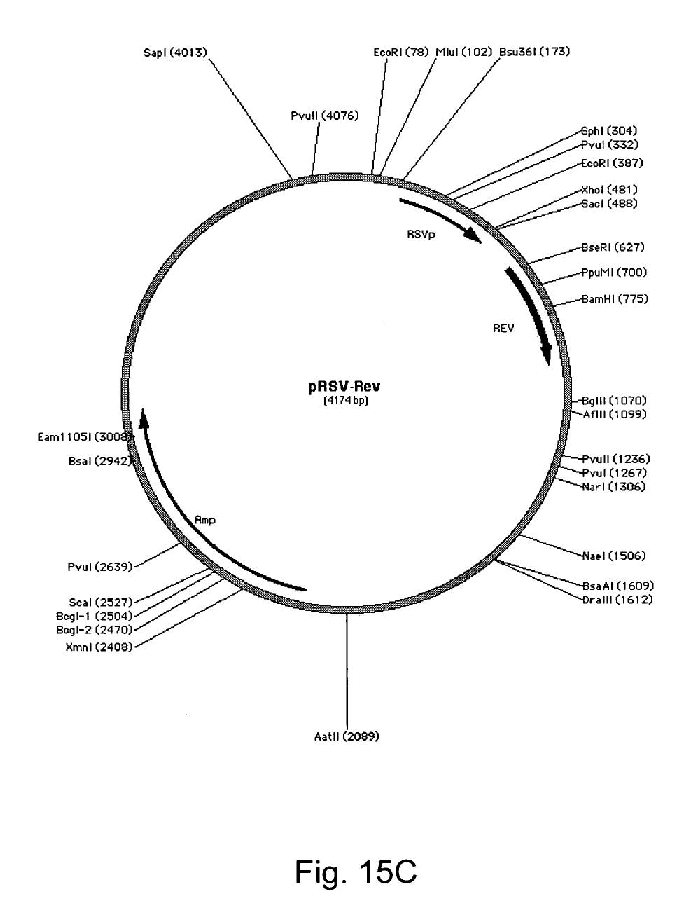

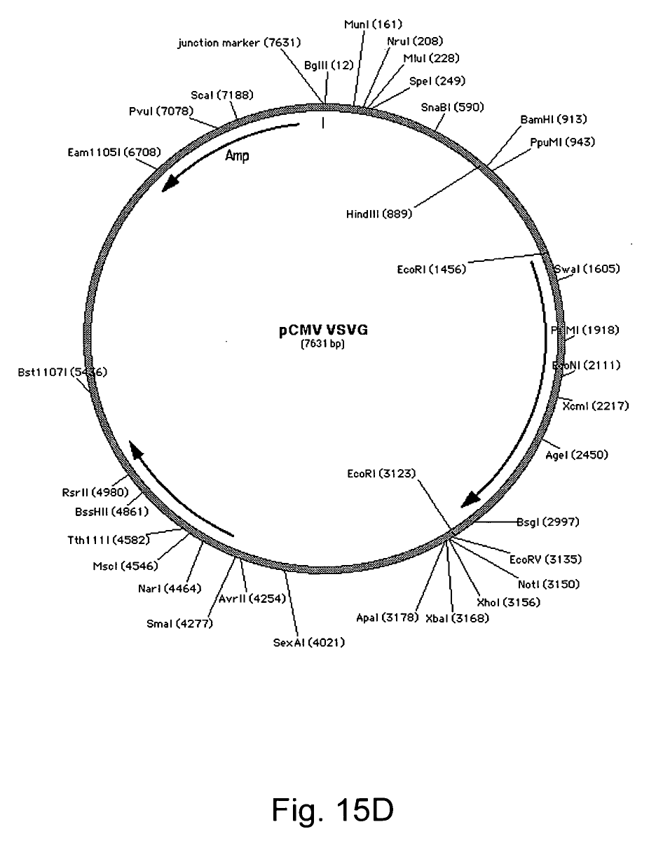

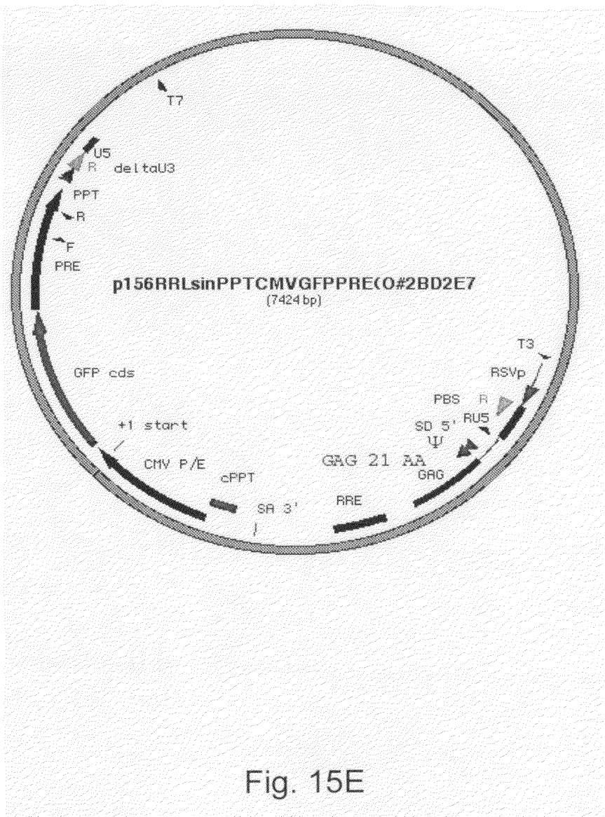

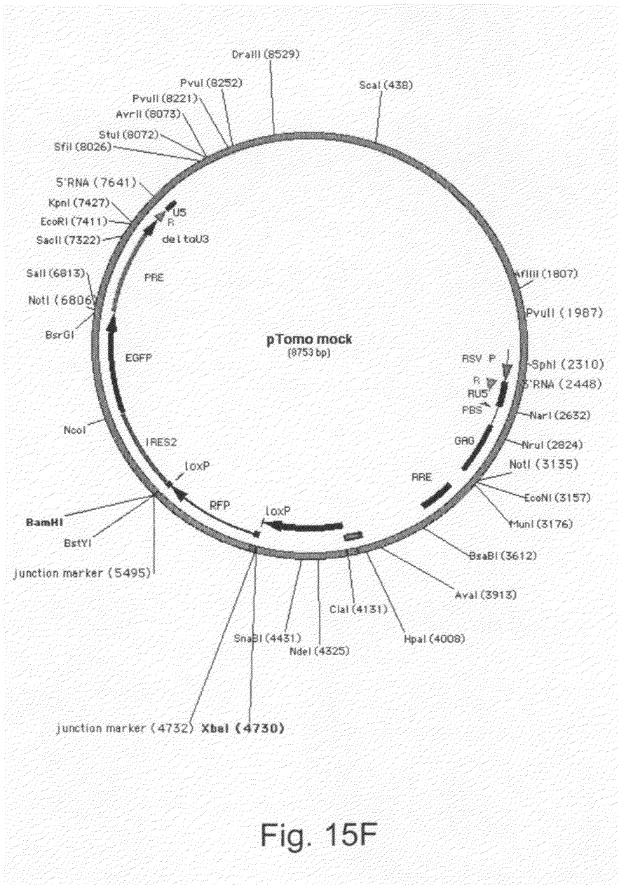

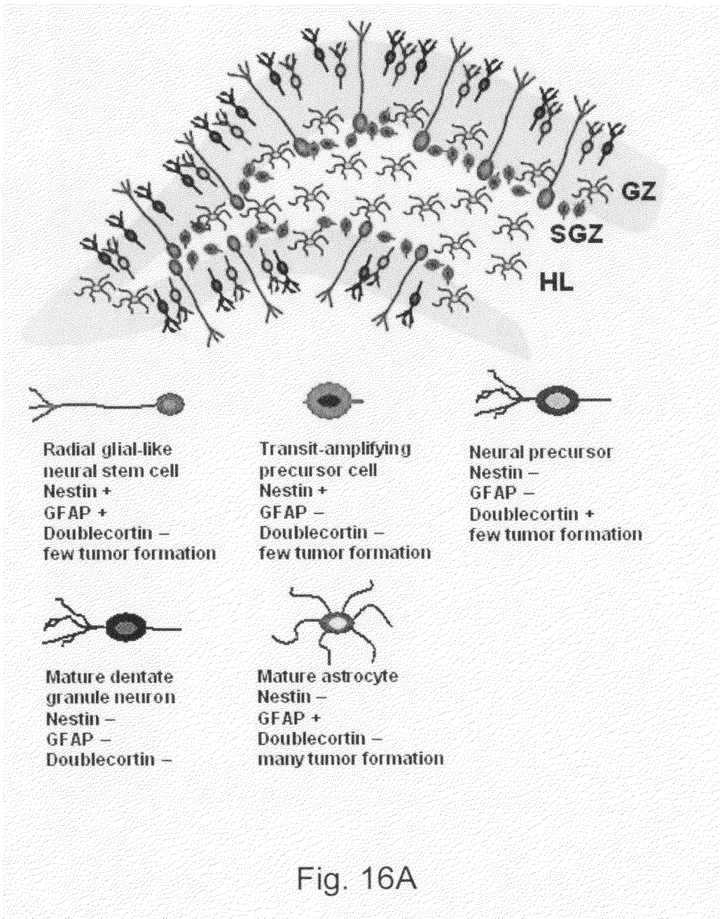

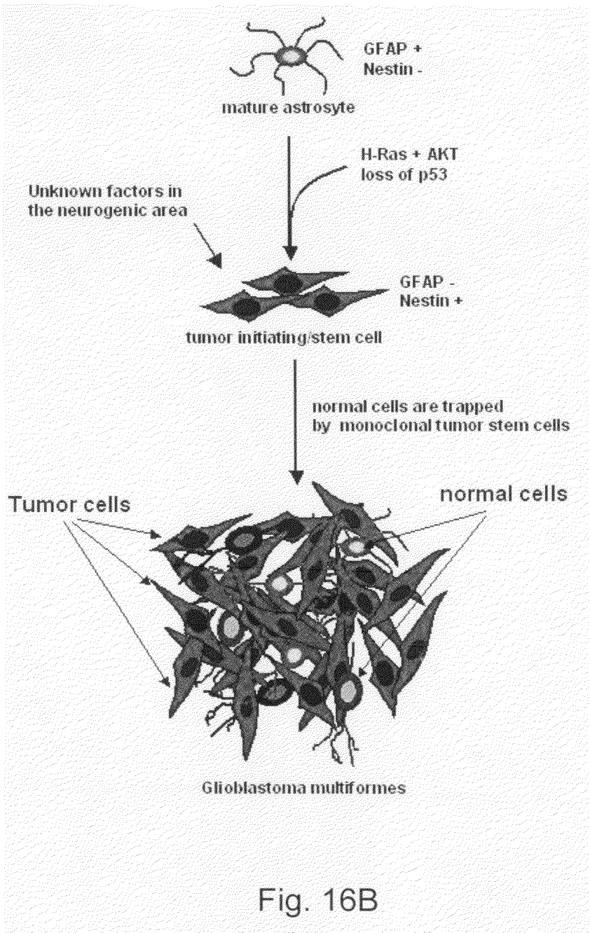

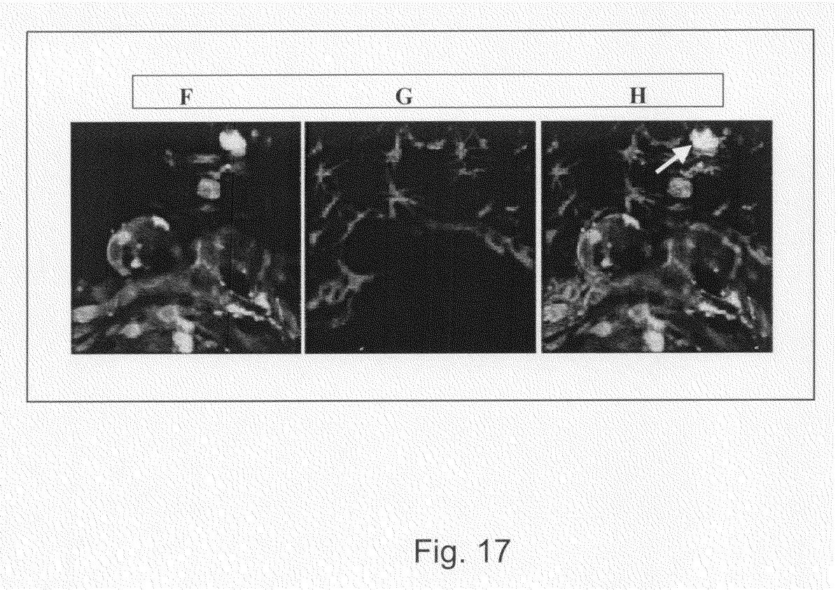

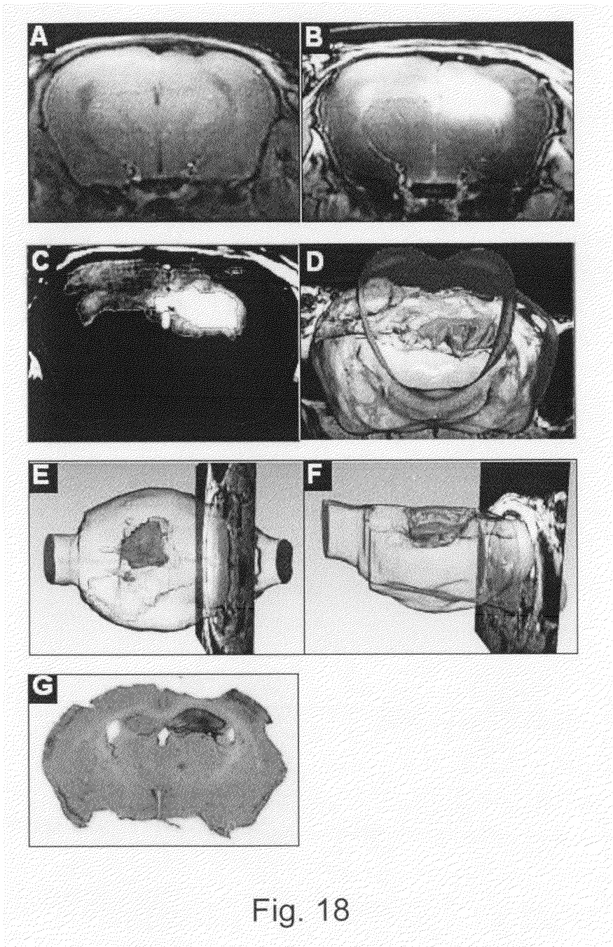

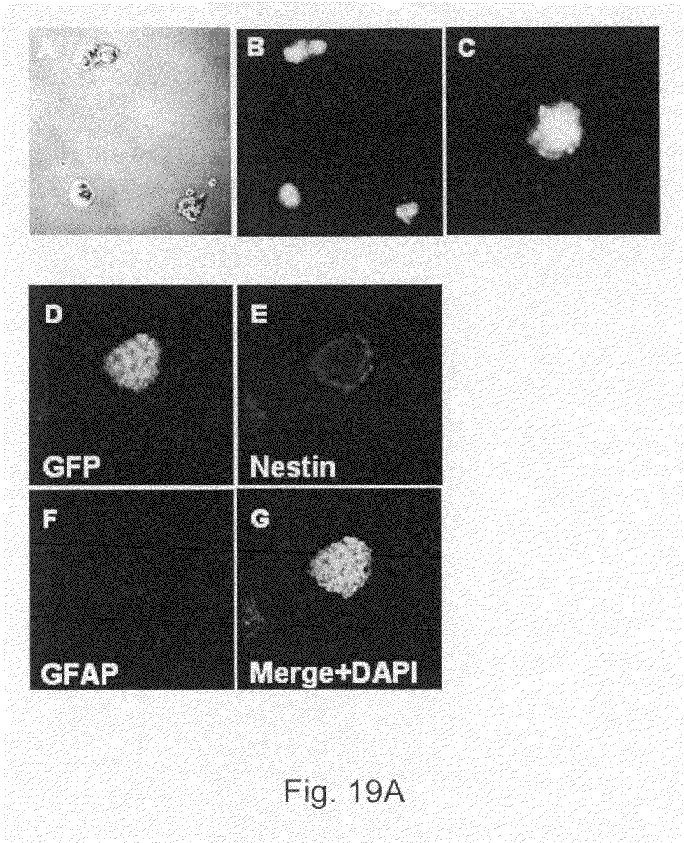

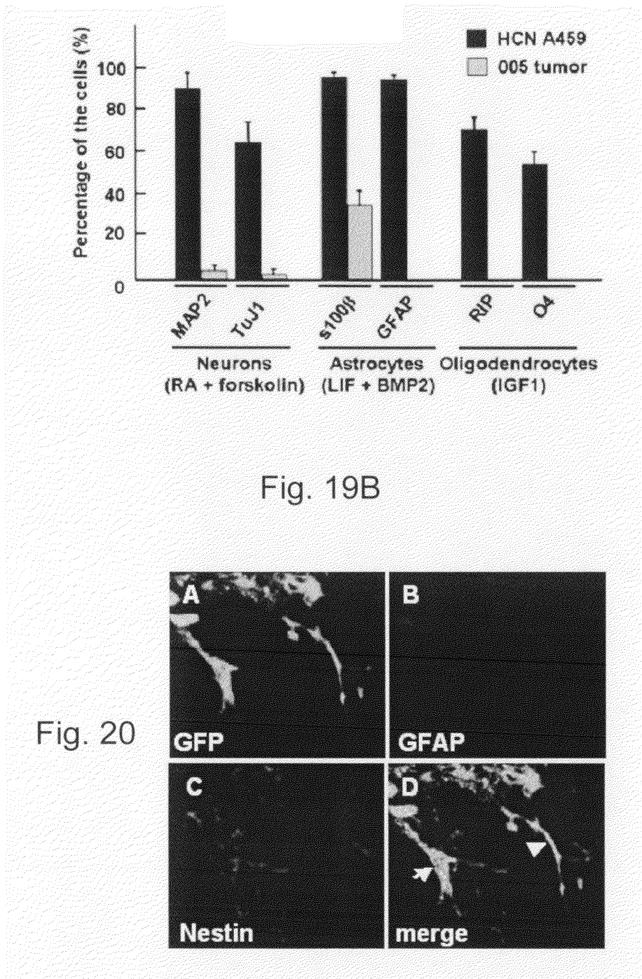

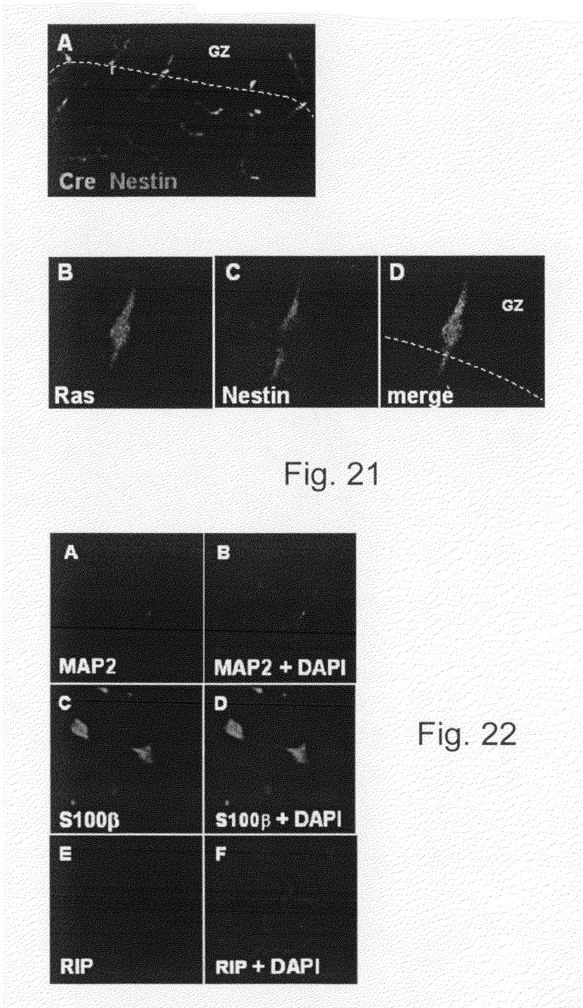

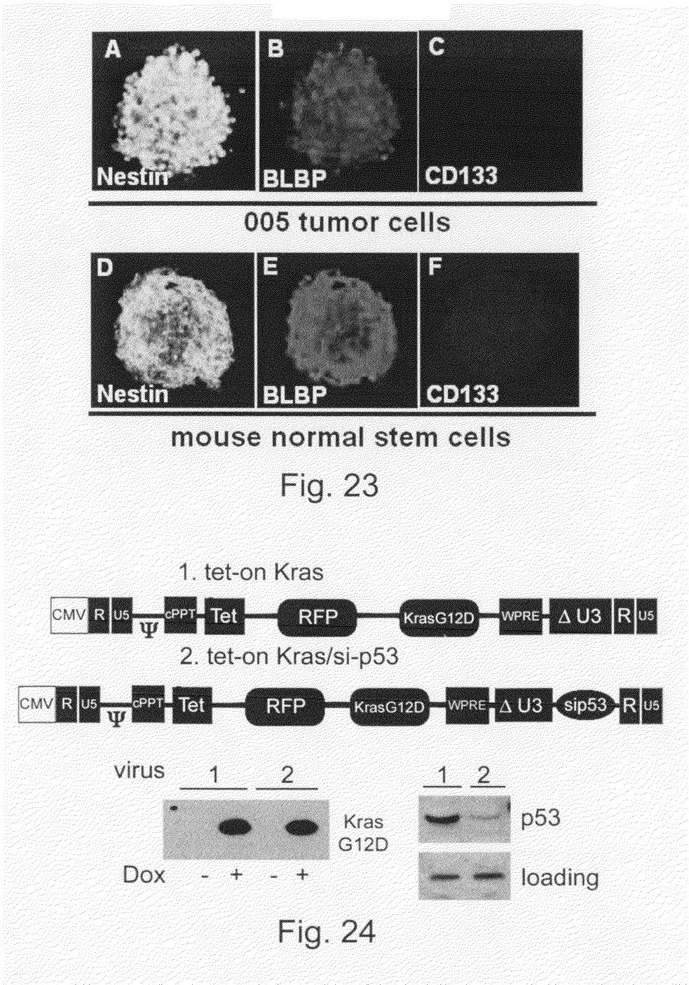

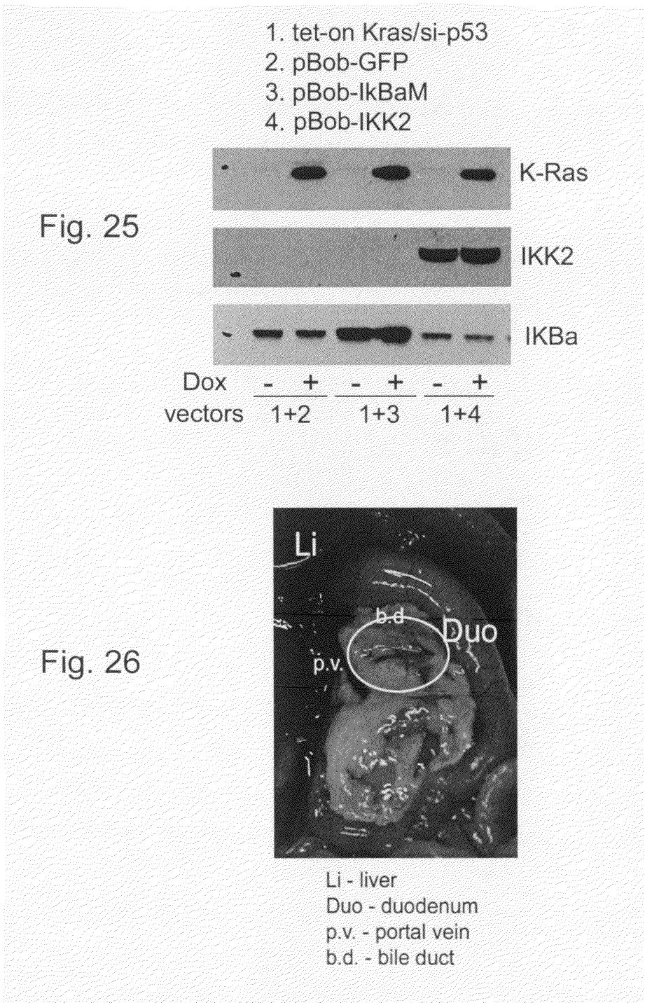

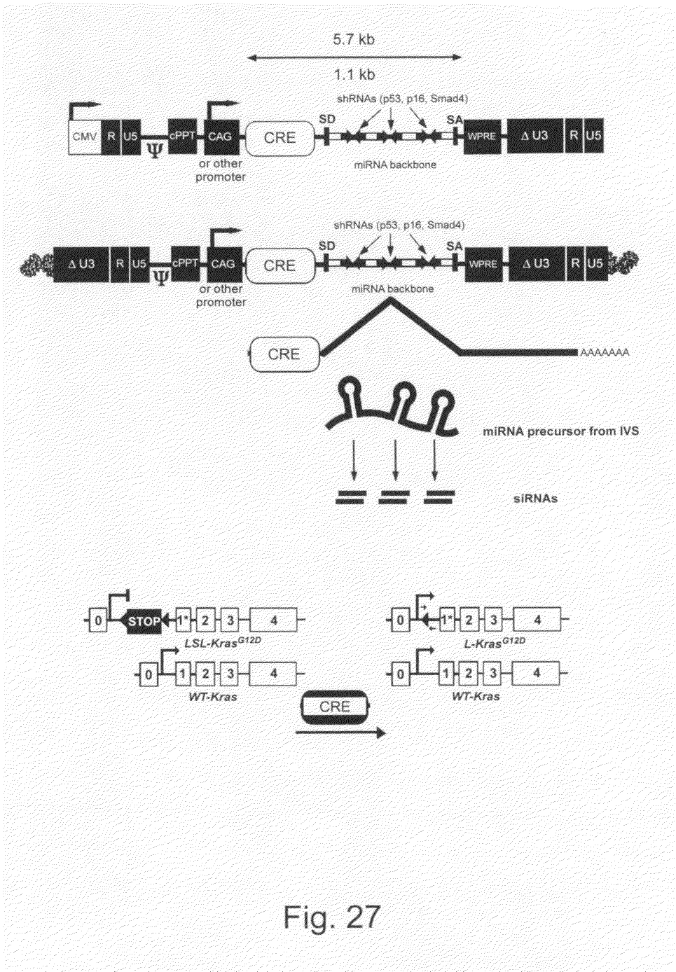

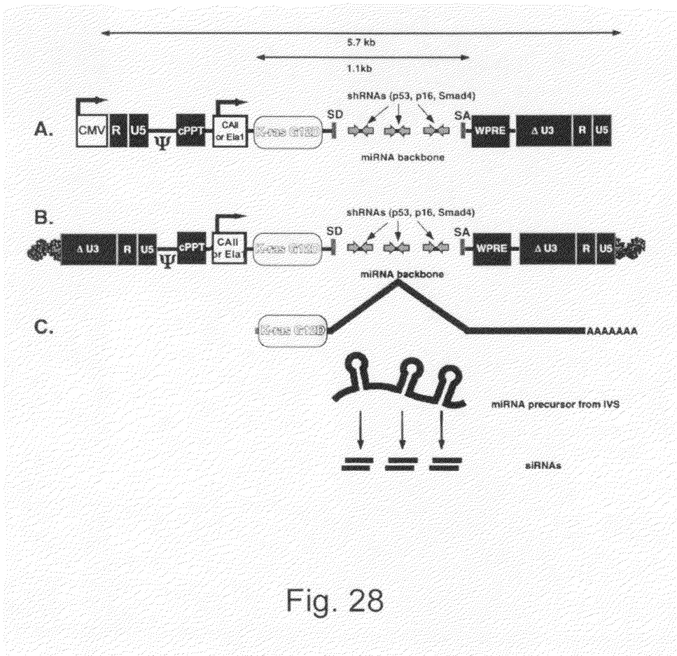

Specific cells within discrete regions of brain, primarily in the dentate gyrus of the hippocampus and in the subventricular zone of the forebrain lateral ventricles, are actively dividing and generating new neurons in adult mice [55]. Thus, these regions are potentially susceptible to cell transformation, which is suggested by the fact that these areas are more sensitive to chemical or viral oncogenesis than are areas with a low population of proliferating cells such as the cortex [56][57][58]. Since it is assumed that the effect caused by the induction of oncogenic event(s) is different depending on the regions induced, we planned to induce oncogenic event(s) in a region specific and a cell type specific manner in an adult mouse brain. To this end, we stereotactically injected pTomo H-RasV12 into the specific locations such as the cortex, subventricular zone or hippocampus in an adult mouse brain that express Cre recombinase under the control of human GFAP promoter (hGFAP-Cre line, FIG. 5A).

It has been shown that Cre immunoreactivity is not present in neurons, ependymal cells, oligodendrocytes, but is detected only in astroglial cells expressing GFAP in the adult CNS of hGFAP mouse line [59]. Consistent with this report, Cre immunoreactivity is not present in neurons (detected by NeuN, FIG. 6A, panels d-f), oligodendrocytes (detected by 04; data not shown), but is detected only in astroglial cells expressing GFAP in the adult CNS of hGFAP-Cre line at the age of 8 weeks old (FIG. 6A, panels a-c).

We stereotactically injected small amount of the viruses (0.8 μl, 1×10⁸ IU) into the right cortex, hippocampus, or subventricular zone slowly (0.1 μl/30s) to minimize the damage to the brain caused by the procedure, and 7 days after the injection, the brains were perfused, removed, fixed, sectioned, and carefully examined to determine the number of cells infected with the viruses at each location. We found that 58.4±7.2 cells and 72.8±5.7 cells were infected with pTomo H-RasV12 vector in the cortex and in the hippocampus respectively, and among them 56.2±7.2 cells and 47.2±5.7 cells were positive for GFAP in the cortex and in the hippocampus respectively (FIG. 5B). However, it was technically difficult to establish equivalent efficiency of the viral infection in the subventricular zone (17.6±5.2 cells were infected with the vectors and 11.8±5.4 cells were positive for GFAP) even though we changed the amounts, concentration and the point of the injection.

Next, we examined if Cre inducible lentiviral vectors (pTomo H-RasV12) are able to express H-RasV12 in the mouse brain expressing Cre in a cell type specific manner. We stereotactically injected 0.8 μl of pTomo Flag H-RasV12 (See plasmid FIG. 15) lentivirus (1×10⁸ IU) into the right cortex, hippocampus or subventricular zone in GFAP-Cre mice, and 7 days after the injection, the brains were perfused, removed, fixed and sectioned followed by immunohistochemistry. As expected, confocal analysis showed that H-RasV12 was expressed only in GFP+/RFP-cells (FIG. 6B). Furthermore, the expression of H-RasV12 was induced only in GFAP-positive cells (FIG. 6C). There was no H-RasV12 expression in NeuN (neuron marker) (FIG. 6C), or 04 (oligodendrocyte marker) positive cells (data not shown). These results indicated that pTomo vectors are able to induce the expression of H-RasV12 only in GFAP positive cells which express Cre recombinase in vivo.

### Example 3

### Induction of H-Ras and AKT in Hippocampal GFAP Positive cells Causing Formation of Human Glioma Like Tumor

We injected pTomo H-RasV12 vectors into cortex, hippocampus or subventricular zone on the right side in GFAP-Cre mice. About 10 months after the injection, all the mice were euthanized and their brains were examined with histological analysis (see, Table 1, below). Since none of the mice developed tumors, we concluded that activation of Ras pathway alone in GFAP-positive cells in each location is not enough to form glioma, which is consistent with a previous report [44]. Loss of tumor suppressor PTEN is often found in glioma and consequently AKT pathway is activated [14][13][60]. We next tried to activate AKT signaling by injecting pTomo AKT (HA tagged activated form of AKT) into the cortex, the hippocampus or the subventricular zone in the right side in GFAP-Cre mice. However there was no tumor formation caused by activated AKT consistent with previous results [44]

TABLE 1

| | location of | | | duration of |
|---|---|---|---|---|
| strain | the injection | Oncogene(s) | tmor incidence | observation |
| GFAP-Cre | CTX | Mock | 0/10 | 10 months |
| GFAP-Cre | SVZ | Mock | 0/10 | 10 months |
| GFAP-Cre | HP | Mock | 0/10 | 10 months |
| GFAP-Cre | CTX | H-Ras | 0/10 | 10 months |
| GFAP-Cre | SVZ | H-Ras | 0/10 | 10 months |
| GFAP-Cre | HP | H-Ras | 0/10 | 10 months |
| GFAP-Cre | CTX | AKT | 0/9 | 10 months |
| GFAP-Cre | SVZ | AKT | 0/10 | 10 months |
| GFAP-Cre | HP | AKT | 0/9 | 10 months |
| GFAP-Cre | CTX | H-Ras + AKT | 0/12 | 10 months |
| GFAP-Cre | SVZ | H-Ras + AKT | 0/12 | 10 months |
| GFAP-Cre | HP | H-Ras + AKT | 5/12 | 10 months |

Mock: empty vector.
CTX: cortex.
SVZ: subventricular zone.
HP: hippocampus

In gliomas, growth factor receptors such as EGFR are activated which result in the activation of downstream signaling such as Ras and AKT pathways. Thus, we next activated both Ras and AKT signaling in the adult mouse brain in a region/cell type specific manner. 50 out of 50 cells infected with both pTomo H-RasV12 and pTomo AKT showed effective induction of both H-RasV12 and AKT in each cell. Interestingly, approximately 4-5 months (136±32 days) after the injection, we found that 5 out of 12 mice injected with pTomo H-RasV12+pTomo AKT into the right hippocampus in GFAP-Cre mice showed enlarged head and lethargy (FIGS. 7A and 7B), whereas neither GFAP-Cre mice injected with these vectors in the cortex, subventricular zone nor GFAP-Cre mice injected with mock vectors showed tumor formation. The brains of five of these mice showed parenchymal massive lesions that vary in size. The gross appearance of the lesions was very similar to that seen with human gliomas. These lesions were characterized microscopically by lesions of increased cell density, but mitotic figures and nuclear pleomorphism were not so obvious (FIG. 8 A-D). Microvascular proliferation was present both within the tumor as well as in the surrounding brain parenchyma. Serpiginous zones of tumor necrosis bordered by dense palisades of viable tumor cells (necrosis with pseudopalisading) which is one of the most characteristic features of human GBMs was also seen in these tumors (FIG. 8B). These histological features were present in all tumors examined (5 out of 5).

pTomo vectors are able to express GFP under the control of IRES (FIG. 4). Interestingly, more than 90% of the cells within the tumor area expressed GFP (FIG. 7D), suggesting that most of the tumor cells arose from the cell of origin and less than 10% of the cells were adventitiously taken up by the tumor. The tumor also contained heterogeneous area (FIGS. 8G and 8J). Moreover, the border between the GFP-positive tumor cells and normal tissues were relatively clear (FIGS. 7D, 8E and 8H), however GFP-positive tumor cells sometimes invaded the normal tissues (FIG. 8F, white arrows), which suggests the tumor has a malignant invasive character. This also reemphasizes the role of normal surrounding cells in expansion of malignancy, a unique feature of this technology, as compared to traditional transgenics where all the cells in the tissue have an oncogenic event.

### Example 4

### Tumors Induced by Active H-Ras and AKT in GFAP-Cre Mice were Positive for Nestin but Negative for GFAP

To further characterize the tumor found in GFAP-Cre mice injected with pTomo H-RasV12 and pTomo AKT into the right hippocampus, immunohistochemistry experiments were performed. H-RasV12 and AKT expression was observed in tumor tissues (FIGS. 8M and 8N). More than 90% of the tumor cells within the tumor were GFP positive (FIG. 7D), suggesting that most of the tumor cells were derived from the small number of the cells infected with pTomo vectors in the hippocampus. Surprisingly, most of the cells in the tumor were GFAP-negative (FIG. 8K), although H-RasV12 and AKT were induced specifically in GFAP positive cells. Moreover, A small population of GFAP positive cells within the tumor were GFP negative (FIG. 8 P-R), suggesting that GFAP+/GFP− cells in the tumor were trapped normal astrocytes. Furthermore, most of the tumor cells were Nestin-positive (FIG. 8L; showing staining in rough V-shaped portion of panel image), although we induced oncogenic events in GFAP-positive cells in the hippocampus. All the tumors examined (n=5) showed similar results. Considering that human malignant gliomas often lack the GFAP expression and show nestin expression as they progress their malignant phenotypes [61], this mouse glioma model may recapitulate well what actually happens in human glioma formation. Ki67 staining which indicates the mitotic activity of the tumor showed relatively low population of cycling cells (FIG. 8O; showing rare stained cells, e.g., at arrows).

Again, induction of activated H-Ras and AKT in the hippocampal GFAP positive cells caused the formation of malignant glioma characterized by increased cellular density, necrosis, and tumor invasion into the surrounding normal tissues. However, the tumor lacked the prominent nuclear pleomorphism, giant cell formation and increased mitotic figures (FIGS. 8 A-D and O).

### Example 5

### Loss of p53 Shortened Tumor Latency and Modulated Cell Morphology

It has been suggested that loss of p53 plays a role in malignant transformation of gliomas [62-64]. These findings were based on the difference of the genetic alterations between low grade gliomas and their transformation to malignant gliomas. However the precise effect of loss of p53 on the histopathology of gliomas has not been shown clearly using mouse models and remains vague. To clarify this, we injected pTomo H-RasV12 and pTomo AKT vectors into the brains of the GFAP-Cre mice on p53+/− genetic background. Surprisingly, 100% of the mice (11/11) injected with pTomo H-Ras and AKT into the right hippocampus showed tumor formation (Table 2, below). The tumor latency in GFAP-Cre/p53+/− mice was much shorter than that in GFAP-Cre mice injected with H-Ras and AKT expressing viruses (FIG. 9A). Histological analyses revealed the lesions characterized by high cellular density, necrosis within dense cellular lesion, intratumoral hemorrhage, nuclear pleomorphism and high mitotic activity, all of which are the characters seen in human GBMs (FIG. 9, B-D). It is notable that giant cell formation, nuclear pleomorphism and mitotic activity were much more obvious in the tumor found in GFAP-Cre/p53+/− mice compared with that in GFAP-Cre mice injected with ptomo H-RasV12 and AKT (FIG. 9, B-D). Expression of H-Ras and AKT was found in the tumor and the tumor was GFAP negative and Nestin positive as same as the tumor found in GFAP-Cre mice. Expression of both H-Ras and AKT was readily detected in the tumor, and furthermore the tumor was GFAP−/nestin+as found in H-RasV12 and AKT induced tumors (FIG. 17, panels F-H and FIG. 9B, panels C and D). As with activated H-Ras+AKT induced tumors (FIG. 7D), the tumors in the p53+/− genetic background are also largely GFP+ (FIG. 9E). Some tumor cells migrate to neighboring non-tumor tissue (FIG. 17, panels F-H) indicative of strong infiltrative character of the tumor cells.

| | location | | | |
|---|---|---|---|---|
| | of the | | tmor | duration of |
| strain | injection | Oncogene(s) | incidence | observation |
| GFAP-Cre/p53+/− | CTX | Mock | 0/9 | 10 months |
| GFAP-Cre/p53+/− | SVZ | Mock | 0/10 | 10 months |
| GFAP-Cre/p53+/− | HP | Mock | 0/10 | 10 months |
| GFAP-Cre/p53+/− | CTX | H-Ras + | 1/11 | 10 months |
| | AKT | | | |
| GFAP-Cre/p53+/− | SVZ | H-Ras + | 1/10 | 10 months |
| | AKT | | | |
| GFAP-Cre/p53+/− | HP | H-Ras + | 11/11 | 10 months |
| | AKT | | | |

Mock: empty vector.
CTX: cortex.
SVZ: subventricular zone.
HP: hippocampus

Our results indicate that loss of p53 is responsible for the various histological characters such as giant cell formation, nuclear pleomorphism and high mitotic activity during the development of GBMs. This is the first report using mouse models to show that loss of p53 plays a role in the malignant transformation of gliomas in adult mice.

Identification of mice with brain tumors is difficult due to their location within the cranial cavity. Non invasive imaging techniques like magnetic resonance imaging (MRI) are being used for screening and detecting brain tumors in mice. Furthermore high-grade malignant gliomas show contrast enhancement after the injection of gadolinium, thus indicating the break down of blood-brain barrier, a diagnostic feature of GBMs. FIG. 18 shows MRI images of tumors induced by combined activation of H-Ras and AKT in p53+/−genetic background. There are clear areas of enhanced contrast following i.p. injections of gadolinium (FIG. 18, panels B and C) implying the break down of blood-brain barrier. H-E staining of the tumor (FIG. 18 panel G) indicates that the relative intensity in the shade coded (tumor being represented by the grayer zones) 3D model reflects the extent and distribution of the tumor cells in the brain (FIG. 18, panels D-F).

### Example 6

### Aurora-A Kinase is Overexpressed in GBM Cell Lines and Tissues

To examine whether Aurora-A is overexpressed in GBMs, we first performed western blot analysis using the lysates from six human GBM cell lines and two mouse GBMs cell lines developed from tumors described above. We found that all the cell lines examined showed higher expression of Aurora-A compared with control normal human astrocytes (FIG. 10). Consistent with this data, 5 out of 8 human GBM tissues showed the high expression of Aurora-A, although not all the tumor cells expressed it. In addition, NFκB is activated in glioma tissues showing Aurora-A expression (data not shown).

### Example 7

### Overexpression of Aurora-A in GFAP Positive Cells Caused Brain Tumor Formation

To examine whether Aurora-A causes the formation of gliomas, we injected pTomo Aurora-A into the right hippocampus in GFAP-Cre mice on the p53−/− genetic background at the age of 8 weeks, and observed for about 4 months until they died due to leukemia. Although none of 5 control GFAP-Cre/p53−/− mice injected with pTomo mock vectors showed brain tumor formation, one out of 5 GFAP-Cre/p53−/− mice injected with pTomo Aurora-A showed brain enlarged head (FIG. 10) indicating brain tumor formation.

In summary, a novel brain tumor model has been generated that recapitulates the pathology of human gliomagenesis in adult humans. We have further shown that Aurora-A kinase is overexpressed in gliomas. Moreover, Aurora-A kinase may activate NFκB pathway which is known to play a role in gliomagenesis. Therefore, we feel that we are in an excellent position to understand the molecular mechanisms by which Aurora-A kinase and NFκB proteins are involved in glioma formation and progression. These examples of how to prepare more representative tumor models, provides ways, e.g., to screen drugs for treatment or explore new strategies of treating these deadly diseases.

### Example 8

### A Mouse Model for Primary CNS Lymphoma

Primary central nervous system lymphoma (PCNSL) is an extranodal non-Hodgkin's lymphoma (NHL), which arises in the central nervous system (CNS) in the absence of systemic lymphomas. The incidence rates of this disease in those age 60 and older have increased nearly 10-fold in past 2 decades due to unknown reason. Regardless of intensive chemo-radiotherapy, the prognosis of these patients is very poor. Therefore, there is an urgent need to elucidate the mechanism to cause the formation of this disease and develop the novel strategy of the treatment. Although the etiology of the PCNSL is not well understood, it is conceivable that the abnormal microenvironment of the brain plays a role in the formation of PCNSL. It has recently been shown that B-cell activating factor of the TNF family (BAFF) is secreted from astrocytes in response to inflammation in brain, and the PCNSL cells also express BAFF receptors. Therefore, we hypothesize that BAFF secreted from astrocytes may trigger the transformation of B cells which had accumulated in brain by the prior inflammation.

One of the major reasons for the lack of clinical advances in the PCNSL for decades is lack of sufficient knowledge about lymphomagenesis. The model systems of the invention can be useful in clarifying the relevance of BAFF and astrocytes to the PCNSL. A novel mouse model for the PCNSL can be generated with astrocyte-specific BAFF expression using lentiviral vectors and transgenic mouse technology. For example, according to FIG. 12, mouse BAFF cDNA was inserted in a Cre-inducible lentiviral vector plasmid pTomo just behind a loxP-stuffer-STOP-loxP cassette. In the presence of Cre, cells transduced with this plasmid or lentiviral vector derived from this plasmid (Tomo-BAFF LV) will express GFP and BAFF.

The Tomo-BAFF-LVs were produced by transfection of pTomo-BAFF along with package plasmids as reported previously (Follenzi A. et al. Nat Genet. 2000). In GFAP-Cre mice injected with this LVs in their brain hippocampus stereotaxically, we could confirm successful BAFF expression in the GFAP-positive cells in the hippocampus (FIGS. 13A and 13B).

To make astrocytes produce BAFF, Tomo-BAFF LVs were injected in the hippocampus of GFAP-Cre mice. Because, an initiation event might be required in tumor generation step, we also injected the Tomo-BAFF LVs into some oncogenic mice with GFAP-Cre transgene; i.e. GFAP-Cre/p53+/− and GFAP-Cre/p16+/− mice. After 5 months from injection, an accumulation of B cells was detected the in the injected site of hippocampus (FIG. 14). Particularly, in the GFAP-Cre/p16+/− mice, a typical finding of the PCNSL was seen in the accumulation of B cells surrounding blood vessels.

Development of new therapeutic strategies for these lymphomas is difficult because etiology of the PCNSL is still unclear and there is no good animal model. This mouse model is the first PCNSL model besides NOD-SCID mouse model transplanted with patient's lymphoma cells intracranially. Because this mouse model is uniquely reproducing the microenvironment for the generation of PCNSL, model will provide much information about the lymphomagenesis and the nature of the PCNSL. Specific characteristics of the lymphoma cells such as BAFF and astrocyte dependency and activated pathways can become target points for screening new therapeutic agents. In addition, this mouse model can be useful in preclinical in vivo study of new therapeutic modalities for the PCNSL. Moreover, the knowledge obtained from this mouse model may also be useful for the secondary CNS lymphoma.

### Example 9

### Brain Tumor Initiating/Stem Cells in this Model

Normal neural stem cells may be isolated in culture as clonally derived colonies termed neurospheres. Here, we established primary cultures of the brain tumor induced by the combined activation of H-Ras and AKT in GFAP-Cre/p53+/−mice, and evaluated the cultures to see if the tumor contains brain tumor initiating/stem cells (BTICs). The tumor cells were cultured in the normal medium for astrocytes (DMEM-F12+10% FBS) or in the medium prepared for neural stem cells (DMEM-F12+N2 supplement+epidermal growth factor: EGF+fibroblast growth factor: FGF+heparin). The tumor cells in the normal medium survived for 1 week but eventually died within 2 weeks, whereas the tumor cells in the medium prepared for culturing neural stem cells could survive for more than 10 passages. Furthermore, GFP positive tumor cells cultured in the medium prepared for neural stem cells formed neurosphere-like structure (here on termed tumorsphere, FIG. 19A, panels A and B).

To examine the self-renewal capacity of GFP+tumorsphere-forming cells, we dissociated primary tumorspheres and replated single-cell suspensions of these cells at serial dilutions down to one cell per well. Primary tumorsphere-derived GFP+ cells formed secondary spheres (FIG. 19A, panel C) with a range of 76 to 88 secondary sphere/100 primary sphere cells (mean 81.3±5.1) over a period of 1-2 weeks, indicating that most of the tumor cells have the ability to self-renew. This high percentage of cells that can form secondary spheres in the tumor was unexpected because previous reports suggested that only a small population of the brain tumor cells has the ability to self renew. Since over 90% of the tumor cells were GFP positive in our mouse model (FIG. 7, panel D and FIG. 9, panel E) it will appear that a vast majority of the tumor cells have the potential to be BTICs. In keeping with the data shown in FIG. 9B (panels C and D) with primary tumors, the tumorspheres are also nestin+/GFAP− (FIG. 19A, panels D-G). These results prompted us to examine if GFP+/nestin+ tumor cells are BTICs.

To examine this possibility, we first tested if GFP+/nestin+ tumor cells have the ability to differentiate in response to various stimuli. GFP+ tumor cells (005 tumor cells which were freshly obtained from the tumor in GFAP-Cre/p53+/− mice) were sorted by FACS, cultured in the medium containing 10% FBS without FGF, EGF and heparin for 6 days and the cells were fixed and stained with various markers. The tumor cells still expressed nestin but did not show the expression of any markers including GFAP, s100b, MAP2, NeuN, RIP and O4 (data not shown). We next plated the tumor cells (GFP+/nestin+005 tumor cells) on the dish coated with poly-L-ornithine and laminin, and cultured in the medium containing various factors (forskolin and retinoic acid for neural differentiation, BMP2 and LIF for astrocytic differentiation, and IGF1 for oligodendrocytic differentiation) for 6 days. This is a reliable method to examine the differentiation of normal neural stem cells into the specific lineages. As shown in FIG. 19B, normal rat neural stem cells (HCN A459 cells) differentiated into neurons, astrocytes and oligodendrocytes in response to various stimuli. In contrast, 005 tumor cells did not express any differentiation markers in the presence of various stimuli, though 35.0±8.5% of 005 tumor cells became positive for s100 b in response to LIF and BMP2, but were negative for GFAP (FIG. 19B and FIG. 22). These results indicate that 005 tumor cells remain in an undifferentiated state in the presence of these stimuli but have the ability to self renew.

It has been shown that most xenograft models for brain tumor using human tumor cell lines require at least 10⁵ cells, whereas only 100 BTICs are enough for the tumor formation in the immunodeficient mouse brain. Thus, we next examined whether 005 tumor cells can form brain tumor when injected into the brain of NOD-SCID mice. Injection of only 100 of 005 tumor cells into the hippocampus of NOD-SCID mice showed that 78% (7/9, average latency 40.3±6.0 days) of the mice formed tumors whereas no control mice (0/8) injected with 100 mouse normal stem cells (kindly provided by Dr. Fred H gage) showed any tumors (data not shown). Moreover, injection of only 10 cells into the hippocampus in NOD-SCID mice formed tumors in 50% (4/8, average latency: 63.2±6.8 days), indicating the strong tumor forming activity of 005 tumor cells.

The gross appearance of the brain of the injected mice showed a dark area which suggests the massive lesion with hemorrhage (data not shown). The tumor formed in NOD-SCID mice showed histopathology of glioma-like phenotypes including the increased cellular density, necrosis within the dense cellular lesion, intratumoral hemorrhage. Notably, the tumor also showed infiltrative character which is not found in xenograft models using glioma cell lines 31. Unexpectedly, more than 99% of the GFP+/nestin+ tumor cells were negative for CD133, which is currently used as a marker for BTICs (FIG. 23). Therefore, these results suggest that nestin+/CD133-population of the tumor cells can be the BTICs. Since 90% of the cells in the tumor were GFP+, it would appear that the majority of the tumor cells that exist throughout the tumor can be the BTICs in certain types of GBMs. Additionally, given that the GFP+ tumor cells are the descendants of GFAP+ cells in the hippocampus infected with H-Ras and AKT expressing vectors, GFAP+ cells in the hippocampus can be the cellular origin of BTICs in GBMs.

### Example 10

### Transfection of Packaging Cells

I. Plasmids were obtained in the form of Qiagen, endo-free Maxi or Giga preps. Concentration of the plasmids was determined by A260 and quality verified by agarose gel electrophoresis. Identify of the plasmids was verified by confirmation of restriction digestion patterns.

II. Transfection buffers were prepared.

Stock Solutions:

| | |
|---|---|
| 500 mM BES | 10.66 g/100 ml |
| 2.8 M NaCl | 16.35 g/100 ml |
| 150 mM Na2HPO4 | 2.1 g/100 ml |
| 2.5 M CaCl2 | 36.75 g/100 ml |

To make 1 liter 2×BBS combine: 100 ml 500 mM BES, 100 ml 2.8 M NaCl, and 10 ml 150 mM Na2HPO4; titrate to pH 6.95 with 1 M NaOH; and, filter sterilize.

To make a CaCl₂ working solution dilute stock solution 1:10 immediately before use.

III. Vector Particle Production Protocol:
a. Split a confluent 15 cm plate to 12 poly-L-lysine coated plates. To coat plates with poly-L-lysine, prepare 0.01%/5 poly-L-lysine in 1×PBS, filter. Add 10 ml per 15 cm dish, incubate at room temperature for 15 minutes. Aspirate off liquid; no need to dry.
b. Trypsinize thoroughly but not excessively, mix well, swirl vigorously to ensure even coverage. Plate the cells onto the treated plates. When cells reach 70% confluence, proceed to transfection.

c. In a 50 ml tube, mix the 4 plasmids according to the table below:

| | 10 cm plate | 15 cm plate | 12 × 15 cm plates |
|---|---|---|---|
| HIV vector plasmid | 10.0 ug | 22.5 ug | 270 ug |
| PMDL | 6.5 ug | 14.6 ug | 176 ug |
| Rev | 2.5 ug | 5.6 ug | 68 ug |
| VSV-G | 3.5 ug | 7.9 ug | 95 ug |

d. Add 500 ul of 0.25 M CaCl₂ to the DNA mix per 10 cm dish; 13.5 ml for 12 of 15 cm dishes.
e. Add 2×BBS solution to the calcium-DNA mix (same amount as CaCl2).
f. Incubate at room temperature for 15 minutes.
g. Add the DNA buffer mixture dropwise onto dishes (12 plates), 2.25 ml per plate.
h. Incubate the cells in 3% CO₂, at 37° C.
i. Change the medium at 12-16 hours (maximum 24 hrs) post transfection with the 20 ml fresh media per plate. Use DMEM+10% FBS, or 2% FBS or Optimem. We suspect Optimem gives cleaner preps without loss of titer.
j. Collect the medium 3 times at 24 hour intervals beginning 24 hours after changing the medium post-transfection. Alternatively, collect 3 times every 15 hrs, but this increases the volume to spin. Keep the supernatant at 4° C. until collection is complete.
k. Filter the medium through a 0.45 um, CA (cellulose acetate) filter and store at 4° C.
l. Spin the supernatant at 19.4 krpm, for 2 hours at room temperature. Use Beckman conical tubes (#358126).
m. Resuspend the pellets in 1 ml total Hank's Balanced Salt Solution (or PBS), for day 1 collection.
n. Pool all pellets from 3 collections and load on top of 1.5 ml of 20% sucrose (in PBS or HBSS).
o. Spin the supernatant 21 krpm for 2 hours at room temperature in a SW55 rotor (Beckman tubes #326819) and resuspend the pellet in 100 ul×2 (200 ul final).
p. Shake on vortex at low speed for a 30 minutes at room temperature.
q. Prepare 20 ul aliquots of the concentrated particles and store at −80° C. until use.
r. Take 1 ul of the concentrated virus and add 89 ul PBS+10 ul of 5% Triton and store at −20° C. for p24 (plasmid particle) ELISA.

IV. A plasmid particle ELISA can be carried out as follows:
a. Set the standards and the samples in 96-well dish. Leave wells A1, B1 and C1 empty. Put 200 ul PBS in wells D1 thru H1, and 270 ul PBS in all the sample wells (4 wells per sample, 2 samples per column).
b. For assay standards, make pre-dilutions of p24 positive control in PBS. Well A1−5 ul positive control+245 ul PBS; well B1−20 ul from well A+780 ul PBS. Put 200 ul of well B suspension in well C1 and in D1. From D1, perform two-fold serial dilutions in the following wells to H1 (200 ul each passage).
c. From 1: 100 dilutions of the supernatants, add 30 ul to the first well containing 270 ul PBS. From this, perform 10-fold dilutions in the following 3 wells (30 ul+270 ul).
d. Wash the assay plate with the required number of strips 3 times in wash buffer.
e. Transfer 195 ul from the dilution plate to the assay plate. Add 200 ul PBS in well B1, leave A1 empty.
f. Cover with adhesive paper and incubate overnight at 40° C. or 2 hr at 37° C.
g. Wash 6 times in wash buffer.
h. Add 100 ul detector antibody (undiluted) to all wells EXCEPT A1. Cover and incubate 1 hr at 37° C.
i. Wash 6 times in wash buffer, add 100 ul streptavidin (diluted 100× from the stock) to all wells EXCEPT A1. Cover and incubate 30 min at 37° C.
j. Dilute 1 tablet of substrate in 11 ml of substrate buffer.
k. Wash 6 times in wash buffer and add 100 ul diluted substrate to all wells INCLUDING A1.
l. Incubate 30 minutes at room temperature in the dark.
m. Add 100 ul stop solution to all wells.
n. Read at 490 nm or 492 nm, with a reference filter at 600 nm.

### Example 11

### Sequence of pTomo Plasmid

The following is the nucleic acid sequence of a pTomo plasmid [SEQ ID NO:1]:

caggtggcacttttcggggaaatgtgcgcggaacccctatttgtttattt
ttctaaatacattcaaatatgtatccgctcatgagacaataaccctgata
aatgcttcaataatattgaaaaaggaagagtatgagtattcaacatttcc
gtgtcgcccttattcccttttttgcggcattttgccttcctgtttttgct
cacccagaaacgctggtgaaagtaaaagatgctgaagatcagttgggtgc
acgagtgggttacatcgaactggatctcaacagcggtaagatccttgaga
gttttcgccccgaagaacgttttccaatgatgagcacttttaaagttctg
ctatgtggcgcggtattatcccgtattgacgccgggcaagagcaactcgg
tcgccgcatacactattctcagaatgacttggttgagtactcaccagtca
cagaaaagcatcttacggatggcatgacagtaagagaattatgcagtgct
gccataaccatgagtgataacactgcggccaacttacttctgacaacgat
cggaggaccgaaggagctaaccgcttttttgcacaacatgggggatcatg
taactcgccttgatcgttgggaaccggagctgaatgaagccataccaaac
gacgagcgtgacaccacgatgcctgtagcaatggcaacaacgttgcgcaa
actattaactggcgaactacttactctagcttcccggcaacaattaatag
actggatggaggcggataaagttgcaggaccacttctgcgctcggccctt
ccggctggctggtttattgctgataaatctggagccggtgagcgtgggtc
tcgcggtatcattgcagcactggggccagatggtaagccctcccgtatcg
tagttatctacacgacggggagtcaggcaactatggatgaacgaaataga
cagatcgctgagataggtgcctcactgattaagcattggtaactgtcaga
ccaagtttactcatatatactttagattgatttaaaacttcatttttaat
ttaaaaggatctaggtgaagatcctttttgataatctcatgaccaaaatc
ccttaacgtgagttttcgttccactgagcgtcagaccccgtagaaaagat
caaaggatcttcttgagatcctttttttctgcgcgtaatctgctgcttgc
aaacaaaaaaaccaccgctaccagcggtggtttgtttgccggatcaagag
ctaccaactctttttccgaaggtaactggcttcagcagagcgcagatacc
aaatactgtccttctagtgtagccgtagttaggccaccacttcaagaact
ctgtagcaccgcctacatacctcgctctgctaatcctgttaccagtggct
gctgccagtggcgataagtcgtgtcttaccgggttggactcaagacgata
gttaccggataaggcgcagcggtcgggctgaacggggggttcgtgcacac
agcccagcttggagcgaacgacctacaccgaactgagatacctacagcgt
gagctatgagaaagcgccacgcttcccgaagggagaaaggcggacaggta
tccggtaagcggcagggtcggaacaggagagcgcacgagggagcttccag
ggggaaacgcctggtatctttatagtcctgtcgggtttcgccacctctga
cttgagcgtcgatttttgtgatgctcgtcaggggggcggagcctatggaa
aaacgccagcaacgcggcctttttacggttcctggccttttgctggcctt
ttgctcacatgttctttcctgcgttatcccctgattctgtggataaccgt
attaccgcctttgagtgagctgataccgctcgccgcagccgaacgaccga
gcgcagcgagtcagtgagcgaggaagcggaagagcgcccaatacgcaaac
cgcctctccccgcgcgttggccgattcattaatgcagctggcacgacagg
tttcccgactggaaagcgggcagtgagcgcaacgcaattaatgtgagtta
gctcactcattaggcaccccaggctttacactttatgcttccggctcgta
tgttgtgtggaattgtgagcggataacaatttcacacaggaaacagctat
gaccatgattacgccaagcgcgcaattaaccctcactaaagggaacaaaa
gctggagctgcaagcttaatgtagtcttatgcaatactcttgtagtcttg
caacatggtaacgatgagttagcaacatgccttacaaggagagaaaaagc
accgtgcatgccgattggtggaagtaaggtggtacgatcgtgccttatta
ggaaggcaacagacgggtctgacatggattggacgaaccactgaattgcc
gcattgcagagatattgtatttaagtgcctagctcgatacaataaacggg
tctctctggttagaccagatctgagcctgggagctctctggctaactagg
gaacccactgcttaagcctcaataaagcttgccttgagtgcttcaagtag
tgtgtgcccgtctgttgtgtgactctggtaactagagatccctcagaccc
ttttagtcagtgtggaaaatctctagcagtggcgcccgaacagggacctg
aaagcgaaagggaaaccagagctctctcgacgcaggactcggcttgctga
agcgcgcacggcaagaggcgaggggcggcgactggtgagtacgccaaaaa
ttttgactagcggaggctagaaggagagagatgggtgcgagagcgtcagt
attaagcgggggagaattagatcgcgatgggaaaaaattcggttaaggcc
agggggaaagaaaaaatataaattaaaacatatagtatgggcaagcaggg
agctagaacgattcgcagttaatcctggcctgttagaaacatcagaaggc
tgtagacaaatactgggacagctacaaccatcccttcagacaggatcaga
agaacttagatcattatataatacagtagcaaccctctattgtgtgcatc
aaaggatagagataaaagacaccaaggaagctttagacaagatagaggaa
gagcaaaacaaaagtaagaccaccgcacagcaagcggccgctgatcttca
gacctggaggaggagatatgagggacaattggagaagtgaattatataaa
tataaagtagtaaaaattgaaccattaggagtagcacccaccaaggcaaa
gagaagagtggtgcagagagaaaaaagagcagtgggaataggagctttgt
tccttgggttcttgggagcagcaggaagcactatgggcgcagcctcaatg
acgctgacggtacaggccagacaattattgtctggtatagtgcagcagca
gaacaatttgctgagggctattgaggcgcaacagcatctgttgcaactca
cagtctggggcatcaagcagctccaggcaagaatcctggctgtggaaaga
tacctaaaggatcaacagctcctggggatttggggttgctctggaaaact
catttgcaccactgctgtgccttggaatgctagttggagtaataaatctc
tggaacagattggaatcacacgacctggatggagtgggacagagaaatta
acaattacacaagcttaatacactccttaattgaagaatcgcaaaaccag
caagaaaagaatgaacaagaattattggaattagataaatgggcaagttt
gtggaattggtttaacataacaaattggctgtggtatataaaattattca
taatgatagtaggaggcttggtaggtttaagaatagtttttgctgtactt
tctatagtgaatagagttaggcagggatattcaccattatcgtttcagac
ccacctcccaaccccgaggggacccgacaggcccgaaggaatagaagaag
aaggtggagagagagacagagacagatccattcgattagtgaacggatct
cgacggttaacttttaaaagaaaaggggggattggggggtacagtgcagg
ggaaagaatagtagacataatagcaacagacatacaaactaaagaattac
aaaaacaaattacaaaaattcaaaattttatcgataagcttgggagttcc
gcgttacataacttacggtaaatggcccgcctggctgaccgcccaacgac
ccccgcccattgacgtcaataatgacgtatgttcccatagtaacgccaat
agggactttccattgacgtcaatgggtggagtatttacggtaaactgccc
acttggcagtacatcaagtgtatcatatgccaagtacgccccctattgac
gtcaatgacggtaaatggcccgcctggcattatgcccagtacatgacctt
atgggactttcctacttggcagtacatctacgtattagtcatcgctatta
ccatggtgatgcggttttggcagtacatcaatgggcgtggatagcggttt
gactcacggggatttccaagtctccaccccattgacgtcaatgggagttt
gttttggcaccaaaatcaacgggactttccaaaatgtcgtaacaactccg
ccccattgacgcaaatgggcggtaggcgtgtacggtgggaggtctatata
agcagagctcgtttagtgaaccgtcagatcgcctggagacgccatccacg
ctgttttgacctccatagaagacaccgactctAGAGCATAACTTCGTATA
ATGTATGCTATACGAAGTTATACCGCCATGGCCTCCTCCGAGGACGTCAT
CAAGGAGTTCATGCGCTTCAAGGTGCGCATGGAGGGCTCCGTGAACGGCC
ACGAGTTCGAGATCGAGGGCGAGGGCGAGGGCCGCCCCTACGAGGGCACC
CAGACCGCCAAGCTGAAGGTGACCAAGGGCGGCCCCCTGCCCTTCGCCTG
GGACATCCTGTCCCCTCAGTTCCAGTACGGCTCCAAGGCCTACGTGAAGC
ACCCCGCCGACATCCCCGACTACTTGAAGCTGTCCTTCCCCGAGGGCTTC
AAGTGGGAGCGCGTGATGAACTTCGAGGACGGCGGCGTGGTGACCGTGAC
CCAGGACTCCTCCCTGCAGGACGGCGAGTTCATCTACAAGGTGAAGCTGC
GCGGCACCAACTTCCCCTCCGACGGCCCCGTAATGCAGAAGAAGACCATG
GGCTGGGAGGCCTCCACCGAGCGGATGTACCCCGAGGACGGCGCCCTGAA
GGGCGAGATCAAGATGAGGCTGAAGCTGAAGGACGGCGGCCACTACGACG
CCGAGGTCAAGACCACCTACATGGCCAAGAAGCCCGTGCAGCTGCCCGGC
GCCTACAAGACCGACATCAAGCTGGACATCACCTCCCACAACGAGGACTA
CACCATCGTGGAACAGTACGAGCGCGCCGAGGGCCGCCACTCCACCGGCG
CCTAAATAACTTCGTATAATGTATGCTATACGAAGTTATGGATCCGCCCC
TCTCCCTCCCCCCCCCCTAACGTTACTGGCCGAAGCCGCTTGGAATAAGG
CCGGTGTGCGTTTGTCTATATGTTATTTTCCACCATATTGCCGTCTTTTG
GCAATGTGAGGGCCCGGAAACCTGGCCCTGTCTTCTTGACGAGCATTCCT
AGGGGTCTTTCCCCTCTCGCCAAAGGAATGCAAGGTCTGTTGAATGTCGT
GAAGGAAGCAGTTCCTCTGGAAGCTTCTTGAAGACAAACAACGTCTGTAG
CGACCCTTTGCAGGCAGCGGAACCCCCCACCTGGCGACAGGTGCCTCTGC
GGCCAAAAGCCACGTGTATAAGATACACCTGCAAAGGCGGCACAACCCCA
GTGCCACGTTGTGAGTTGGATAGTTGTGGAAAGAGTCAAATGGCTCTCCT
CAAGCGTATTCAACAAGGGGCTGAAGGATGCCCAGAAGGTACCCCATTGT
ATGGGATCTGATCTGGGGCCTCGGTGCACATGCTTTACATGTGTTTAGTC
GAGGTTAAAAAAACGTCTAGGCCCCCCGAACCACGGGGACGTGGTTTTCC
TTTGAAAAACACGATGATAATATGGCCACAACCATGGTGAGCAAGGGCGA
GGAGCTGTTCACCGGGGTGGTGCCCATCCTGGTCGAGCTGGACGGCGACG
TAAACGGCCACAAGTTCAGCGTGTCCGGCGAGGGCGAGGGCGATGCCACC
TACGGCAAGCTGACCCTGAAGTTCATCTGCACCACCGGCAAGCTGCCCGT
GCCCTGGCCCACCCTCGTGACCACCCTGACCTACGGCGTGCAGTGCTTCA
GCCGCTACCCCGACCACATGAAGCAGCACGACTTCTTCAAGTCCGCCATG
CCCGAAGGCTACGTCCAGGAGCGCACCATCTTCTTCAAGGACGACGGCAA
CTACAAGACCCGCGCCGAGGTGAAGTTCGAGGGCGACACCCTGGTGAACC
GCATCGAGCTGAAGGGCATCGACTTCAAGGAGGACGGCAACATCCTGGGG
CACAAGCTGGAGTACAACTACAACAGCCACAACGTCTATATCATGGCCGA
CAAGCAGAAGAACGGCATCAAGGTGAACTTCAAGATCCGCCACAACATCG
AGGACGGCAGCGTGCAGCTCGCCGACCACTACGAGCAGAACACCCCCATC
GGCGACGGCCCCGTGCTGCTGCCCGACAACCACTACCTGAGCACCCAGTC
CGCCCTGAGCAAAGACCCCAACGAGAAGCGCGATCACATGGTCCTGCTGG
AGTTCGTGACCGCCGCCGGGATCACTCTCGGCATGGACGAGCTGTACAAG
taaagcggccgcgtcgacaatcaacctctggattacaaaatttgtgaaag
attgactggtattcttaactatgttgctccttttacgctatgtggatacg
ctgctttaatgcctttgtatcatgctattgcttcccgtatggctttcatt
ttctcctccttgtataaatcctggttgctgtctctttatgaggagttgtg
gcccgttgtcaggcaacgtggcgtggtgtgcactgtgtttgctgacgcaa
cccccactggttggggcattgccaccacctgtcagctcctttccgggact
ttcgctttccccctccctattgccacggcggaactcatcgccgcctgcct
tgcccgctgctggacaggggctcggctgttgggcactgacaattccgtgg
tgttgtcggggaagctgacgtcctttccatggctgctcgcctgtgttgcc
acctggattctgcgcgggacgtccttctgctacgtcccttcggccctcaa
tccagcggaccttccttcccgcggcctgctgccggctctgcggcctcttc
cgcgtcttcgccttcgccctcagacgagtcggatctccctttgggccgcc
tccccgcctggaattcgagctcggtacctttaagaccaatgacttacaag
gcagctgtagatcttagccactttttaaaagaaaaggggggactggaagg
gctaattcactcccaacgaagacaagatctgctttttgcttgtactgggt
ctctctggttagaccagatctgagcctgggagctctctggctaactaggg
aacccactgcttaagcctcaataaagcttgccttgagtgcttcaagtagt
gtgtgcccgtctgttgtgtgactctggtaactagagatccctcagaccct
tttagtcagtgtggaaaatctctagcagtagtagttcatgtcatcttatt
attcagtatttataacttgcaaagaaatgaatatcagagagtgagaggaa
cttgtttattgcagcttataatggttacaaataaagcaatagcatcacaa
atttcacaaataaagcauttcactgcattctagttgtggtttgtccaaac
tcatcaatgtatcttatcatgtctggctctagctatcccgcccctaactc
cgcccagttccgcccattctccgccccatggctgactaattttttttatt
tatgcagaggccgaggccgcctcggcctctgagctattccagaagtagtg
aggaggcttttttggaggcctaggcttttgcgtcgagacgtacccaattc
gccctatagtgagtcgtattacgcgcgctcactggccgtcgttttacaac
gtcgtgactgggaaaaccctggcgttacccaacttaatcgccttgcagca
catccccctttcgccagctggcgtaatagcgaagaggcccgcaccgatcg
cccttcccaacagttgcgcagcctgaatggcgaatggcgcgacgcgccct
gtagcggcgcattaagcgcggcgggtgtggtggttacgcgcagcgtgacc
gctacacttgccagcgccctagcgcccgctcctttcgctttcttcccttc
ctttctcgccacgttcgccggctttccccgtcaagctctaaatcgggggc
tccctttagggttccgatttagtgctttacggcacctcgaccccaaaaaa
cttgattagggtgatggttcacgtagtgggccatcgccctgatagacggt
ttttcgccctttgacgttggagtccacgttctttaatagtggactcttgt
tccaaactggaacaacactcaaccctatctcggtctattcttttgattta
taagggattttgccgatttcggcctattggttaaaaaatgagctgattta
acaaaaatttaacgcgaattttaacaaaatattaacgtttacaatttcc

### Example 12

### Summary of all the Injections of Tomo Lentiviral Vectors Expressing Oncogene(s) into GFAP-Cre or GFAP-Cre/p53^{+/-} Mice

| | area of | | tumor |
|---|---|---|---|
| strain | injection | oncogene(s) | incidence |
| GFAP-Cre | CTX | Mock | 0/10 |
| GFAP-Cre | HP | Mock | 0/10 |
| GFAP-Cre | SVZ | Mock | 0/10 |
| GFAP-Cre | CTX | H-Ras | 0/9 |
| GFAP-Cre | HP | H-Ras | 0/10 |
| GFAP-Cre | SVZ | H-Ras | 0/10 |
| GFAP-Cre | CTX | AKT | 0/8 |
| GFAP-Cre | HP | AKT | 0/10 |
| GFAP-Cre | SVZ | AKT | 0/9 |
| GFAP-Cre | CTX | H-Ras + AKT | 0/9 |
| GFAP-Cre | HP | H-Ras + AKT | 5/12 |
| GFAP-Cre | SVZ | H-Ras + AKT | 1/9 |
| GFAP-Cre/p53^{+/-} | CTX | Mock | 0/10 |
| GFAP-Cre/p53^{+/-} | HP | Mock | 0/9 |
| GFAP-Cre/p53^{+/-} | SVZ | Mock | 0/10 |
| GFAP-Cre/p53^{+/-} | CTX | H-Ras | 0/9 |
| GFAP-Cre/p53^{+/-} | HP | H-Ras | 6/10 |
| GFAP-Cre/p53^{+/-} | SVZ | H-Ras | 1/9 |
| GFAP-Cre/p53^{+/-} | CTX | AKT | 0/10 |
| GFAP-Cre/p53^{+/-} | HP | AKT | 0/10 |
| GFAP-Cre/p53^{+/-} | SVZ | AKT | 0/10 |
| GFAP-Cre/p53^{+/-} | CTX | H-Ras + AKT | 1/15 |
| GFAP-Cre/p53^{+/-} | HP | H-Ras + AKT | 17/17 |
| GFAP-Cre/p53^{+/-} | SVZ | H-Ras + AKT | 12/16 |

CTX: cortex,
HP: hippocampus,
SVZ: subventricular zone

### Example 13

### Vector Sequences for Pancreatic Tumor Models

The nucleic acid sequences provided below are used, e.g., with viral particles as described herein, to induce pancreatic tumors by direct injection, e.g., without the Cre/lox system. Schematic illustrations of the vectors are also shown in FIG. 28.

[SEQ ID NO: 2] provides a tissue specific promoter; CAII: carbonic anhydrase II promoter to induce expression in ductal tissue, having the following nucleic acid sequence:

cttagaacaagtaagtctaaacatgaatcatatttgtttggatgaaattg
actgaaataaatagtaaagagtgattttgagatacatattgcaaaaatgt
ccactaataacttatccttgtatttatctgtatattttcaaaggtaagta
atgtgatgtcaaggccgacacaatcacattaaactgtgtcctgcttcccc
aactgtaactctaatactattgccctcagagattgcagagaccacagaac
cgaactccttcccacaaacccccggatttttctgtggcaaaatcccttgt
aggaaatgaattctctctatatctcaaaacagcctcacttacccagtagg
ccctgtaagcacagtgtctcaggtccacaatacttttagatgcccaggaa
aatgtttccttttaaaataagaacaaaaaagaaaaaaattaggtgttggt
taaagaaaatgtttaaatatataatattaagatatttatctttatgtcag
tgttggtataaatgcgtgtttatgaaaaagggacccaccaaggtgaaatg
ccgtaggacccacaaatgtcataatgctggctggcctcaaaatgtccttc
ctattaaactattttgaatgattcctaggtatgccagatgccaatttttc
ttggccagccagggaacgtctcatgtctttggaagaaattatctttctct
tactggattcttctgagaaaagtgatttgttaaaagttttggttatttca
aatcaatatgtactcaactaggacgtctcctcacccacgttaatttttta
aagaggaggaaagtaggagaagatgagcgcttcatgtaaggctctgccag
cctacgaggaaatgcaatcctccaggctcgcccgagcgtgggctgcgggc
aggggactgtggggccgttgtctcgttttattccacggccgtgcagcaga
gggcgcgtgcctctcagcactcatgctgcgcgctcccctgcgcctctagg
atcgtatccctatttgccccaaggtcatagctcctaacccagcttgccag
ccccaggccggggacaccagagactgagcccttcgcgctggagaccccgg
cgcgggtggggcgggagggctccggggccaagagacaggtcacaatgggg
acaggggacgaaaggcgccgggggtccgggtcccgagcagtccccgccgc
cgccagactccgcagccgggagggggatggggtgcgcgtagaggctccgc
ggcccgggttggacggaggagcccaggagccaccgcagccgccgcacgcc
cagagctccgagcttcgctgccagcccaggacaccggggccctgccgtgg
gccgaggggagccgggcggcgggagaggagccccggagccccggagcccc
ggagccccgccgggcgccagccgagagggcgttttccccgcccccggaga
ctcgcccgcccggcccgccccgccccgagcgcggggagttcacctccgcc
cgtcaccacctccccttgtcgcctaggtccacccgagccccctcccccgg
gccgcccccgagcacgaagttggcgggagcctataaaagctggtgccggc
gcgacccgcggacacacagtgcaGGCGCCCAAGCCGCCGCCGCCAGATCG
GTGCCGATTCCT

[SEQ ID NO:3] provides the nucleic acid sequences for tissue specific promoter; Ela1: elastase 1 for use, e.g., in acinar cells, as follows:

gtacactgtagctgacttcagatgcaccagaagagggcatcagatcccat
tacagatggttgtgagccaccatgtggttgctgggaattgaactcaggac
ctctggaagagcaagcagtcagtgctcttaacctctgagccatctctcca
gtcccattttgaattttttattattgtgtttacctatctatgtgtttgtg
gtgtgtccatgtgtgcatgagggcatgcgtgtggcaagcaggggacaact
cgcaggaatccatccactcttccaccatgtgagttgcgaggattgaacgt
aggtggtcagcttggtggcaaacggcttcaccctctcagccatctcgaaa
gcccgctaaggatttgcaggctgcgtctcaactgaggtcaactcagttga
cttgggttaagtggatgccgtccttgttctgtctttgaatatcagataaa
tgagttgacttaaaattttgttcatttgtactttcatgtcacctgcgctt
ttccctgccttctacccatggccctgcccagctggcaggaggaaggtcag
caaagctgctgataagaggcgtataaagagggctcggttcatggcaaggg
GACAGTGGTCTACTCTCTCC

[SEQ ID NO: 4] provides a nucleic acid coding sequence of interest for a pancreatic oncogene; K-Ras G12D mutation.

ATGACTGAGTATAAACTTGTGGTGGTTGGAGCTGATGGCGTAGGCAAGAG
CGCCTTGACGATACAGCTAATTCAGAATCACTTTGTGGATGAGTACGACC
CTACGATAGAGGACTCCTACAGGAAACAAGTAGTAATTGATGGAGAAACC
TGTCTCTTGGATATTCTCGACACAGCAGGTCAAGAGGAGTACAGTGCAAT
GAGGGACCAGTACATGAGAACTGGGGAGGGCTTTCTTTGTGTATTTGCCA
TAAATAATACTAAATCATTTGAAGATATTCACCATTATAGAGAACAAATT
AAAAGAGTAAAGGACTCTGAAGATGTGCCTATGGTCCTGGTAGGGAATAA
GTGTGATTTGCCTTCTAGAACAGTAGACACGAAACAGGCTCAGGAGTTAG
CAAGGAGTTACGGGATTCCGTTCATTGAGACCTCAGCAAAGACAAGACAG
GGTGTTGACGATGCCTTCTATACATTAGTCCGAGAAATTCGAAAACATAA
AGAAAAGATGAGCAAAGATGGGAAGAAGAAGAAGAAGAAGTCAAGGACAA
GGTGTACAGTTATGTGA

[SEQ ID NO:5] provides a sequence for inactivating the following tumor suppressors; IVS-miRNA with s1-p53, si-p16 and si-smad4.

CTAGAGAATTAATTCGCTGTCTGCGAGGGCCAGCTGTTGGGGTGAGTACT
CCCTCTCAAAAGCGGGCATGACTTCTGCGCTAAGATTGTCAGTTTCCAAA
AACGAGGAGGATTTGATATTCACCTGGCCCGCGGTAGTGAGTCGACCAGT
GGATCCTGGAGGCTTGCTGAAGGCTGTATGCTGGGAGCTATTACACATGT
ACTTGTTTTGGCCACTGACTGACAAGTACATGTAATAGCTCCCAGGACAC
AAGGCCTGTTACTAGCACTCACATGGAACAAATGGCCCAGATCTGGCCGC
ACTCGACCAGTGGATCCTGGAGGCTTGCTGAAGGCTGTATGCTGgggcgt
gcttgagctgaagctatgcccgtGTTTTGGCCACTGACTGACacgggcat
agcttgctcaagcacgcccCAGGACACAAGGCCTGTTACTAGCACTCACA
TGGAACAAATGGCCCAGATCTGGCCGCACTCGACCAGTGGATCCTGGAGG
CTTGCTGAAGGCTGTATGCTGTATGATGGTAAGTAGCTGGGTTTTGGCCA
CTGACTGACCCAGCTACACCATCATACAGGACACAAGGCCTGTTACTAGC
ACTCACATGGAACAAATGGCCCAGATCTGGCCGCACTCGAGATATCTAGA
CCCAGCTTTCTTGTACAAAGCTTGAGGTGTGGCAGGCTTGAGATCTGGCC
ATACACTTGAGTGACAATGACATCCACTTTGCCTTTCTCTCCACAGGTGT
CCACTCCCAGGTCCAACTGCAGGTCGGGATCCAAA

### Example 14

### Generating Lentiviral Vectors for Injection

The methods provided herein are optionally used to transduce a small number of pancreatic exocrine cells with lentiviral vectors capable of generating activated Kras, shRNAi capable of knocking down TP53, Ink4/p16 and Smad4 to develop into invasive ductal tumors that metastsize into various organs and show chromosomal instability with a time course that mimics human pancreatic cancer.

A): siRNA Sequences for the Knockdown.

Several sequences specific to mouse (but not human) p53, Ink4/p16 and Smad4 were selected, and tested separately with an H1 promoter to make sure these sequences efficiently knockdown endogenous mouse proteins. For safety reasons, the Kras-G12D mutant was cloned into a tet-on vector. FIG. 24 shows that the expression of Kras is tightly controlled by doxycycline. It has been previously shown by Hingorani et al. (101) that overexpression of mutated Kras and mutated p53 induces tumors within 6 months. Vectors generating Kras & knock down of p53 in the pancreas have been injected as well as vectors that constitutively express IkBαM (dominant negative) or IKK2 (dominant active) to test the role of NFkB pathway in pancreatic cancer development. FIG. 25 illustrates a test of the combination of these vectors with the Kras/s1-p53 vector.

B): Lentiviral Vector Transduction of Pancreas:

Two routes of transduction were tested: parenchyma vs. common bile duct injection (CBD). CBD injection: Since the bile duct is even thinner than the 30G insulin needle, the dye was injected through the gall bladder (and ligated after the injection). The total volume could be >100 ul, but the dye went directly to the duodenum (percentage of the dye that retrograded to the pancreas is unknown). Direct parenchyma injection: several sites were tried for injection in the pancreas parenchyma; the head of the pancreas (circled region in FIG. 26) is the preferred site for the injection. If a tumor develops here, it will easily block the main pancreatic duct and give severe phenotype. It's also the most common site for most human pancreatic cancers to develop. FIG. 27 shows an efficient infection after injection of 2 ul (titer of 10E9) CAG-GFP virus directly to the parenchyma.

C) An Alternative Approach to Induce Pancreatic Cancer.

As many papers indicated that endogenous expression level of mutated Kras is critical to induce the PanIn and PDAC, another approach to generate tumors is presented herein. A mouse strain (LSL-KrasG12D) was obtained, with Loxp-Kras G12D cassette knocked in the endogenous allele. A single lentiviral vector with tissue specific cre and microRNA with 3 shRNAs to p53, Ink4/p16, and Smad4 is optionally used to activate the Kras mutation and knockdown the tumor suppressors in a cell-type specific manner (FIG. 27). See also FIG. 28 presenting a lentiviral vector without the lox recombination sites that is optionally used.

### Example 15

### Further Studies of Pancreatic Tumor Models

A) Generation of a lentiviral vector capable of inducing pancreatic carcinoma. A single lentiviral vector expressing a Kras^{G12D} transgene as well as three siRNAs against the Ink4/p16, p53 and SMAD4 tumor suppressors with a microRNA backbone will be expressed under a variety of promoters capable of expression in ductal or centroacinar cells. For example, an elastase promoter is optionally used for expression in acinar cells; a carbonic anhydrase II promoter for expression in ductal cells, and a nestin promoter for expression on pancreatic progenitor cells.

With Kras knock in mice available, lenti-Cre viral vectors under the control of promoters expressing genes in acinar/ductal cells can be generated. These promoters include 5.0 kB fragment of Keratin-5 gene (26), 1.6 kB human carbonic anhydrase 11 (27), and approximately 0.5 kB rat elastase promoter-enhancer sequences (28). A few days after infection, animals are sacrificed and expression of GFP in the pancreas is determined. We will inject the promoter specific lenti-Cre GFP in both Kras and Kras/p53+/− or Kras+si p53 to test if tumors are observed.

B) Induction of pancreatic ductal cancer. Once the expression pattern of the above vector has been ascertained, then the viral vectors containing the transgene and siRNAs can be constructed and recombinant viruses injected, e.g., in the parenchyma of the pancreas. Tumors arising are analyzed histologically. Clonality of the tumors arising can be analyzed to assess whether additional mutations are necessary for tumorigenesis. Because the same cells can be repeatedly transduced with lentiviral vectors, the order and combinations of various genetic alterations can be examined and altered.

C) Role of inflammation. Because NFκB is activated in a variety of pancreatic cancers and cell lines, it has been assumed that it may be involved in inflammatory responses which promote inflammation (29). Furthermore in many pancreatic cancers, high levels of TNF alpha, a direct target of NFκB and probable cause of cachexia can be observed. Appropriate pancreatic cells can be transduced as described herein with a lentiviral vector generating constitutively active IkB Kinase 2 (IKK2) to induce NFκB activity or a lentiviral vector generating IkBM (transdominant inhibitor of NFκB) to block NFκB activity (30). These models are optionally used to determine whether an inflammatory response accentuates pancreatic cancers.

### LITERATURE CITED

1. Lin R C, Matesic D F, Marvin M, McKay R D and Brustle O (1995) Re-expression of the intermediate filament nestin in reactive astrocytes. Neurobiol Dis, 2: 79-85.
2. Houchens D P, Ovejera A A, Riblet S M and Slagel D E (1983) Human brain tumor xenografts in nude mice as a chemotherapy model. Eur J Cancer Clin Oncol, 19: 799-805.
3. Hu B, Guo P, Fang Q, Tao H Q, Wang D, Nagane M, Huang H J, Gunji Y, Nishikawa R, Alitalo K, Cavenee W K and Cheng S Y (2003) Angiopoietin-2 induces human glioma invasion through the activation of matrix metalloprotease-2. Proc Natl Acad Sci USA, 100: 8904-8909.
4. Bao S, Wu Q, Sathornsumetee S, Hao Y, Li Z, Hjelmeland A B, Shi Q, McLendon R E, Bigner D D and Rich J N (2006) Stem Cell-like Glioma Cells Promote Tumor Angiogenesis through Vascular Endothelial Growth Factor. Cancer Res, 66: 7843-7848.
5. Garkavtsev I, Kozin S V, Chemova O, Xu L, Winkler F, Brown E, Barnett G H and Jain R K (2004) The candidate tumour suppressor protein ING4 regulates brain tumour growth and angiogenesis. Nature, 428: 328-332.
6. Kleihues P, Louis D N, Scheithauer B W, Rorke L B, Reifenberger G, Burger P C and Cavenee W K (2002) The WHO classification of tumors of the nervous system. J Neuropathol Exp Neurol, 61: 215-225; discussion 226-219.
7. Maher E A, Furnari F B, Bachoo R M, Rowitch D H, Louis D N, Cavenee W K and DePinho R A (2001) Malignant glioma: genetics and biology of a grave matter. Genes Dev, 15: 1311-1333.
8. Guha A, Dashner K, Black P M, Wagner J A and Stiles C D (1995) Expression of PDGF and PDGF receptors in human astrocytoma operation specimens supports the existence of an autocrine loop. Int J Cancer, 60: 168-173.
9. Trojan J, Blossey B K, Johnson T R, Rudin S D, Tykocinski M, Ilan J and Ilan J (1992) Loss of tumorigenicity of rat glioblastoma directed by episome-based antisense cDNA transcription of insulin-like growth factor I. Proc Natl Acad Sci USA, 89: 4874-4878.
10. Wong A J, Bigner S H, Bigner D D, Kinzler K W, Hamilton S R and Vogelstein B (1987) Increased expression of the epidermal growth factor receptor gene in malignant gliomas is invariably associated with gene amplification. Proc Natl Acad Sci USA, 84: 6899-6903.
11. Ekstrand A J, Longo N, Hamid M L, Olson J J, Liu L, Collins V P and James C D (1994) Functional characterization of an EGF receptor with a truncated extracellular domain expressed in glioblastomas with EGFR gene amplification. Oncogene, 9: 2313-2320.
12. Davis R J (2000) Signal transduction by the JNK group of MAP kinases. Cell, 103: 239-252.
13. Li J, Yen C, Liaw D, Podsypanina K, Bose S, Wang S I, Puc J, Miliaresis C, Rodgers L, McCombie R, Bigner S H, Giovanella B C, Ittmann M, Tycko B, Hibshoosh H, Wigler M H and Parsons R (1997) PTEN, a putative protein tyrosine phosphatase gene mutated in human brain, breast, and prostate cancer. Science, 275: 1943-1947.
14. Steck P A, Pershouse M A, Jasser S A, Yung W K, Lin H, Ligon A H, Langford L A, Baumgard M L, Hattier T, Davis T, Frye C, Hu R, Swedlund B, Teng D H and Tavtigian S V (1997) Identification of a candidate tumour suppressor gene, MMAC1, at chromosome 10q23.3 that is mutated in multiple advanced cancers. Nat Genet, 15: 356-362.
15. Lee J O, Yang H, Georgescu M M, Di Cristofano A, Maehama T, Shi Y, Dixon J E, Pandolfi P and Pavletich N P (1999) Crystal structure of the PTEN tumor suppressor: implications for its phosphoinositide phosphatase activity and membrane association. Cell, 99: 323-334.
16. Stambolic V, Suzuki A, de la Pompa J L, Brothers G M, Mirtsos C, Sasaki T, Ruland J, Penninger J M, Siderovski D P and Mak T W (1998) Negative regulation of PKB/Akt-dependent cell survival by the tumor suppressor PTEN. Cell, 95: 29-39.
17. Costello J F, Berger M S, Huang H S and Cavenee W K (1996) Silencing of p16/CDKN2 expression in human gliomas by methylation and chromatin condensation. Cancer Res, 56: 2405-2410.
18. Kamijo T, Bodner S, van de Kamp E, Randle D H and Sherr C J (1999) Tumor spectrum in ARF-deficient mice. Cancer Res, 59: 2217-2222.
19. Serrano M, Hannon G J and Beach D (1993) A new regulatory motif in cell-cycle control causing specific inhibition of cyclin D/CDK4. Nature, 366: 704-707.
20. Zhang Y, Xiong Y and Yarbrough W G (1998) ARF promotes MDM2 degradation and stabilizes p53: ARF-INK4a locus deletion impairs both the Rb and p53 tumor suppression pathways. Cell, 92: 725-734.
21. Levine A J (1997) p53, the cellular gatekeeper for growth and division. Cell, 88: 323-331.
22. Louis D N (1994) The p53 gene and protein in human brain tumors. J Neuropathol Exp Neurol, 53: 11-21.
23. Reifenberger G, Liu L, Ichimura K, Schmidt E E and Collins V P (1993) Amplification and overexpression of the MDM2 gene in a subset of human malignant gliomas without p53 mutations. Cancer Res, 53: 2736-2739.
24. Marumoto T, Zhang D and Saya H (2005) Aurora-A—a guardian of poles. Nat Rev Cancer, 5: 42-50.
25. Andrews P D (2005) Aurora kinases: shining lights on the therapeutic horizon? Oncogene, 24: 5005-5015.
26. Carmena M and Earnshaw W C (2003) The cellular geography of aurora kinases. Nat Rev Mol Cell Biol, 4: 842-854.
27. Brown J R, Koretke K K, Birkeland M L, Sanseau P and Patrick DR (2004) Evolutionary relationships of Aurora kinases: implications for model organism studies and the development of anti-cancer drugs. BMC Evol Biol, 4: 39.
28. Meraldi P, Honda R and Nigg E A (2004) Aurora kinases link chromosome segregation and cell division to cancer susceptibility. Curr Opin Genet Dev, 14: 29-36.
29. Earnshaw W C and Cooke C A (1991) Analysis of the distribution of the INCENPs throughout mitosis reveals the existence of a pathway of structural changes in the chromosomes during metaphase and early events in cleavage furrow formation. J Cell Sci, 98 (Pt 4): 443-461.
30. Li X, Sakashita G, Matsuzaki H, Sugimoto K, Kimura K, Hanaoka F, Taniguchi H, Furukawa K and Urano T (2004) Direct association with inner centromere protein (INCENP) activates the novel chromosomal passenger protein, Aurora-C. J Biol Chem, 279: 47201-47211.
31. Sasai K, Katayama H, Stenoien D L, Fujii S, Honda R, Kimura M, Okano Y, Tatsuka M, Suzuki F, Nigg E A, Eamshaw W C, Brinkley W R and Sen S (2004) Aurora-C kinase is a novel chromosomal passenger protein that can complement Aurora-B kinase function in mitotic cells. Cell Motil Cytoskeleton, 59: 249-263.
32. Meraldi P, Honda R and Nigg E A (2002) Aurora-A overexpression reveals tetraploidization as a major route to centrosome amplification in p53−/− cells. Embo J, 21: 483-492.
33. Bischoff J R, Anderson L, Zhu Y, Mossie K, Ng L, Souza B, Schryver B, Flanagan P, Clairvoyant F, Ginther C, Chan C S, Novotny M, Slamon D J and Plowman G D (1998) A homologue of Drosophila aurora kinase is oncogenic and amplified in human colorectal cancers. Embo J, 17: 3052-3065.
34. Li Q and Verma I M (2002) NF-kappaB regulation in the immune system. Nat Rev Immunol, 2: 725-734.
35. Verma I M, Stevenson J K, Schwarz E M, Van Antwerp D and Miyamoto S (1995) Rel/NF-kappa B/I kappa B family: intimate tales of association and dissociation. Genes Dev, 9: 2723-2735.
36. Karin M and Ben-Neriah Y (2000) Phosphorylation meets ubiquitination: the control of NF-[kappa]B activity. Annu Rev Immunol, 18: 621-663.
37. Li Z W, Chu W, Hu Y, Delhase M, Deerinck T, Ellisman M, Johnson R and Karin M (1999) The IKKbeta subunit of IkappaB kinase (IKK) is essential for nuclear factor kappaB activation and prevention of apoptosis. J Exp Med, 189: 1839-1845.
38. Senftleben U, Cao Y, Xiao G, Greten F R, Krahn G, Bonizzi G, Chen Y, Hu Y, Fong A, Sun S C and Karin M (2001) Activation by IKKalpha of a second, evolutionary conserved, NF-kappa B signaling pathway. Science, 293: 1495-1499.
39. Huang S, Robinson J B, Deguzman A, Bucana C D and Fidler I J (2000) Blockade of nuclear factor-kappaB signaling inhibits angiogenesis and tumorigenicity of human ovarian cancer cells by suppressing expression of vascular endothelial growth factor and interleukin 8. Cancer Res, 60: 5334-5339.
40. Bond M, Fabunmi R P, Baker A H and Newby A C (1998) Synergistic upregulation of metalloproteinase-9 by growth factors and inflammatory cytokines: an absolute requirement for transcription factor NF-kappa B. FEBS Lett, 435: 29-34.
41. Hesselager G and Holland E C (2003) Using mice to decipher the molecular genetics of brain tumors. Neurosurgery, 53: 685-694; discussion 695.
42. Kobayashi N, Allen N, Clendenon N R and Ko L W (1980) An improved rat brain-tumor model. J Neurosurg, 53: 808-815.
43. Finkelstein S D, Black P, Nowak T P, Hand C M, Christensen S and Finch P W (1994) Histological characteristics and expression of acidic and basic fibroblast growth factor genes in intracerebral xenogeneic transplants of human glioma cells. Neurosurgery, 34: 136-143.
44. Holland E C, Celestino J, Dai C, Schaefer L, Sawaya R E and Fuller G N (2000) Combined activation of Ras and Akt in neural progenitors induces glioblastoma formation in mice. Nat Genet, 25: 55-57.
45. Sausville E A (2004) Aurora kinases dawn as cancer drug targets. Nat Med, 10: 234-235.
46. Harrington E A, Bebbington D, Moore J, Rasmussen R K, Ajose-Adeogun A O, Nakayama T, Graham J A, Demur C, Hercend T, Diu-Hercend A, Su M, Golec J M and Miller K M (2004) VX-680, a potent and selective small-molecule inhibitor of the Aurora kinases, suppresses tumor growth in vivo. Nat Med, 10: 262-267.
47. Marumoto T, Honda S, Hara T, Nitta M, Hirota T, Kohmura E and Saya H (2003) Aurora-A kinase maintains the fidelity of early and late mitotic events in HeLa cells. J Biol Chem, 278: 51786-51795.
48. Vescovi A L, Galli R and Reynolds B A (2006) Brain tumour stem cells. Nat Rev Cancer, 6: 425-436.
49. Holland E C (2001) Gliomagenesis: genetic alterations and mouse models. Nat Rev Genet, 2: 120-129.
50. Kafri T, Blomer U, Peterson D A, Gage F H and Verma I M (1997) Sustained expression of genes delivered directly into liver and muscle by lentiviral vectors. Nat Genet, 17: 314-317.
51. Abremski K, Hoess R and Sternberg N (1983) Studies on the properties of P1 site-specific recombination: evidence for topologically unlinked products following recombination. Cell, 32: 1301-1311.
52. Guha A, Feldkamp M M, Lau N, Boss G and Pawson A (1997) Proliferation of human malignant astrocytomas is dependent on Ras activation. Oncogene, 15: 2755-2765.
53. Ding H, Roncari L, Shannon P, Wu X, Lau N, Karaskova J, Gutmann D H, Squire J A, Nagy A and Guha A (2001) Astrocyte-specific expression of activated p21-ras results in malignant astrocytoma formation in a transgenic mouse model of human gliomas. Cancer Res, 61: 3826-3836.
54. Iyer V, Pumiglia K and DiPersio C M (2005) Alpha3beta1 integrin regulates MMP-9 mRNA stability in immortalized keratinocytes: a novel mechanism of integrin-mediated MMP gene expression. J Cell Sci, 118: 1185-1195.
55. Reynolds B A and Weiss S (1992) Generation of neurons and astrocytes from isolated cells of the adult mammalian central nervous system. Science, 255: 1707-1710.
56. Hopewell J W (1975) The subependymal plate and the genesis of gliomas. J Pathol, 117: 101-103.
57. Lantos P L and Cox D J (1976) The origin of experimental brain tumours: a sequential study. Experientia, 32: 1467-1468.
58. Vick N A, Lin M J and Bigner D D (1977) The role of the subependymal plate in glial tumorigenesis. Acta Neuropathol (Berl), 40: 63-71.
59. Malatesta P, Hack M A, Hartfuss E, Kettenmann H, Klinkert W, Kirchhoff F and Gotz M (2003) Neuronal or glial progeny: regional differences in radial glia fate. Neuron, 37: 751-764.
60. Wang S I, Puc J, Li J, Bruce J N, Cairns P, Sidransky D and Parsons R (1997) Somatic mutations of PTEN in glioblastoma multiforme. Cancer Res, 57: 4183-4186.
61. Veselska R, Kuglik P, Cejpek P, Svachova H, Neradil J, Loja T and Relichova J (2006) Nestin expression in the cell lines derived from glioblastoma multiforme. BMC Cancer, 6: 32.
62. Lang F F, Miller D C, Koslow M and Newcomb E W (1994) Pathways leading to glioblastoma multiforme: a molecular analysis of genetic alterations in 65 astrocytic tumors. J Neurosurg, 81: 427-436.
63. Watanabe K, Tachibana O, Sata K, Yonekawa Y, Kleihues P and Ohgaki H (1996) Overexpression of the EGF receptor and p53 mutations are mutually exclusive in the evolution of primary and secondary glioblastomas. Brain Pathol, 6: 217-223; discussion 223-214.
64. Sidransky D, Mikkelsen T, Schwechheimer K, Rosenblum M L, Cavanee W and Vogelstein B (1992) Clonal expansion of p53 mutant cells is associated with brain tumour progression. Nature, 355: 846-847.
65. Marr R A, Rockenstein B, Mukherjee A, Kindy M S, Hersh L B, Gage F H, Verma I M and Masliah E (2003) Neprilysin gene transfer reduces human amyloid pathology in transgenic mice. J Neurosci, 23: 1992-1996.
66. Marr R A, Guan H, Rockenstein E, Kindy M, Gage F H, Verma I, Masliah E and Hersh L B (2004) Neprilysin regulates amyloid Beta peptide levels. J Mol Neurosci, 22: 5-11.
67. Singer O, Marr R A, Rockenstein E, Crews L, Coufal N G, Gage F H, Verma I M and Masliah E (2005) Targeting BACE1 with siRNAs ameliorates Alzheimer disease neuropathology in a transgenic model. Nat Neurosci, 8: 1343-1349.
68. Hovatta I, Tennant R S, Helton R, Marr R A, Singer O, Redwine J M, Ellison J A, Schadt EE, Verma I M, Lockhart D J and Barlow C (2005) Glyoxalase 1 and glutathione reductase 1 regulate anxiety in mice. Nature, 438: 662-666.
69. Keles G E, Lamborn K R and Berger M S (2001) Low-grade hemispheric gliomas in adults: a critical review of extent of resection as a factor influencing outcome. J Neurosurg, 95: 735-745.
70. Li Q, Lu Q, Estepa G and Verma I M (2005) Identification of 14-3-3sigma mutation causing cutaneous abnormality in repeated-epilation mutant mouse. Proc Natl Acad Sci USA, 102: 15977-15982.
71. Schadt E E, Li C, Ellis B and Wong W H (2001) Feature extraction and normalization algorithms for high-density oligonucleotide gene expression array data. J Cell Biochem Suppl, Suppl 37: 120-125.
72. Irizarry R A, Hobbs B, Collin F, Beazer-Barclay Y D, Antonellis K J, Scherf U and Speed TP (2003) Exploration, normalization, and summaries of high density oligonucleotide array probe level data. Biostatistics, 4: 249-264.
73. Fuller G N, Hess K R, Rhee C H, Yung W K, Sawaya R A, Bruner J M and Zhang W (2002) Molecular classification of human diffuse gliomas by multidimensional scaling analysis of gene expression profiles parallels morphology-based classification, correlates with survival, and reveals clinically-relevant novel glioma subsets. Brain Pathol, 12: 108-116.
74. Phillips H S, Kharbanda S, Chen R, Forrest W F, Soriano R H, Wu T D, Misra A, Nigro J M, Colman H, Soroceanu L, Williams P M, Modrusan Z, Feuerstein B G and Aldape K (2006) Molecular subclasses of high-grade glioma predict prognosis, delineate a pattern of disease progression, and resemble stages in neurogenesis. Cancer Cell, 9: 157-173.
75. Tiscornia G, Tergaonkar V, Galimi F and Verma I M (2004) CRE recombinase-inducible RNA interference mediated by lentiviral vectors. Proc Natl Acad Sci USA, 101: 7347-7351.
76. Tergaonkar V, Bottero V, Ikawa M, Li Q and Verma I M (2003) IkappaB kinase-independent IkappaBalpha degradation pathway: functional NF-kappaB activity and implications for cancer therapy. Mol Cell Biol, 23: 8070-8083.
77. Kunitoku N, Sasayama T, Marumoto T, Zhang D, Honda S, Kobayashi O, Hatakeyama K, Ushio Y, Saya H and Hirota T (2003) CENP-A phosphorylation by Aurora-A in prophase is required for enrichment of Aurora-B at inner centromeres and for kinetochore function. Dev Cell, 5: 853-864.
78. Amoh Y, Yang M, Li L, Reynoso J, Bouvet M, Moossa A R, Katsuoka K and Hoffman R M (2005) Nestin-linked green fluorescent protein transgenic nude mouse for imaging human tumor angiogenesis. Cancer Res, 65: 5352-5357.
79. Hoffman R M (2005) The multiple uses of fluorescent proteins to visualize cancer in vivo. Nat Rev Cancer, 5: 796-806.
80. Emanuel S, Rugg C A, Gruninger R H, Lin R, Fuentes-Pesquera A, Connolly P J, Wetter SK, Hollister B, Kruger W W, Napier C, Jolliffe L and Middleton S A (2005) The in vitro and in vivo effects of JNJ-7706621: a dual inhibitor of cyclin-dependent kinases and aurora kinases. Cancer Res, 65: 9038-9046.
81. Schopf L, Savinainen A, Anderson K, Kujawa J, DuPont M, Silva M, Siebert E, Chandra S, Morgan J, Gangurde P, Wen D, Lane J, Xu Y, Hepperle M, Harriman G, Ocain T and Jaffee B (2006) IKKbeta inhibition protects against bone and cartilage destruction in a rat model of rheumatoid arthritis. Arthritis Rheum, 54: 3163-3173.
82. Shih A H and Holland E C (2004) Developmental neurobiology and the origin of brain tumors. J Neurooncol, 70: 125-136.
83. Pfeifer A, Brandon E P, Kootstra N, Gage F H and Verma I M (2001) Delivery of the Cre recombinase by a self-deleting lentiviral vector: efficient gene targeting in vivo. Proc Natl Acad Sci USA, 98: 11450-11455.
84. Jemal A, Murray T, Samuels A, Ghafoor A, Ward E, Thun M J. 2003. CA Cancer J. Clin. 2003; 53:5-26.
85. Li D, Xie K, Wolff R, Abbruzzese J L. Lancet. 2004; 363:1049-1057.
86. Jaffee E M, Hruban R H, Canto M, Kern S E. Cancer Cell. 2:25-28.
87. Hruban R H, Wilentz R E, Kern S E. Am J. Pathol. 156:1821-1825.
88. Klein W M, Hruban R H, Klein-Szanto A J, Wilentz R E. Mod Pathol. 15:441-447.
89. DiGiuseppe J A, Hruban R H, Goodman S N, Polak M, van den Berg F M, Allison D C, Cameron J L, Offerhaus G J. Am. J Clin Pathol. 101:684-688.
90. Wilentz R E, Geradts J, Maynard R, Offerhaus G J, Kang M, Goggins M, Yeo C J, Kern S E, Hruban R H. Cancer Res. 58:4740-4744.
91. Luttges J, Schlehe B, Menke M A, Vogel I, Henne-Bruns D, Kloppel G. Cancer. 85:1703-1710.
92. Hahn S A, Greenhalf B, Ellis I, Sina-Frey M, Rieder H, Korte B, Gerdes B, Kress R, Ziegler A, Raeburn J A, Campra D, Grutzmann R, Rehder H, Rothmund M, Schmiegel W, Neoptolemos J P, Bartsch D K. J Natl Cancer Inst. 95:214-221.
93. Yamamoto H, Itoh F, Nakamura H, Fukushima H, Sasaki S, Perucho M, Imai K. Cancer Res. 61:3139-3144.
94. Hingorani SR, Petricoin E F, Maitra A, Rajapakse V, King C, Jacobetz M A, Ross S, Conrads T P, Veenstra T D, Hitt B A, Kawaguchi Y, Johann D, Liotta L A, Crawford H C, Putt M E, Jacks T, Wright C V, Hruban R H, Lowy A M, Tuveson D A. Cancer Cell. 4:437-450.
95. Brembeck F H, Schreiber F S, Deramaudt T B, Craig L, Rhoades B, Swain G, Grippo P, Stoffers D A, Silberg D G, Rustgi A K. Cancer Res. 63:2005-2009.
96. Serrano M, Lee H, Chin L, Cordon-Cardo C, Beach D, DePinho R A. Cell. 85:27-37.
97. Kamijo T, Weber J D, Zambetti G, Zindy F, Roussel M F, Sherr C J. Proc Natl Acad Sci USA. 95:8292-8297.
98. Krimpenfort P, Quon K C, Mooi W J, Loonstra A, Berns A. Nature. 413:83-86.
99. Sharpless N E, Bardeesy N, Lee K H, Carrasco D, Castrillon D H, Aguirre A J, Wu EA, Horner J W, DePinho R A. Nature. 413:86-91.
100. Aguirre A J, Bardeesy N, Sinha M, Lopez L, Tuveson D A, Horner J, Redston M S, DePinho R A. Genes Dev. 17:3112-3126.
101. Hingorani S R, Wang L, Multani A S, Combs C, Deramaudt T B, Hruban R H, Rustgi A K, Chang S, Tuveson D A. Cancer Cell. 7:469-483.
102. Siveke J T, Schmid R M. Cancer Cell. 7:405-407.
103. Van Dyke T, Jacks T. Cell. 108:135-144.
104. Hruban R H, Adsay N V, Albores-Saavedra J, Anver M R, Biankin A V, Boivin G P, Furth E E, Furukawa T, Klein A, Klimstra D S, Kloppel G, Lauwers G Y, Longnecker D S, Luttges J, Maitra A, Offerhaus G J, Perez-Gallego L, Redston M, Tuveson D A. Cancer Res. 66:95-106.
105. Bissell M J, Labarge M A. Cancer Cell. 7:17-23.
106. Verma I M, Weitzman W D. Annu Rev Biochem. 2005; 74:711-738.
107. Tiscornia G, Singer O, Ikawa M, Verma I M. Proc. Natl. Acad. Sci. (USA). 2003; 100:1844-1848.
108. Tiscornia G, Tergaonkar V, Galimi F, Verma I M. Proc Natl Acad. Sci. 2004; 101:7347-7351.
109. Muller-Decker K, Furstenberger G, Annan N, Kucher D, Pohl-Arnold A, Steinbauer B, Esposito I, Chiblak S, Friess H, Schirmacher P, Berger I. Gastroenterology. 130:2165-2178.
110. Inada A, Nienaber C, Fonseca S, Bonner-Weir S. Dev Dyn. 235:1571-1577.
111. Lewis B C, Klimstra D S, Varmus H E. Genes Dev. 17:3127-3138.
112. Garcea G, Dennison A R, Steward WP, Berry D P. Pancreatology. 2005; 5:514-529.
113. Tergaonkar V, Li Q, Verma I M. In: Liou H-C, ed. NFkB/Rell Transcription Factor Family: Landes/Eurekah; 2006.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, many of the techniques and apparatus described above can be used in various combinations.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of generating localized foci of transduced cells in vivo, the method comprising:
providing a vector system comprising one or more recombinant nucleic acid comprising an encoded sequence of interest;
providing an animal;
injecting the vector system into an organ of the animal at a desired location; and,
transducing the recombinant nucleic acid from the vector system into target cells at the desired location;
thereby generating a localized foci of transduced target cells in the organ.

2. The method of claim 1, wherein the transduced cells are selected from the group consisting of: an astrocyte from an adult animal, a brain cell, neuronal cell, a pancreatic cell, a prostate cell, a breast cell, a liver cell, a skin cell, a differentiated cell, and a lung cell.

3. The method of claim 1, wherein the encoded sequence of interest is selected from the group consisting of: Ras, myc, src, AKT, NFκB, Aurora-A kinase, BAFF, Kras, a sequence coding for an agent that represses expression or activity of a tumor suppressor, a sequence encoding a serine kinase, a sequence encoding a G-protein, a sequence encoding a threonine kinase and a sequence encoding a tyrosine kinase.

4. The method of claim 1, wherein the vector system comprises: a packaging cell that comprises the recombinant nucleic acid, a lentiviral packaging plasmid that encodes a lentiviral gag protein and a pseudotyping plasmid that encodes an envelope protein that is heterologous to the lentiviral gag protein.

5. The method of claim 1, wherein injecting comprises: inserting the tip of a needle to the desired location and depositing the vector from the needle tip.

6. The method of claim 1, wherein injecting comprises injection of 10⁸ IU or less of the lentiviral vector and transducing 100 or fewer cells at the location.

7. The method of claim 1, further comprising generating foci of transduced cells at the desired location and not in other organs or tissues of the animal.

8. Treating a disease state in the animal by transducing cells of the animal according to the method of claim 1.

9. The method of claim 1, wherein the vector system is a lentiviral vector system.

10. A composition comprising a recombinant nucleic acid that comprises:
a sequence comprising a first lox recombination site and a second lox recombination site bracketing a nucleic acid sequence encoding a stuffer sequence;
a promoter sequence upstream from the lox recombination sites;
a coding sequence of interest and a first marker nucleic acid sequence encoding a first marker, each downstream from the lox recombination sites; and,
a lentiviral packaging site (psi).

11. The composition of claim 10, wherein the recombinant nucleic acid is contained within a packaging cell that also comprises a lentiviral packaging plasmid that encodes a lentiviral gag protein and a pseudotyping plasmid that encodes an envelope protein that is heterologous to the lentiviral gag protein.

12. The composition of claim 10, further comprising a pseudotyped lentiviral vector, which vector packages the recombinant nucleic acid in a lentiviral capsid.

13. The composition of claim 10, further comprising a target cell.

14. The composition of claim 13 wherein the target cell expresses Cre recombinase.

15. The composition of claim 10, wherein the stuffer sequence encodes a second marker or a stop codon.

16. The composition of claim 10, wherein the promoter is an inducible promoter.

17. The composition of claim 10, wherein the sequence of interest encodes an oncogenic polypeptide or an anti-tumor suppressor moiety.

18. A non-human recombinant laboratory animal, comprising:
a recombinant Cre recombinase, expressed in one or more cell or tissue of the animal; and,
a recombinant nucleic acid that encodes a sequence comprising:
i) a first lox recombination site and a second lox recombination site bracketing a nucleic acid sequence encoding a stuffer sequence, or comprising a recombinant nucleic acid that encodes a sequence comprising recombined lox recombination sites;
ii) a promoter sequence upstream from the recombined lox recombination sites or upstream from the first and second lox sites;
iii) a coding sequence of interest and a first marker nucleic acid sequence encoding a first marker, each downstream from the recombined lox recombination sites or downstream from the first and second lox sites; and,
iv) a lentiviral packaging site (psi).

19. The animal of claim 18, wherein the animal is selected from the group consisting of: a mouse, a rat, and a monkey.

20. The animal of claim 18, wherein the coding sequence of interest is selected from the group consisting of: ras, myc, src, AKT, Aurora-A kinase, BAFF, Kras, a sequence coding for an agent that represses expression or activity of a tumor suppressor, a sequence encoding a serine kinase, a sequence encoding a G-protein, a sequence encoding a threonine kinase, and a sequence encoding a tyrosine kinase.

21. A method of screening for modulators of tumorigenesis, the method comprising:
providing a non-human recombinant laboratory animal as in claim 18;
contacting the animal with a putative modulator of tumorigenesis; and,
detecting one or more changes in one or more tumor-associated parameters in the animal.

22. The method of claim 21, wherein the coding sequence of interest is selected from the group consisting of: Ras, myc, src, AKT, Aurora-A kinase, NFκB, BAFF, Kras, a sequence coding for an agent that represses expression or activity of a tumor suppressor, a sequence encoding a serine kinase, a sequence encoding a G-protein, a sequence encoding a threonine kinase, and a sequence encoding a tyrosine kinase.

23. The method of claim 21, wherein the putative modulator is selected from the group consisting of: an anti-cancer drug, VX680, hesperidin, ZM447-439, and a molecule from a small molecule library.

24. The method of claim 21, wherein the tumor-associated parameter is selected from the group consisting of: increased cellular density, necrosis, invasion of surrounding tissue by the cells, nuclear pleomorphism, giant cell formation, increased mitotic cells, lack of contact inhibition, and increased nucleus to cytoplasm ratio.


SEQUENCE LISTING (5 sequences)

SEQ ID NO: 1 - DNA, 8753 bp, Artificial Sequence
  SEQ ID NO 1 is the nucleic acid sequence of a pTomo plasmid
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggttaacttttaaaagaaaaggggggattggggggtacagtgcaggggaaagaatagtagacataatagcaacagacatacaaactaaagaattacaaaaacaaattacaaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagagcataacttcgtataatgtatgctatacgaagttataccgccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaaataacttcgtataatgtatgctatacgaagttatggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 2 - DNA, 1662 bp, Mus musculus
cttagaacaagtaagtctaaacatgaatcatatttgtttggatgaaattgactgaaataaatagtaaagagtgattttgagatacatattgcaaaaatgtccactaataacttatccttgtatttatctgtatattttcaaaggtaagtaatgtgatgtcaaggccgacacaatcacattaaactgtgtcctgcttccccaactgtaactctaatactattgccctcagagattgcagagaccacagaaccgaactccttcccacaaacccccggatttttctgtggcaaaatcccttgtaggaaatgaattctctctatatctcaaaacagcctcacttacccagtaggccctgtaagcacagtgtctcaggtccacaatacttttagatgcccaggaaaatgtttccttttaaaataagaacaaaaaagaaaaaaattaggtgttggttaaagaaaatgtttaaatatataatattaagatatttatctttatgtcagtgttggtataaatgcgtgtttatgaaaaagggacccaccaaggtgaaatgccgtaggacccacaaatgtcataatgctggctggcctcaaaatgtccttcctattaaactattttgaatgattcctaggtatgccagatgccaatttttcttggccagccagggaacgtctcatgtctttggaagaaattatctttctcttactggattcttctgagaaaagtgatttgttaaaagttttggttatttcaaatcaatatgtactcaactaggacgtctcctcacccacgttaattttttaaagaggaggaaagtaggagaagatgagcgcttcatgtaaggctctgccagcctacgaggaaatgcaatcctccaggctcgcccgagcgtgggctgcgggcaggggactgtggggccgttgtctcgttttattccacggccgtgcagcagagggcgcgtgcctctcagcactcatgctgcgcgctcccctgcgcctctaggatcgtatccctatttgccccaaggtcatagctcctaacccagcttgccagccccaggccggggacaccagagactgagcccttcgcgctggagaccccggcgcgggtggggcgggagggctccggggccaagagacaggtcacaatggggacaggggacgaaaggcgccgggggtccgggtcccgagcagtccccgccgccgccagactccgcagccgggagggggatggggtgcgcgtagaggctccgcggcccgggttggacggaggagcccaggagccaccgcagccgccgcacgcccagagctccgagcttcgctgccagcccaggacaccggggccctgccgtgggccgaggggagccgggcggcgggagaggagccccggagccccggagccccggagccccgccgggcgccagccgagagggcgttttccccgcccccggagactcgcccgcccggcccgccccgccccgagcgcggggagttcacctccgcccgtcaccacctccccttgtcgcctaggtccacccgagccccctcccccgggccgcccccgagcacgaagttggcgggagcctataaaagctggtgccggcgcgacccgcggacacacagtgcaggcgcccaagccgccgccgccagatcggtgccgattcct

SEQ ID NO: 3 - DNA, 620 bp, Mus musculus
gtacactgtagctgacttcagatgcaccagaagagggcatcagatcccattacagatggttgtgagccaccatgtggttgctgggaattgaactcaggacctctggaagagcaagcagtcagtgctcttaacctctgagccatctctccagtcccattttgaattttttattattgtgtttacctatctatgtgtttgtggtgtgtccatgtgtgcatgagggcatgcgtgtggcaagcaggggacaactcgcaggaatccatccactcttccaccatgtgagttgcgaggattgaacgtaggtggtcagcttggtggcaaacggcttcaccctctcagccatctcgaaagcccgctaaggatttgcaggctgcgtctcaactgaggtcaactcagttgacttgggttaagtggatgccgtccttgttctgtctttgaatatcagataaatgagttgacttaaaattttgttcatttgtactttcatgtcacctgcgcttttccctgccttctacccatggccctgcccagctggcaggaggaaggtcagcaaagctgctgataagaggcgtataaagagggctcggttcatggcaaggggacagtggtctactctctcc

SEQ ID NO: 4 - DNA, 567 bp, Homo sapiens
atgactgagtataaacttgtggtggttggagctgatggcgtaggcaagagcgccttgacgatacagctaattcagaatcactttgtggatgagtacgaccctacgatagaggactcctacaggaaacaagtagtaattgatggagaaacctgtctcttggatattctcgacacagcaggtcaagaggagtacagtgcaatgagggaccagtacatgagaactggggagggctttctttgtgtatttgccataaataatactaaatcatttgaagatattcaccattatagagaacaaattaaaagagtaaaggactctgaagatgtgcctatggtcctggtagggaataagtgtgatttgccttctagaacagtagacacgaaacaggctcaggagttagcaaggagttacgggattccgttcattgagacctcagcaaagacaagacagggtgttgacgatgccttctatacattagtccgagaaattcgaaaacataaagaaaagatgagcaaagatgggaagaagaagaagaagaagtcaaggacaaggtgtacagttatgtga

SEQ ID NO: 5 - DNA, 785 bp, Artificial Sequence
  SEQ ID NO 5 provides a sequence for inactivating the following tumor suppressors;  IVS-miRNA with si-p53, si-p16 and si-smad4
ctagagaattaattcgctgtctgcgagggccagctgttggggtgagtactccctctcaaaagcgggcatgacttctgcgctaagattgtcagtttccaaaaacgaggaggatttgatattcacctggcccgcggtagtgagtcgaccagtggatcctggaggcttgctgaaggctgtatgctgggagctattacacatgtacttgttttggccactgactgacaagtacatgtaatagctcccaggacacaaggcctgttactagcactcacatggaacaaatggcccagatctggccgcactcgaccagtggatcctggaggcttgctgaaggctgtatgctggggcgtgcttgagctgaagctatgcccgtgttttggccactgactgacacgggcatagcttgctcaagcacgccccaggacacaaggcctgttactagcactcacatggaacaaatggcccagatctggccgcactcgaccagtggatcctggaggcttgctgaaggctgtatgctgtatgatggtaagtagctgggttttggccactgactgacccagctacaccatcatacaggacacaaggcctgttactagcactcacatggaacaaatggcccagatctggccgcactcgagatatctagacccagctttcttgtacaaagcttgaggtgtggcaggcttgagatctggccatacacttgagtgacaatgacatccactttgcctttctctccacaggtgtccactcccaggtccaactgcaggtcgggatccaaa